(19) United States
(12) Patent Application Publication
(10) Pub. No.: US 2023/0248855 A1
(43) Pub. Date: Aug. 10, 2023

(54) HER3 RADIOIMMUNOTHERAPY FOR THE TREATMENT OF SOLID CANCERS

(71) Applicant: ACTINIUM PHARMACEUTICALS, INC., NEW YORK, NY (US)

(72) Inventors: DALE L LUDWIG, Rockaway, NJ (US); Eileen Geoghegan, Mamaroneck, NY (US); Sandesh Seth, New York, NY (US); Paul Diamond, Fort Lee, NJ (US)

(21) Appl. No.: 18/146,149

(22) Filed: Dec. 23, 2022

Related U.S. Application Data
(63) Continuation-in-part of application No. 17/532,919, filed on Nov. 22, 2021.
Application No. 17/532,919 is a continuation-in-part of application No. PCT/US2021/056259, filed on Oct. 22, 2021.
(60) Provisional application No. 63/118,181, filed on Nov. 25, 2020.
(60) Provisional application No. 63/116,225, filed on Nov. 20, 2020.
(60) Provisional application No. 63/250,725, filed on Sep. 30, 2021.
(60) Provisional application No. 63/226,699, filed on Jul. 28, 2021.
(60) Provisional application No. 63/104,386, filed on Oct. 22, 2020.

(51) Int. Cl. A61K 51/10; A61K 45/06; A61P 35/00

(52) U.S. Cl. CPC A61K 51/1051; A61K 45/06; A61P 35/00

(57) ABSTRACT

Provided are compositions and methods for treating a solid cancer such as a HER3-positive tumor in a subject by administering an effective amount of a HER3-targeting agent labeled with a radionuclide such as ²²⁵Ac, ¹⁷⁷Lu, ¹³¹I, ⁹⁰Y, ²¹³Bi, ²¹¹At, ²¹³Bi, ²²⁷Th, or ²¹²Pb, alone or in combination with other therapeutic agents or modalities such as VEGF or VEGFR inhibitors. The effective amount of the radiolabeled HER3-targeting agent may be a maximum tolerated dose administered in a single bolus or in fractionated doses that together equal the maximum tolerated dose.

### CROSS-REFERENCE TO RELATED APPLICATIONS

This application is a continuation-in-part of U.S. application Ser. No. 17/532,919 filed Nov. 22, 2021, which

is a continuation-in-part of International Application No. PCT/US21/56259 filed Oct. 22, 2021, which claims priority to each of U.S. provisional application Ser. No. 63/250,725 filed Sep. 30, 2021, 63/226,699 filed Jul. 28, 2021, and 63/104,386 filed Oct. 22, 2020;

claims the benefit of U.S. provisional patent application No. 63/118,181 filed Nov. 25, 2020; and

claims the benefit of U.S. provisional patent application No. 63/116,225 filed Nov. 20, 2020,

each of the foregoing applications hereby incorporated by reference in its entirety.

### SEQUENCE LISTING

The instant application contains a Sequence Listing which has been submitted electronically in XML format and is hereby incorporated by reference in its entirety. Said ASCII copy, created on Dec. 18, 2022, is named ATNM-010PCT_SL_ST26.xml and is 164,722 bytes in size.

### FIELD OF THE INVENTION

The present invention relates to the field of radiotherapeutics.

### BACKGROUND OF THE INVENTION

ErbB3/HER3 is a type I transmembrane glycoprotein that is a member of the erythroblastic oncogene B (ErbB) family of tyrosine kinase receptors (EGFR, HER2, HER3, and HER4). Signaling through HER3 can be activated in a ligand-dependent or ligand-independent manner. In the absence of ligand, HER3 receptor molecules are normally expressed at the cell surface as monomers with a conformation that prevents receptor dimerization. In this conformation, the dimerization loop of subdomain II makes intramolecular contact with a pocket on subdomain IV. Binding of a HER3 ligand such as a neuregulin (NRG), e.g. NRG1 (also known as heregulin, HRG) or NRG2, to subdomains I and III of the extracellular region causes a conformational change that results in exposure of the dimerization loop of subdomain II, facilitating receptor dimerization and signaling. Certain cancer-associated mutations in HER3 disrupt this interaction between subdomains II and IV, i.e., the interaction required for formation of the inactive ‘closed’ conformation, and thereby cause constitutive presentation of the dimerization loop and activation of HER3-mediated signaling in the absence of ligand binding.

Antibodies that target HER3 may be employed to target specific cancer cells, particularly certain solid cancers. HER3 is overexpressed in several types of cancers such as breast, gastrointestinal, and pancreatic cancers. A correlation between the expression of HER2/HER3 and the progression from a non-invasive stage to an invasive stage of these cancers has been shown. Agents that interfere with HER3-mediated signaling, such as anti-HER3 antibodies, may enable the establishment of a robust immune response to the cancer cells that would be otherwise inadequate using conventional therapy.

Accordingly, one object of the presently disclosed invention is to provide therapeutically effective radiolabeled HER3 targeting agents, such as for the treatment of HER3-positive cancers. A related object of the presently disclosed invention is to provide therapeutic methods including administration of such a radiolabeled HER3 targeting agent, either alone or in combination with one or more additional therapeutic agents.

### SUMMARY OF THE INVENTION

The present invention provides a HER3 targeting agent, such as a monoclonal antibody, peptide, or small molecule that targets HER3, labeled with a radioisotope, and methods of diagnosing and/or treating HER3-positive (HER3-expressing) cancers using the radiolabeled HER3 targeting agent.

According to certain aspects of the present invention, the radiolabeled HER3 targeting agent useful for diagnostics purposes may be an anti-HER3 antibody, peptide, or small molecule including a radioisotope, such as ¹¹¹In, ⁶⁸Ga, ⁶⁴Cu, ⁸⁹Zr, or ¹⁷⁷Lu.

According to certain other aspects, the radiolabeled HER3 targeting agent useful for therapeutic interventions may be an anti-HER3 antibody, peptide, or small molecule including a radioisotope, such as: ⁶⁴Cu, ⁶⁷Cu, ¹⁴⁹Tb, ²⁰¹Tl, ⁴⁷Sc, ¹³¹I, ¹²⁵I, ¹²³I, ⁹⁰Y, ¹⁷⁷Lu, ¹⁸⁶Re, ¹⁸⁸Re, ⁸⁹Sr, ¹⁵³Sm, ³²P, ²²⁵Ac, ²¹³Bi, ²¹³Po, ²¹¹At, ²¹²Bi, ²¹³Bi, ²²³Ra, ²²⁷Th, ¹⁴⁹Tb, ¹³⁷Cs, ²¹²Pb, or combinations thereof. According to certain preferred aspects, the radiolabeled HER3 targeting agent may include ¹³¹I, ⁹⁰Y, ¹⁷⁷Lu, ²²⁵Ac, ²¹³Bi, ²¹¹At, ²²⁷Th, or ²¹²Pb.

According to certain aspects, the HER3-positive cancer may be a solid tumor.

Therapeutic methods of the presently disclosed invention generally include administering to a patient a therapeutically effective amount of the radiolabeled HER3 targeting agent. According to certain aspects, the effective amount of the radiolabeled HER3 targeting agent may be a maximum tolerated dose (MTD) or may be a fractioned dose wherein the total amount of radiation administered in the fractioned doses is the MTD.

According to certain aspects, provided and/or used is a composition or quantity of the HER3 targeting agent that includes a radiolabeled fraction and a non-radiolabeled fraction of the HER3 targeting agent. As such, an effective amount of the HER3 targeting agent may include a total protein dose of less than 100 mg, such as from 5 mg to 60 mg, or 5 mg to 45 mg. According to certain aspects, the total protein dose may be from 0.001 mg/kg to 3 mg/kg body weight of the subject, such as from 0.005 mg/kg to 2 mg/kg body weight of the subject. According to certain aspects, the total protein dose may be less than 2 mg/kg, or less than 1 mg/kg, less than 0.5 mg/kg, or even less than 0.1 mg/kg. A portion of the total protein dose is radiolabeled (i.e., radio-conjugate) as indicated, wherein the effective amount of the radiolabeled HER3 targeting agent may depend on the specific radioisotope selected. Preferred radioisotopes for therapeutic interventions include ²²⁵Ac, ¹⁷⁷Lu, ¹³¹I, ⁹⁰Y, ²¹³Bi, ²¹¹At, ²²⁷Th, or ²¹²Pb. Thus, the HER3 targeting agent may include a radiolabeled fraction and an unlabeled fraction.

According to certain aspects, an effective amount of a HER3 targeting agent in terms of radiation dose, i.e., of the radiolabeled portion thereof, such as an ²²⁵Ac-anti-HER3 antibody, peptide, or small molecule, may include a dose of 0.1 to 20 μCi/kg body weight of the subject, such as 0.1 to 10 μCi/kg or 0.1 to 5 uCi/kg body weight of the subject, or 0.5 to 20 μCi/kg or 1 to 10 uCi/kg body weight of the subject.

According to certain aspects, the effective amount of the HER3 targeting agent, i.e., the radiolabeled portion thereof, such as an ²²⁵Ac-anti-HER3 antibody, peptide, or small molecule may depend on the configuration of the targeting agent, i.e., full length antibody or antigen-binding antibody fragment (e.g., Fab, Fab₂, minibody, nanobody, etc) such as any of those disclosed herein. For example, when the HER3 targeting agent includes an ²²⁵Ac-anti-HER3 antibody that is a full-length antibody, the dose may be at or below 5 uCi/kg body weight of the subject, such as 0.1 to 5 uCi/kg body weight of the subject. Alternatively, when the HER3 targeting agent includes an ²²⁵Ac-anti-HER3 antibody that is a fragment, the dose may be greater than 5 uCi/kg body weight of the subject, such as 5 to 20 uCi/kg body weight of the subject.

According to certain aspects, the HER3 targeting agent is an anti-HER3 antibody, such as a monoclonal antibody or an antigen binding fragment thereof, such as an IgG or an antigen binding fragment thereof, such as one that binds to an epitope of HER3 recognized by Patritumab from Daiichi Sankyo, Seribantumab (MM-121) from Merrimack Pharmaceuticals, Lumretuzumab from Roche, Elgemtumab from Novartis, GSK2849330 from GlaxoSmithKline, CDX-3379 of Celldex Therapeutics, EV20 and MP-RM-1 from MediPharma, ISU104 from Isu Abxis Co., HMBD-001 (10D1F) from Hummingbird Bioscience Pte., REGN1400 from Regeneron Pharmaceuticals, and/or AV-203 from AVEO Oncology (a/k/a Aveo Pharmaceuticals, Inc., Boston, Mass., USA). According to certain aspects, the anti-HER3 antibody is selected from one or more of Patritumab, Seribantumab, Lumretuzumab, Elgemtumab, AV-203, CDX-3379, or GSK2849330.

According to certain aspects, the HER3 targeting agent may be administered according to a dosing schedule selected from the group consisting of one dose every 7, 10, 12, 14, 20, 24, 28, 35, and 42 days throughout a treatment period, wherein the treatment period includes at least two doses.

According to certain aspects, the HER3 targeting agent may be administered according to a dose schedule that includes 2 doses, such as on days 1 and 5, 6, 7, 8, 9, or 10 of a treatment period, or days 1 and 8 of a treatment period.

According to certain aspects, the HER3 targeting agent may be administered as a single bolus or infusion in a single subject specific dose.

According to certain aspects, the methods may further include administration of one or more further therapeutic agents, such as a chemotherapeutic agent, a small molecule drug, an anti-inflammatory agent, an immunosuppressive agent, an immunomodulatory agent, an antimyeloma agent, a cytokine, or any combination thereof. Exemplary chemotherapeutic agents include at least radiosensitizers that may synergize with the radiolabeled HER3 targeting agent, such as temozolomide, cisplatin, and/or fluorouracil.

According to certain aspects, the methods may further include administration of one or more immune checkpoint therapies. Exemplary immune checkpoint therapies include an antibody against CTLA-4, PD-1, TIM-3, VISTA, BTLA, LAG-3, TIGIT, CD28, OX40, GITR, CD137, CD40, CD40L, CD27, HVEM, PD-L1, PD-L2, PD-L3, PD-L4, CD80, CD86, CD137-L, GITR-L, CD226, B7-H3, B7-H4, BTLA, TIGIT, GALS, KIR, 2B4, CD160, CGEN-15049, or any combination thereof. According to certain aspects, the immune checkpoint therapy may include an antibody against an immune checkpoint protein selected from the group consisting of an antibody against PD-1, PD-L1, PD-L2, CTLA-4, CD137, and a combination thereof.

According to certain aspects, the immune checkpoint therapy may be administered to a subject in an effective amount, such as a dose of 0.1 mg/kg to 50 mg/kg of the patient's body weight, such as 0.1-5 mg/kg, or 5-30 mg/kg.

According to certain aspects, the methods may further include administration of one or more DNA damage response inhibitors (DDRi). Exemplary DDRi agents one or more antibodies or small molecules targeting poly(ADP-ribose) polymerase (i.e., a poly(ADP-ribose) polymerase inhibitor or PARPi). The PARPi may, for example, include olaparib, niraparib, rucaparib, talazoparib, or any combination thereof. According to certain aspects, the PARPi may be provided in a subject effective amount including 0.1 mg/day-1200 mg/day, such as 0.100 mg/day-600 mg/day, or 0.25 mg/day-1 mg/day. Exemplary subject effective amounts include 0.1 mg, 0.25 mg, 0.5 mg, 0.75 mg, 1.0 mg, 100 mg, 200 mg, 300 mg, 400 mg, 500 mg, 600 mg, 700 mg, 750 mg, 800 mg, 900 mg, and 1000 mg, taken orally in one or two doses per day.

Another exemplary DDRi includes an inhibitor of Ataxia telangiectasia mutated (ATM), Ataxia talangiectasia mutated and Rad-3 related (ATR), or Wee1. Exemplary inhibitors of ATM include KU-55933, KU-59403, wortmannin, CP466722, and KU-60019. Exemplary inhibitors of ATR include at least Schisandrin B, NU6027, NVP-BEA235, VE-821, VE-822, AZ20, and AZD6738. Exemplary inhibitors of Wee1 include AZD-1775 (i.e., adavosertib).

According to certain aspects, the methods may further include administration of one or more CD47 blockades. The CD47 blockade may include a monoclonal antibody, SIRPα-Fc fusion protein or other molecule that prevents CD47 binding to SIRPα or otherwise blocks or downregulates the immunosuppressive activity of CD47, such as magrolimab, lemzoparlimab, AO-176, AK117, IMC-002, IBI-188, IBI-322, BI 766063, ZL-1201, AXL148, RRx-001, ES004, SRF231, SHR-1603, TJC4, TTI-621, or TTI-622. Exemplary effective doses for the CD47 blockade include 0.05 to 5 mg/kg patient weight. The CD47 blockade may also include agents that modulate the expression of CD47 and/or SIRPα, such as a nucleic acid approach, e.g., anti-sense, RNAi, or μRNA approaches. Exemplary CD47 blockades also include phosphorodiamidate morpholino oligomers (PMO) that block translation of CD47 such as MBT-001.

According to certain aspects, the methods may further include administration of a combination of further therapeutic agents. Exemplary combinations include at least one or more DDRi and/or one or more immune checkpoint therapies and/or one or more CD47 blockades and/or one or more chemotherapeutics and/or one or more small molecule anti-cancer drugs and/or one or more targeting agents directed against different cancer-associated antigens.

According to certain aspects, the radiolabeled HER3 targeting agent and the one or more further therapeutic agents may be administered simultaneously or sequentially. When more than one additional therapeutic agent is administered, the agents may be administered simultaneously or sequentially.

According to certain aspects of the present invention, the radiolabeled HER3 targeting agent may be a multi-specific targeting agent such as a multi-specific antibody or bispecific antibody, in which at least one part recognizes HER3. Thus, the methods may include administering to the subject an effective amount of a multi-specific antibody, wherein the multi-specific antibody includes: a first target recognition component which specifically binds to an epitope of HER3, and a second target recognition component which specifically binds to a different epitope of HER3 than the first target recognition component, or to an epitope of a different antigen such as a different cancer-associated antigen. According to certain aspects, the HER3 targeting agent is a multi-specific antibody against a first epitope of HER3 and at least a second epitope of HER3, or against HER3 and at least a second (different) antigen. Exemplary multi-specific antibodies that may be radiolabeled for diagnostic and/or therapeutic use according to the invention include bispecific antibodies against HER3/IER2 such as MM-111 from Merrimack Pharmaceuticals or MCLA-128 from Merus N.V.; or against IGF-1R/IER3 such as MM-141 (i.e., Istiratumab) from Merrimack Pharmaceuticals; or against EGFR/IER3 such as MEHD7945A (i.e., Duligotumab) from Roche or any of the cetuximab-based bispecific or multi-specific zybodies from Zyngenia Inc.

According to certain aspects, a composition is provided that includes a mixture of a HER3 targeting agent such as an antibody against HER3 and one or more further targeting agents, such as antibodies, targeting/against one or more different cancer associated antigens, wherein one or more of the HER3 targeting agent and the other targeting agent(s) may be radiolabeled or non-radiolabeled in any combination. An exemplary antibody composition including an antibody mixture includes at least Sym013 from Symphogen having six monoclonal antibodies against EGFR (HER1), HER2, and HER3. In one aspect, one or more of the antibodies of the Sym013 may be radiolabeled in any combination, such as at least a HER3 antibody and none or one or more of the antibodies against EGFR and HER2.

Additional features, advantages, and aspects of the invention may be set forth or apparent from consideration of the following detailed description, drawings if any, and claims. Moreover, it is to be understood that both the foregoing summary of the invention and the following detailed description are exemplary and intended to provide further explanation without limiting the scope of the invention as claimed.

### BRIEF DESCRIPTION OF THE DRAWINGS

FIG. 1 provides the amino acid sequences of the N-terminal region, complementarity determining regions, and variable regions of the heavy and light chains of a HER3 monoclonal antibody that may be embodied in various aspects of the present invention.

FIG. 2 provides the amino acid sequences of the full-length heavy and light chains, with and without leader sequences, of a HER3 monoclonal antibody that may be embodied in various aspects of the present invention.

FIG. 3 shows ELISA assay binding characteristics of an Ac225 labeled DOTA-conjugated anti-HER3 monoclonal antibody versus the unmodified anti-HER3 antibody and a non-specific antibody (IgG), demonstrating that the modifications do not materially affect immune reactivity to HER3.

FIG. 4 is a graph showing the results of flow cytometry assays examining the binding of the 225Ac-HER3-ARC, the unmodified anti-HER3 mAb, non-specific antibody control (IgG), and secondary antibody only control to HER3-positive NCI-H1975 cells (human lung adenocarcinoma, NSCLC) and BxPC-3 cells (human pancreatic adenocarcinoma).

FIG. 5 is a graph showing the in vitro cytotoxic effect of 225Ac-HER3-ARC to HER3-positive cell line NCI-H1975 as a function of radiation dose.

FIG. 6A is a graph showing that 225Ac-HER3-ARC upregulates cell surface calreticulin (CRT) in NCI-H1975 cells.

FIG. 6B is a graph showing that 225Ac-HER3-ARC upregulates CD47 on NCI-H1975 cells.

FIG. 7A is a graph showing results of a phagocytosis assay demonstrating that the combination of 225Ac-HER3-ARC and an anti-CD47 blocking antibody enhanced phagocytosis of BxPC-3 cells versus either treatment alone.

FIG. 7B is a graph showing results of a phagocytosis assay demonstrating that the combination of 225Ac-HER3-ARC and an anti-CD47 blocking antibody enhanced phagocytosis of NCI-H1975 cells versus either treatment alone.

FIG. 8 is graph showing the effects on tumor growth, in a human tumor (NCI-H1975 cell) mouse xenograft model, of a 225Ac-HER3-ARC at different radiation doses and in combination with an anti-CD47 blocking antibody.

FIG. 9 is a graph showing body weight over time for the subjects of the experiment described in FIG. 8.

FIG. 10 is a graph showing the probability of survival over time for the experimental group subjects of the experiment described in FIG. 8

FIG. 11 is a graph showing the comparative effects on tumor growth of vehicle (control), CD47 blocking antibody magrolimab alone, 225Ac-trastuzumab alone, and the combination of magrolimab and 225Ac-trastuzumab in an NGS mouse xenograft model using the HER2-positive SK-OV3 human ovarian cancer cell line.

FIG. 12 is a graph showing the comparative effects on tumor growth of vehicle (control), magrolimab alone, 177Lu-trastuzumab alone, and the combination of magrolimab and 177Lu-trastuzumab in an NGS mouse xenograft model using the SK-OV3 human ovarian cancer cell line.

### DETAILED DESCRIPTION OF THE INVENTION

In one aspect, the presently disclosed invention provides compositions and methods for the treatment of cancers expressing HER3, i.e., HER3-positive cancers. This aspect generally includes administering to a mammalian subject in need of treatment, such as a human patient, an effective amount of a radiolabeled HER3 targeting agent, such as a radiolabeled antibody, peptide, or small molecule targeted to HER3, alone or in combination with one or more additional therapeutic agents and/or therapeutic modalities/treatments.

Additional therapeutic agents and modalities that may be used include, for example, at least one or more immune checkpoint therapy and/or one or more inhibitors of a component of the DNA damage response pathway (i.e., a DNA damage response inhibitor, DDRi, such as one or more agents against poly(ADP-ribose) polymerase, i.e., PARPi) and/or one or more CD47/SIRPα axis blockades and/or one or more chemotherapeutic agents such as radiosensitizers, and/or one or more small molecule oncology drugs such as tyrosine kinase inhibitors, and/or one or more targeting agents against different antigens.

The presently disclosed invention further provides methods for identifying, imaging and/or diagnosing HER3-positive cancers in a subject. The presently disclosed invention further provides methods for identifying, imaging and/or diagnosing HER3-positive cancer in a subject, followed by treating those subjects according to any of the methods disclosed herein.

### Definitions and Abbreviations

The singular forms “a,” “an,” “the” and the like include plural referents unless the context clearly dictates otherwise. Thus, for example, reference to “an” antibody includes both a single antibody and a plurality of different antibodies.

The words “comprising” and forms of the word “comprising” as well as the word “including” and forms of the word “including,” as used in this description and in the claims, do not limit the inclusion of elements beyond what is referred to. Additionally, although throughout the present disclosure various aspects or elements thereof are described in terms of “including” or “comprising,” corresponding aspects or elements thereof described in terms of “consisting essentially of” or “consisting of” are similarly disclosed. For example, while certain aspects of the invention have been described in terms of a method “including” or “comprising” administering a radiolabeled targeting agent, corresponding methods instead reciting “consisting essentially of” or “consisting of” administering the radiolabeled target are also within the scope of said aspects and disclosed by this disclosure.

The term “about” when used in this disclosure in connection with a numerical designation or value, e.g., in describing temperature, time, amount, and concentration, including in the description of a range, indicates a variance of ±10% and, within that larger variance, variances of ±5% or ±1% of the numerical designation or value.

As used herein, “administer”, with respect to a targeting agent such as an antibody, antibody fragment, Fab fragment, or aptamer, means to deliver the agent to a subject's body via any known method suitable for antibody delivery. Specific modes of administration include, without limitation, intravenous, transdermal, subcutaneous, intraperitoneal, intrathecal and intra-tumoral administration. Exemplary administration methods for antibodies may be as substantially described in International Publication No. WO 2016/187514, incorporated by reference herein.

In addition, in this invention, antibodies may, for example, be formulated using one or more routinely used pharmaceutically acceptable carriers and excipients. Such carriers and excipients are well known to those skilled in the art. For example, injectable drug delivery systems include solutions, suspensions, gels, microspheres and polymeric injectables, and can include excipients such as solubility-altering agents (e.g., ethanol, propylene glycol and sucrose) and polymers (e.g., polycaprylactones and PLGA's).

As used herein, the term “antibody” includes, without limitation, (a) an immunoglobulin molecule including two heavy chains and two light chains and which recognizes an antigen; (b) polyclonal and monoclonal immunoglobulin molecules; (c) monovalent and divalent fragments thereof, such as Fab, di-Fab, scFvs, diabodies, minibodies, and nanobodies (sdAb); (d) naturally occurring and non-naturally occurring, such as wholly synthetic antibodies, IgG-Fc-silent, and chimeric; and (e) bi-specific and multi-specific forms thereof. Immunoglobulin molecules may derive from any of the commonly known classes, including but not limited to IgA, secretory IgA, IgG and IgM. IgG subclasses are also well known to those in the art and include, but are not limited to, human IgG1, IgG2, IgG3 and IgG4. The N-terminus of each chain defines a “variable region” of about 100 to 110 or more amino acids primarily responsible for antigen recognition. The terms variable light chain (VL) and variable heavy chain (VH) refer to these regions of light and heavy chains respectively. Antibodies may be human, humanized or nonhuman. When a specific aspect of the presently disclosed invention refers to or recites an “antibody,” it is envisioned as referring to any of the full-length antibodies or fragments thereof disclosed herein, unless explicitly denoted otherwise.

A “humanized” antibody refers to an antibody in which some, most or all amino acids outside the CDR domains of a non-human antibody are replaced with corresponding amino acids derived from human immunoglobulins. In one embodiment of a humanized form of an antibody, some, most or all of the amino acids outside the CDR domains have been replaced with amino acids from human immunoglobulins, whereas some, most or all amino acids within one or more CDR regions are unchanged. Small additions, deletions, insertions, substitutions or modifications of amino acids are permissible as long as they do not abrogate the ability of the antibody to bind to a particular antigen. A “humanized” antibody retains an antigenic specificity similar to that of the original antibody.

A “chimeric antibody” refers to an antibody in which the variable regions are derived from one species and the constant regions are derived from another species, such as an antibody in which the variable regions are derived from a mouse antibody and the constant regions are derived from a human antibody.

A “complementarity-determining region”, or “CDR”, refers to amino acid sequences that, together, define the binding affinity and specificity of the variable region of a native immunoglobulin binding site. There are three CDRs in each of the light and heavy chains of an antibody.

A “framework region”, or “FR”, refers to amino acid sequences interposed between CDRs, typically conserved, that act as the scaffold between the CDRs.

A “constant region” refers to the portion of an antibody molecule that is consistent for a class of antibodies and is defined by the type of light and heavy chains. For example, a light chain constant region can be of the kappa or lambda chain type and a heavy chain constant region can be of one of the five chain isotypes: alpha, delta, epsilon, gamma or mu. This constant region, in general, can confer effector functions exhibited by the antibodies. Heavy chains of various subclasses (such as the IgG subclass of heavy chains) are mainly responsible for different effector functions.

As used herein, a HER3 targeting agent may, for example, be an antibody as defined herein, e.g., full length antibody, minibody, or nanobody, that binds to any available epitope of HER3, such as human HER3, with a high immunoreactivity.

As used herein, “Immunoreactivity” refers to a measure of the ability of an immunoglobulin to recognize and bind to a specific antigen. “Specific binding” or “specifically binds” or “binds” refers to an antibody binding to an antigen or an epitope within the antigen with greater affinity than for other antigens, for example, within a relevant context such as within the body of a mammalian subject such as a human patient. An antibody, which may be embodied in or used in the various aspects of the invention, may for example bind to the antigen or the epitope within the antigen with an equilibrium dissociation constant (K_{D}) of about 1×10⁻⁸ M or less, for example about 1×10⁻⁹ M or less, about 1×10⁻¹⁰ M or less, about 1×10⁻¹¹ M or less, or about 1×10⁻¹² M or less, typically with the K_{D} that is at least one hundred fold less than its K_{D} for binding to a nonspecific antigen (e.g., BSA, casein). The dissociation constant may be measured using standard procedures. Antibodies that specifically bind to the antigen or the epitope within the antigen may, however, have cross-reactivity to other related antigens, for example to the same antigen from other species (homologs), such as human or monkey, for example Macaca fascicularis (cynomolgus, cyno), Pan troglodytes (chimpanzee, chimp) or Callithrix jacchus (common marmoset, marmoset).

An “epitope” refers to the target molecule site (e.g., at least a portion of an antigen) that is capable of being recognized by, and bound by, a targeting agent such as an antibody, antibody fragment, Fab fragment, or aptamer. For a protein antigen, for example, this may refer to the region of the protein (i.e., amino acids, and particularly their side chains) that is bound by the antibody. Overlapping epitopes include at least one to five common amino acid residues. Methods of identifying epitopes of antibodies are known to those skilled in the art and include, for example, those described in Antibodies, A Laboratory Manual, Cold Spring Harbor Laboratory, Ed Harlow and David Lane (1988).

Radiolabeled HER3 targeting agents as disclosed herein may be used to treat HER3-positive, i.e., HER3-expressing, cancers or precancerous conditions, such as solid tumors. By “HER3-positive” or “HER3-expressing” it is meant that at least some of the cancer cells within a patient, such as within a tumor, express or over-express HER3.

As used herein, the terms “proliferative disorder” and “cancer” may be used interchangeably and may include, without limitation, a solid cancer (e.g., a tumor) and precancerous proliferative disorders. “Solid cancers” that may be treated by the various aspects of the invention and which may be HER3-positive include, without limitation, bone cancer, pancreatic cancer, skin cancer, cancer of the head or neck, cutaneous or intraocular malignant melanoma, uterine cancer, ovarian cancer, prostate cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, pediatric tumors, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, environmentally-induced cancers including those induced by asbestos. Such cancers may be metastatic or non-metastatic.

According to certain aspects, the solid cancer which may be treated by the various aspects of the invention and which may be HER3-positive, may be breast cancer such as tamoxifen-sensitive breast cancer, tamoxifen-resistant breast cancer, HER2-positive breast cancer, HER2-negative breast cancer, or triple negative breast cancer (TNBC), gastric cancer, bladder cancer, cervical cancer, endometrial cancer, skin cancer such as melanoma, stomach cancer, testicular cancer, esophageal cancer, bronchioloalveolar cancer, prostate cancer such as castration resistant prostate cancer (CRPC), colorectal cancer, ovarian cancer, cervical epidermoid cancer, liver cancer such as hepatocellular carcinoma (HCC) or cholangiocarcinoma, pancreatic cancer, lung cancer such as non-small cell lung carcinoma (NSCLC), renal cancer, head and neck cancer such as head and neck squamous cell cancer, a carcinoma, a sarcoma, or any combination thereof. Such cancers may be metastatic or non-metastatic.

According to certain aspects, the HER3 targeting agent may be labeled with a radioisotope/radionuclide. As used herein, a “radioisotope” or “radionuclide” can be an alpha-emitting isotope, a beta-emitting isotope, and/or a gamma-emitting isotope. Exemplary radionuclides that may be used to label HER3 targeting agents or other targeting agents include the following: ¹³¹I, ¹²⁵I, ¹²³I, ⁹⁰Y, ¹⁷⁷Lu, ¹⁸⁶Re, ¹⁸⁸Re, ⁸⁹Sr, ¹⁵³Sm, ³²P, ²²⁵Ac, ²¹³Bi, ²¹³Po, ²¹¹At, ²¹²Bi, ²¹³Bi, ²²³Ra, ²²⁷Th, ¹⁴⁹Tb, ¹³⁷Cs, ²¹²Pd, ¹⁰³Pd, ¹³⁴Ce, ⁴³Sc, ⁴⁴Sc, ⁴⁷Sc, ⁵⁵Co, ⁶⁰Cu, ⁶¹Cu, ⁶²Cu, ⁶⁴Cu, ⁶⁷Cu, ⁶⁶Ga, ⁶⁷Ga, ⁶⁸Ga, ⁸²Rb, ⁸⁶Y, ⁸⁷Y, ⁸⁹Zr, ⁹⁷Ru, ¹⁰⁵Ph, ¹⁰⁹Pd, ¹¹¹In, ^{117m}Sn, ¹⁴⁹Pm, ¹⁴⁹Tb, ¹⁵³Sm, ¹⁷⁷Lu, ¹⁹⁹Au, ²⁰¹Tl, and ²⁰³Pb. Methods for affixing a radioisotope to a protein such as an antibody or antibody fragment (i.e., “labeling” the protein with the radioisotope) are well known in the art. Specific compositions and methods for labeling are described, for example, in International Publication No. WO 2017/155937 and US Provisional Patent Application Nos. 63/042,651 filed Dec. 9, 2019 and 63/119,093 filed Nov. 30, 2020 titled “Compositions and methods for preparation of site-specific radioconjugates,” each of which is incorporated by reference herein. HER3 targeting agents and other targeting agents containing one or more cysteine residues, such as peptides, proteins, antibodies and protein antibody mimetics may, for example, be chemically conjugated to any of the chelator-bearing, such as DOTA-bearing, stable linkers disclosed in U.S. Pat. No. 11,000,604 titled “Reagent for site-selective bioconjugation of proteins or antibodies” for radionuclide labeling.

According to certain aspects, the HER3 targeting agent may be an antibody, peptide, or small molecule radiolabeled with ²²⁵Ac (“²²⁵Ac-labeled”), and the effective amount may, for example, be at or below 50.0 μCi/kg (i.e., where the amount of ²²⁵Ac administered to the subject delivers a radiation dose of below 50.0 Ci per kilogram of subject's body weight). According to certain aspects, when the HER3 targeting agent is ²²⁵Ac-labeled, the effective amount is below 50 μCi/kg, 40 μCi/kg, 30 μCi/kg, 20 μCi/kg, 10 μCi/kg, 5 μCi/kg, 4 μCi/kg, 3 μCi/kg, 2 μCi/kg, 1 μCi/kg, or 0.5 μCi/kg. According to certain aspects, when the HER3 targeting agent is ²²⁵Ac-labeled, the effective amount is at least 0.05 μCi/kg, or 0.1 μCi/kg, 0.2 μCi/kg, 0.3 μCi/kg, 0.4 μCi/kg, 0.5 μCi/kg, 1 μCi/kg, 2 μCi/kg, 3 μCi/kg, 4 μCi/kg, 5 μCi/kg, 6 μCi/kg, 7 μCi/kg, 8 μCi/kg, 9 μCi/kg, 10 μCi/kg, 12 μCi/kg, 14 μCi/kg, 15 μCi/kg, 16 μCi/kg, 18 μCi/kg, 20 μCi/kg, μCi/kg, or 40 μCi/kg. According to certain aspects, the ²²⁵Ac-labeled antibody may be administered at a dose that includes any combination of upper and lower limits as described herein, such as from at least 0.1 μCi/kg to at or below 5 μCi/kg, or from at least 5 μCi/kg to at or below 20 μCi/kg.

According to certain aspects, the HER3 targeting agent may be an antibody, peptide, or small molecule that is ²²⁵Ac-labeled, and the effective amount may be at or below 2 mCi (i.e., wherein the ²²⁵Ac is administered to the subject in a non-weight-based dosage). According to certain aspects, the effective dose of the ²²⁵Ac-labeled HER3 targeting agent may be at or below 1 mCi, such as 0.9 mCi, 0.8 mCi, 0.7 mCi, 0.6 mCi, 0.5 mCi, 0.4 mCi, 0.3 mCi, 0.2 mCi, 0.1 mCi, 90 μCi, 80 μCi, 70 μCi, 60 μCi, 50 μCi, 40 μCi, 30 μCi, 20 μCi, 10 μCi, or 5 μCi. The effective amount of ²²⁵Ac-labeled HER3 targeting agent may be at least 2 μCi, such as at least 5 Ci, 10 μCi, 20 μCi, 30 μCi, 40 μCi, 50 μCi, 60 μCi, 70 μCi, 80 μCi, 90 μCi, 100 μCi, 200 μCi, 300 μCi, 400 μCi, 500 μCi, 600 μCi, 700 μCi, 800 μCi, 900 μCi, 1 mCi, 1.1 mCi, 1.2 mCi, 1.3 mCi, 1.4 mCi, or 1.5 mCi. According to certain aspects, the ²²⁵Ac-labeled HER3 targeting agent may be administered at a dose that includes any combination of upper and lower limits as described herein, such as from at least 2 μCi to at or below 1 mCi, or from at least 2 μCi to at or below 250 μCi, or from 75 μCi to at or below 400 μCi.

According to certain aspects, the ²²⁵Ac-labeled HER3 targeting agent includes a single dose that delivers less than 12Gy, or less than 8 Gy, or less than 6 Gy, or less than 4 Gy, or less than 2 Gy, such as doses of 2 Gy to 8 Gy, to the subject, such as predominantly to the targeted solid tumor.

According to certain aspects, the HER3 targeting agent may be an antibody, peptide, or small molecule radiolabeled with ¹⁷⁷Lu (“¹⁷⁷Lu-labeled”), and the effective amount may be, for example, at or below 1 mCi/kg (i.e., where the amount of ¹⁷⁷Lu-labeled antibody administered to the subject delivers a radiation dose of below 1000 μCi per kilogram of subject's body weight). According to certain aspects, when the antibody is ¹⁷⁷Lu-labeled, the effective amount is at or below 900 μCi/kg, 800 μCi/kg, 700 μCi/kg, 600 μCi/kg, 500 μCi/kg, 400 μCi/kg, 300 μCi/kg, 200 μCi/kg, 150 μCi/kg, 100 μCi/kg, 80 μCi/kg, 60 μCi/kg, 50 μCi/kg, 40 μCi/kg, 30 μCi/kg, 20 μCi/kg, 10 μCi/kg, 5 μCi/kg, or 1 μCi/kg. According to certain aspects, the effective amount of the ¹⁷⁷Lu-labeled antibody is at least 1 μCi/kg, 2.5 μCi/kg, 5 μCi/kg, 10 μCi/kg, 20 μCi/kg, 30 μCi/kg, 40 μCi/kg, 50 μCi/kg, 60 μCi/kg, 70 μCi/kg, 80 μCi/kg, 90 μCi/kg, 100 μCi/kg, 150 μCi/kg, 200 μCi/kg, 250 μCi/kg, 300 μCi/kg, 350 μCi/kg, 400 μCi/kg or 450 μCi/kg. According to certain aspects, an ¹⁷⁷Lu-labeled antibody may be administered at a dose that includes any combination of upper and lower limits as described herein, such as from at least 5 mCi/kg to at or below 50 μCi/kg, or from at least 50 mCi/kg to at or below 500 μCi/kg.

According to certain aspects, the HER3 targeting agent may be an antibody that is ¹⁷⁷Lu-labeled, and the effective amount may be at or below 45 mCi, such as at or below 40 mCi, 30 mCi, 20 mCi, 10 mCi, 5 mCi, 3.0 mCi, 2.0 mCi, 1.0 mCi, 800 μCi, 600 μCi, 400 μCi, 200 μCi, 100 μCi, or 50 μCi. The effective amount of ¹⁷⁷Lu-labeled HER3 targeting agent may be at or at least 10 μCi, such as at or at least 25 μCi, 50 μCi, 100 μCi, 200 μCi, 300 μCi, 400 μCi, 500 μCi, 600 μCi, 700 μCi, 800 μCi, 900 μCi, 1 mCi, 2 mCi, 3 mCi, 4 mCi, 5 mCi, 10 mCi, 15 mCi, 20 mCi, 25 mCi, 30 mCi. According to certain aspects, an ¹⁷⁷Lu-labeled antibody may be administered at a dose that includes any combination of upper and lower limits as described herein, such as from at least 10 mCi to at or below 30 mCi, or from at least 100 Ci to at or below 3 mCi, or from 3 mCi to at or below 30 mCi.

According to certain aspects, the HER3 targeting agent may be an antibody, peptide, or small molecule radiolabeled with ¹³¹I (“¹³¹I-labeled”), and the effective amount may be at or below, for example, 1200 mCi (i.e., where the amount of ¹³¹I administered to the subject delivers a total body radiation dose of below 1200 mCi in a non-weight-based dose). at or According to certain aspects, the effective amount of the ¹³¹I-labeled targeting agent may be at or below 200 mCi, such as at or below 190 mCi, 180 mCi, 170 mCi, 160 mCi, 150 mCi, 140 mCi, 130 mCi, 120 mCi, 110 mCi, 100 mCi, 90 mCi, 80 mCi, 70 mCi, 60 mCi, or 50 mCi. According to certain aspects, the effective amount of the ¹³¹I-labeled targeting agent may be equal to or at least 1 mCi, such as equal to or at least 2 mCi, 3 mCi, 4 mCi, 5 mCi, 6 mCi, 7 mCi, 8 mCi, 9 mCi, 10 mCi, 20 mCi, 30 mCi, 40 mCi, 50 mCi, 60 mCi, 70 mCi, 80 mCi, 90 mCi, 100 mCi, 110 mCi, 120 mCi, 130 mCi, 140 mCi, 150 mCi, 160 mCi, 170 mCi, 180 mCi, 190 mCi, 200 mCi, 250 mCi, 300 mCi, 350 mCi, 400 mCi, 450 mCi, 500 mCi. According to certain aspects, an ¹³¹I-labeled targeting agent may be administered at a dose that includes any combination of upper and lower limits as described herein, such as from at least 1 mCi to at or below 100 mCi, or at least 10 mCi to at or below 200 mCi.

While select radionuclides are discussed in detail herein, any, such as any disclosed herein, may be used for radiolabeled targeting agents, such as a radiolabeled HER3 targeting agent as disclosed herein.

As used herein, a composition including a HER3 targeting agent includes a “patient specific composition” that includes both a radionuclide labeled portion and an unlabeled portion. According to certain aspects of the present invention, when the HER3 targeting agent is labeled with a radioisotope, the majority of the targeting agent (antibody, antibody fragment, etc.) administered to a patient may consist of unlabeled targeting agent, with the minority being the radiolabeled targeting agent. The ratio of labeled to non-labeled targeting agent can be adjusted using known methods. According to certain aspects of the present invention, the patient specific composition may include the HER3 targeting agent in a ratio of labeled:unlabeled HER3 targeting agent of from about 0.01:10 to 1:1, such as 0.1:10 to 1:1 labeled:unlabeled.

Accordingly to certain aspects of the present invention, the HER3 targeting agent may be provided in a total protein or peptide amount of up to or equal to 100 mg, such as up to or equal to 60 mg, such as 5 mg to 45 mg, or a total protein amount of from 0.001 mg/kg patient weight to 3.0 mg/kg patient weight, such as from 0.005 mg/kg patient weight to 2.0 mg/kg patient weight, or from 0.01 mg/kg patient weight to 1 mg/kg patient weight, or from 0.1 mg/kg patient weight to 0.6 mg/kg patient weight, or 0.3 mg/kg patient weight, or 0.4 mg/kg patient weight, or 0.5 mg/kg patient weight, or 0.6 mg/kg patient weight.

The inventive combination of a radiolabeled fraction and an unlabeled fraction of the antibody or other targeting agent allows the composition to be tailored to a specific patient, wherein each of the radiation dose and the protein dose of the antibody or other targeting agent are personalized to that patient based on at least one patient-specific parameter. As such, each vial of the composition may be made for a specific patient, where the entire content of the vial is delivered to that patient in a single dose. When a treatment regime calls for multiple doses, each dose may be formulated as a patient specific dose in a vial to be administered to the patient as a “single dose” (i.e., full contents of the vial administered at one time). The subsequent dose may be formulated in a similar manner, such that each dose in the regime provides a patient specific dose in a single dose container. One of the advantages of such a composition is that there will be no left-over radiation that would need to be discarded or handled by the medical personnel, e.g., no dilution, or other manipulation to obtain a dose for the patient. When provided in a single dose container, the container may simply be placed in-line in an infusion tubing set for infusion to the patient. Moreover, the volume can be standardized so that there is a greatly reduced possibility of medical error (i.e., delivery of an incorrect dose, as the entire volume of the composition is to be administered in one infusion).

Thus, according to certain aspects, the HER3 targeting agent may be provided as a single dose composition which may be tailored to a specific patient, wherein the amount of radiolabeled and unlabeled HER3 targeting agent in the composition may depend on one or more of a patient weight, age, gender, disease state and/or health status, such as detailed in International Publication No. WO 2016/187514 and U.S. Pat. No. 10,736,975. According to certain aspects, the HER3 targeting agent may be provided as a multi-dose therapeutic, wherein each dose in the treatment regime is provided as a patient specific composition. The patient-specific composition includes radiolabeled and unlabeled HER3 targeting agents, wherein the amounts of each depend on one or more of patient weight, age, gender, disease state, and/or health status.

As used herein, the terms “subject” and “patient” are interchangeable and include, without limitation, a mammal such as a human, a non-human primate, a dog, a cat, a horse, a sheep, a goat, a cow, a rabbit, a pig, a rat and a mouse. Where the subject is human, the subject may be of any age. For example, the subject can be 60 years or older, 65 or older, 70 or older, 75 or older, 80 or older, 85 or older, or 90 or older. Alternatively, the subject can be 50 years or younger, 45 or younger, 40 or younger, 35 or younger, 30 or younger, 25 or younger, or 20 or younger. For a human subject afflicted with cancer, the subject can be newly diagnosed, or relapsed and/or refractory, or in remission.

As used herein, “treating” a subject afflicted with a cancer shall include, without limitation, (i) slowing, stopping or reversing the cancer's progression, (ii) slowing, stopping or reversing the progression of the cancer's symptoms, (iii) reducing the likelihood of the cancer's recurrence, and/or (iv) reducing the likelihood that the cancer's symptoms will recur. According to certain preferred aspects, treating a subject afflicted with a cancer means (i) reversing the cancer's progression, ideally to the point of eliminating the cancer, and/or (ii) reversing the progression of the cancer's symptoms, ideally to the point of eliminating the symptoms, and/or (iii) reducing or eliminating the likelihood of relapse (i.e., consolidation, which ideally results in the destruction of any remaining cancer cells).

“Chemotherapeutic”, in the context of this invention, shall mean a chemical compound which inhibits or kills growing cells and which can be used or is approved for use in the treatment of cancer. Exemplary chemotherapeutic agents include cytostatic agents which prevent, disturb, disrupt or delay cell division at the level of nuclear division or cell plasma division. Such agents may stabilize microtubules, such as taxanes, in particular docetaxel or paclitaxel, and epothilones, in particular epothilone A, B, C, D, E, and F, or may destabilize microtubules such as vinca alkaloids, in particular vinblastine, vincristine, vindesine, vinflunine, and vinorelbine. Exemplary chemotherapeutics also include radiosensitizers that may synergize with the radiolabeled HER3, such as temozolomide, cisplatin, and/or fluorouracil.

“Therapeutically effective amount” or “effective amount” refers to an amount effective, at dosages and for periods of time necessary, to achieve a desired therapeutic result. A therapeutically effective amount may vary according to factors such as the disease state, age, sex, and weight of the individual, and the ability of a therapeutic or a combination of therapeutics to elicit a desired response in the individual. Exemplary indicators of an effective therapeutic or combination of therapeutics include, for example, improved well-being of the patient, reduction in a tumor burden, arrested or slowed growth of a tumor, and/or absence of metastasis of cancer cells to other locations in the body. According to certain aspects, “therapeutically effective amount” or “effective amount” refers to an amount of the radiolabeled HER3 targeting agent that may deplete or cause a reduction in the overall number of cells expressing HER3 and/or that may inhibit growth of cells expressing HER3, when used alone or in combination or conjunction with other agents and/treatment modalities.

As used herein, “depleting”, with respect to cells expressing HER3, shall mean to lower the population of at least one type of cells that express or overexpress HER3 (e.g., HER3-positive cells in a solid tumor or circulating in a subject's blood). According to certain aspects of this invention, a decrease is determined by comparison of the numbers of HER3-positive cells in the subject's blood or in a tissue biopsy, such as from the solid tumor, before and after initiation of treatment with the HER3 targeting agent. As such, and by way of example, a subject's HER3-positive cells may be considered to be depleted if the population is lowered, such as by at least 10%, 20%, 30%, 40%, 50%, 60%, 70%, 80%, 90% or 99%.

“Inhibits growth” refers to a measurable decrease or delay in the growth of a malignant cell or tissue (e.g., tumor) in vitro or in vivo when contacted with a therapeutic or a combination of therapeutics or drugs, when compared to the decrease or delay in the growth of the same cells or tissue in the absence of the therapeutic or the combination of therapeutic drugs. Inhibition of growth of a malignant cell or tissue in vitro or in vivo may be at least about 10%, 20%, 30%, 40%, 50%, 60%, 70%, 80%, 90%, 95%, 96%, 97%, 98%, 99%, or 100%.

The term “immune checkpoint therapy” refers to a molecule capable of modulating the function of an immune checkpoint protein in a positive or negative way in the furtherance of immune response against cancer cells. The term “immune checkpoint” refers to a protein directly or indirectly involved in an immune pathway that under normal physiological conditions acts to prevent uncontrolled immune reactions and thus for the maintenance of self-tolerance and/or tissue protection.

In the context of the present invention, an immune checkpoint therapy encompasses therapies such as antibodies capable of down-regulating at least partially the function of an inhibitory immune checkpoint (antagonist) and/or up-regulating at least partially the function of a stimulatory immune checkpoint (agonist). As example, an immune checkpoint therapy may refer to an antibody against an immune checkpoint inhibitor (ICI) that may be upregulated in certain cancers, and thus may inhibit the function of the ICI.

The term “DDRi” refers to an inhibitor of a DNA damage response pathway protein, of which a PARPi is an example. The term “PARPi” refers to an inhibitor of poly(ADP-ribose) polymerase. In the context of the present invention, the term PARPi encompasses molecules that may bind to and inhibitor the function of poly(ADP-ribose) polymerase, such as antibodies, peptides, or small molecules.

The term “CD47 blockade” refers to an agent that prevents CD47 binding to SIRPα, such as blocking agents that bind to either of CD47 or SIRPα, or those that modulate expression of CD47 or SIRPα, or those that otherwise inhibit the CD47/SIRPα axis. Without limitation, CD47 blockades encompass at least antibodies that bind to CD47 such as magrolimab, lemzoparlimab, and AO-176, SIRPα fusion proteins such as TTI-621 and TTI-622, agents that modulate the expression of CD47 and/or SIRPα, such as phosphorodiamidate morpholino oligomers (PMO) that block translation of CD47, and small molecule agents such as RRx-001.

As used herein, administering to a subject one or more additional therapies, such as one or more of an immune checkpoint therapy and/or DDRi and/or CD47 blockade and/or radiosensitizer and/or VEGF inhibitor and/or VEGFR inhibitor “in conjunction with” a radiolabeled HER3 targeting agent means administering the additional therapy before, during and/or after administration of the radiolabeled HER3 targeting agent. This administration includes, without limitation, the following scenarios: (i) the additional therapy is administered first, and the radiolabeled HER3 targeting agent is administered second; (ii) the additional therapy is administered concurrently with the radiolabeled HER3 targeting agent (e.g., the DDRi is administered orally once per day for n days, and the HER3 targeting agent is administered intravenously in a single dose on one of days 2 through n−1 of the DDRi regimen); (iii) the additional therapy is administered concurrently with the radiolabeled HER3 targeting agent (e.g., the DDRi is administered orally for a duration of greater than one month, such as orally once per day for 35 days, 42 days, 49 days, or a longer period during which the cancer being treated does not progress and during which the DDRi does not cause unacceptable toxicity, and the radiolabeled HER3 targeting agent is administered intravenously in a single dose on a day within the first month of the DDRi regimen); and (iv) the radiolabeled HER3 targeting agent is administered first (e.g., intravenously in a single dose or a plurality of doses over a period of weeks), and the additional therapy is administered second (e.g., the DDRi is administered orally once per day for 21 days, 28 days, 35 days, 42 days, 49 days, or a longer period during which the cancer being treated does not progress and during which the DDRi does not cause unacceptable toxicity). Additional permutations that would be obvious to one of skill in the art are possible and within the scope of the presently claimed invention.

An “article of manufacture” indicates a package containing materials useful for the treatment, prevention and/or diagnosis of the disorders described herein. The article of manufacture may include a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper pierceable by a hypodermic injection needle). At least one active agent in the composition may be a radiolabeled HER3 targeting agent according to aspects of the presently disclosed invention.

A “label” or “package insert” is used to refer to instructions customarily included in commercial packages of therapeutic products that contain information about the indications, usage, dosage, administration, combination therapy, contraindications and/or warnings concerning the use of such therapeutic products. As used herein, a label may indicate that the composition is used for treating a HER3-positive cancer and may optionally indicate administration routes and/or methods. Moreover, the article of manufacture may include (a) a first container with a composition contained therein, wherein the composition includes HER3 targeting agent; and (b) a second container with a composition contained therein, wherein the composition includes a further cytotoxic or otherwise therapeutic agent according to aspects of the presently disclosed invention. Alternatively, or additionally, the article of manufacture may further include a second (or third) container including a pharmaceutically acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

Throughout this application, various patents, patent applications and other publications are cited, each of which is hereby incorporated by reference in its entirety.

Unless otherwise defined, all technical and scientific terms used herein have the same meaning as commonly understood by one of ordinary skill in the art to which the present invention belongs. Although methods and materials similar or equivalent to those described herein can be used in the practice or testing described herein, suitable methods and materials are described below.

### Experimental Results

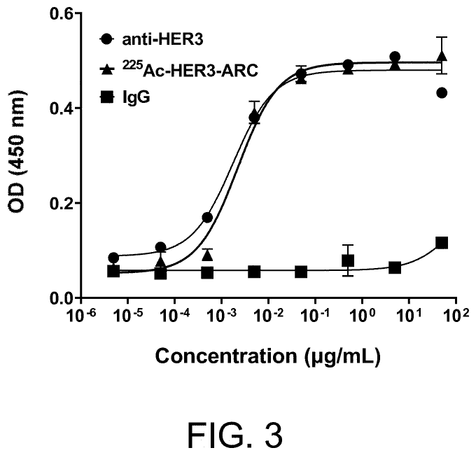

An anti-HER3 IgG monoclonal antibody consisting of heavy chain SEQ ID NO:77 and light chain SEQ ID NO:78 was prepared, conjugated to the chelator DOTA using p-SCN-Bn-DOTA and radiolabeled via chelation with Actinium-225 for further investigation as described below in connection with FIGS. 3-11.

FIG. 3 shows ELISA assay binding characteristics of an Ac225 labeled DOTA-conjugated anti-HER3 monoclonal antibody (“HER3-ARC”) versus the unmodified anti-HER3 antibody and a non-specific antibody (IgG), demonstrating that the modifications do not materially affect immune reactivity to HER3.

The binding properties of 225Ac-HER3-ARC were evaluated by ELISA. A 96 well plate was coated with human recombinant HER3 overnight following by incubation of serial dilutions (0-100 μg/ml) of anti-HER3, 225Ac-HER3-ARC and IgG (immunoglobulin 1, nonpecific IgG1 control) for 1 h at room temperature. A secondary antibody (Goat Anti-human IgG F(ab′)20-HRP) was added and incubated for 30 min on ice followed by color development using HCl 1M for 10 min. The sample absorbance was measured at 450 nm. ²²⁵Ac-HER3-ARC showed similar binding properties to those of the native antibody by ELISA (HER3-ARC: EC₅₀=0.0017 μg/ml, HER3 EC₅₀=0.0022 μg/ml).

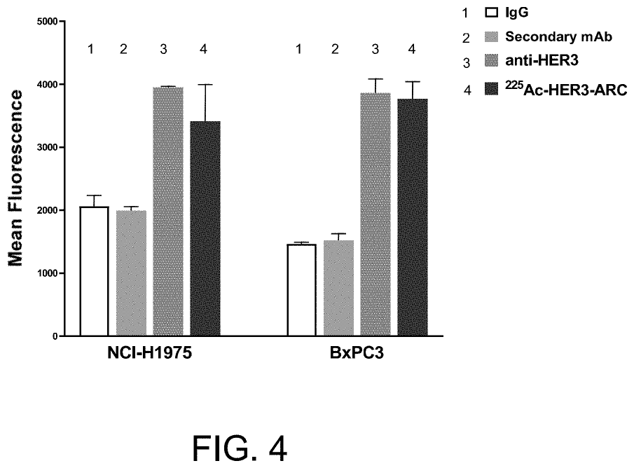

FIG. 4 is a graph showing the results of flow cytometry assays examining the binding of the 225Ac-HER3-ARC, the unmodified anti-HER3 mAb, non-specific antibody control (IgG), and secondary antibody only control to HER3-positive NCI-H1975 cells (human lung adenocarcinoma, NSCLC) and BxPC-3 cells (human pancreatic adenocarcinoma).

The binding properties of 225Ac-HER3-ARC were evaluated by flow cytometry in HER3+ cells (NCI-H1975 and BxPC3). Solutions (100 μg/ml) of anti-HER3, 225Ac-HER3-ARC and IgG (immunoglobulin 1, nonspecific IgG1) were added to HER+ cells and incubated for 1 h at room temperature. A PE labeled secondary antibody was added and incubated for 30 min on ice. Sample fluorescence was measured using a flow cytometer. The binding properties of 225Ac-HER3-ARC to HER3+ positive cell lines resembled those of the unmodified anti-HER3 mAb.

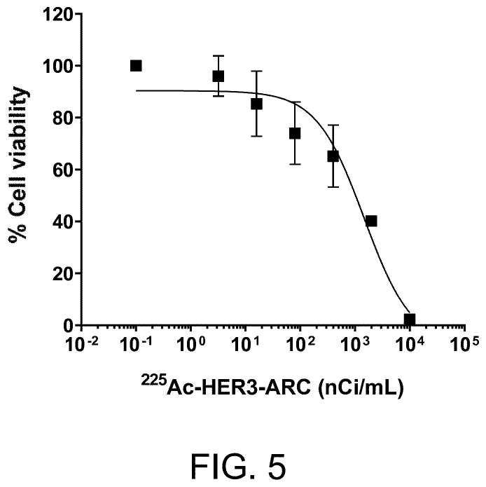

FIG. 5 is a graph showing the in vitro cytotoxic effect of 225Ac-HER3-ARC to HER3-positive cell line NCI-H1975 as a function of radiation dose.

The cytotoxic effects of 225Ac-HER3-ARC to HER3+ cell line NCI-H1975 were evaluated in a colorimetric assay using CellTiter 96® AQueous Non-Radioactive Cell Proliferation Assay (MTS). NCI-H1975 cells were incubated with 225Ac-HER3-ARC for 24 h at 37° C. Unbound 225Ac-HER3-ARC was then removed, and cells were cultured for 72 h at 37° C. Absorbance at 490 nm was measured and % of cell viability calculated. 225Ac-HER3-ARC showed potent in vitro cytotoxicity against HER3+ cell line NCI-H1975.

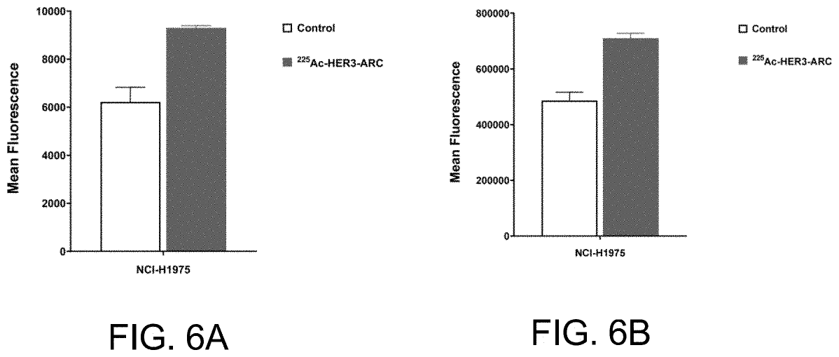

FIG. 6A is a graph showing that 225Ac-HER3-ARC upregulates cell surface calreticulin (CRT) in NCI-H1975 cells and FIG. 6B is a graph showing that 225Ac-HER3-ARC upregulates CD47 on NCI-H1975 cells.

The effect of 225Ac-HER3-ARC on cell surface expression of calreticulin (CRT) and CD47 by HER3+ cell line NCI-H1975 was examined using flow cytometry. Cells were treated with 225Ac-HER3-ARC (100 nCi/ml) or PBS (control) for 72 h. Following treatment, cells were stained for CRT and CD47. The results demonstrate that each of CRT (FIG. 6A) and CD47 (FIG. 6B) is upregulated by 225Ac-HER3-ARC in NCI-H1975 cells.

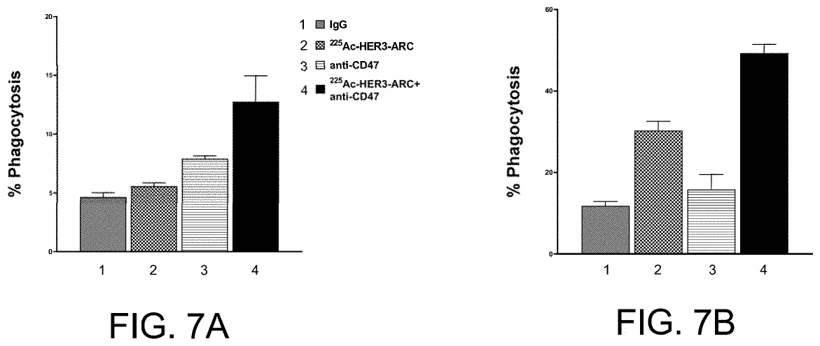

FIG. 7A is a graph showing results of a phagocytosis assay demonstrating that the combination of 225Ac-HER3-ARC and an anti-CD47 blocking antibody enhanced phagocytosis of BxPC-3 cells versus either treatment alone. FIG. 7B is a graph showing results of a phagocytosis assay demonstrating that the combination of 225Ac-HER3-ARC and an anti-CD47 blocking antibody enhanced phagocytosis of NCI-H1975 cells versus either treatment alone. The same key applies for FIGS. 7A and 7B.

The effect of combining 225Ac-HER3-ARC and anti-CD47 on phagocytosis in vitro was evaluated by flow cytometry. BxPC-3 (FIG. 7A) and NCI-H1975 (FIG. 7B) cells were seeded in 6-well plates 24 hr prior to a 24 hr incubation at 37° C. with 225Ac-HER3-ARC. Following 225Ac-HER3-ARC treatment, cells were cultured for 72 hr at 37° C.

BxPC-3 and NCI-H1975 cells were stained with Vybrant DiD cell-labeling solution and treated with anti-human CD47 (Bio X Cell, Cat #BE0019) and mouse IgG1 isotype control (Bio X Cell, Cat #BE0083) for 1 hr at 37° C. Human macrophages were stained with Vybrant DiO cell-labeling solution. Labeled human macrophages and target cells were cocultured for 2 h at 37° C. Phagocytosis was assessed by evaluating the dual labeled cells (DiD+/DiO+).

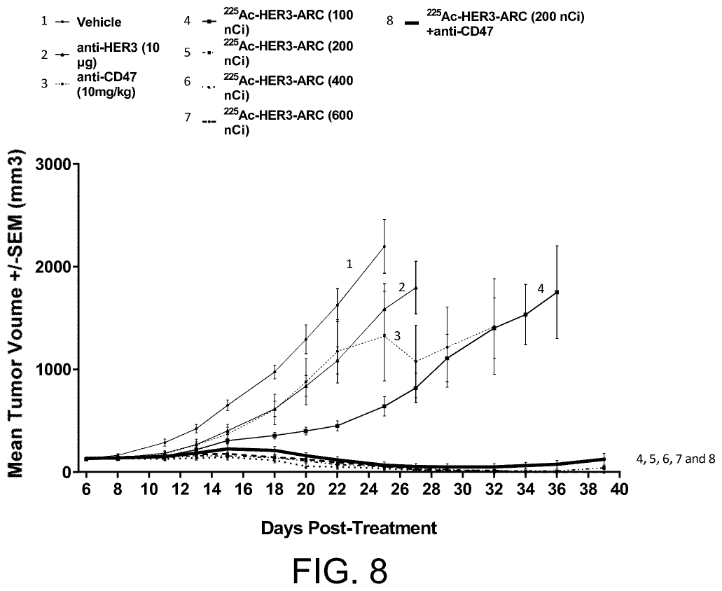

FIG. 8 is graph showing the effects on tumor growth, in a human tumor (NCI-H1975 cell) mouse xenograft model, of a 225Ac-HER3-ARC at different radiation doses (100 nCi, 200 nCi, 400 nCi, 600 nCi) alone and at 200 nCi in combination with an anti-CD47 blocking antibody, of unlabeled anti-HER3 mAb, of anti-CD47 blocking antibody alone, and of vehicle-only control. Notably, tumor growth was almost entirely suppressed by 225Ac-HER-ARC at each of radiation doses 200 nCi, 400 nCi, 600 nCi and by the combination of 225Ac-HER-ARC (200 nCi) with the anti-CD47 mAb.

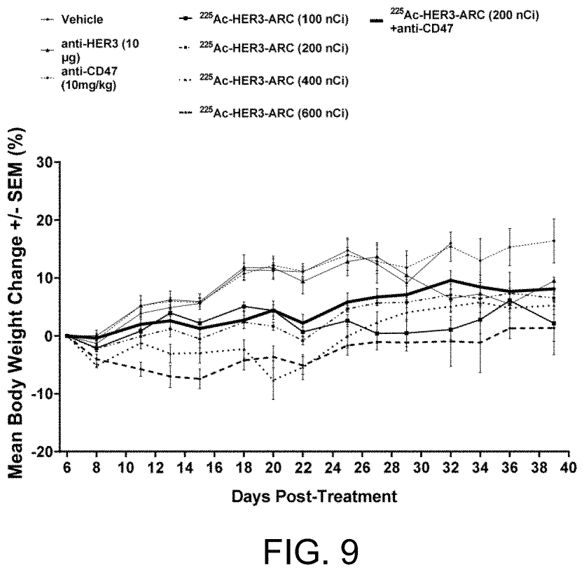

FIG. 9 is a graph showing body weight over time for the subjects of the experiment described in FIG. 8.

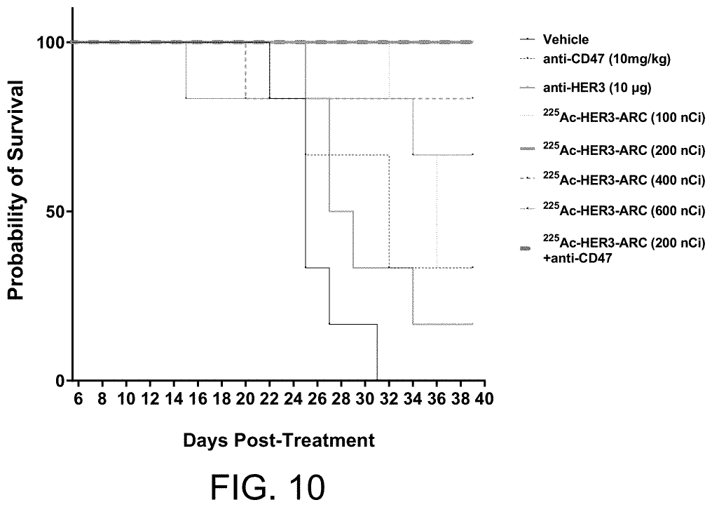

FIG. 10 is a graph showing the probability of survival over time for the experimental group subjects of the experiment described in FIG. 8.

Tumor xenograft studies examining the effect of HER2-ARC treatment alone and in combination with CD47 blockade on HER2-positive tumor growth were also performed. Anti-HER2 mAb Trastuzumab was chemically conjugated to DOTA using p-SCN-Bn-DOTA and labeled, via chelation, with either Actinium-225 or Lutetium-177 for use in these experiments.

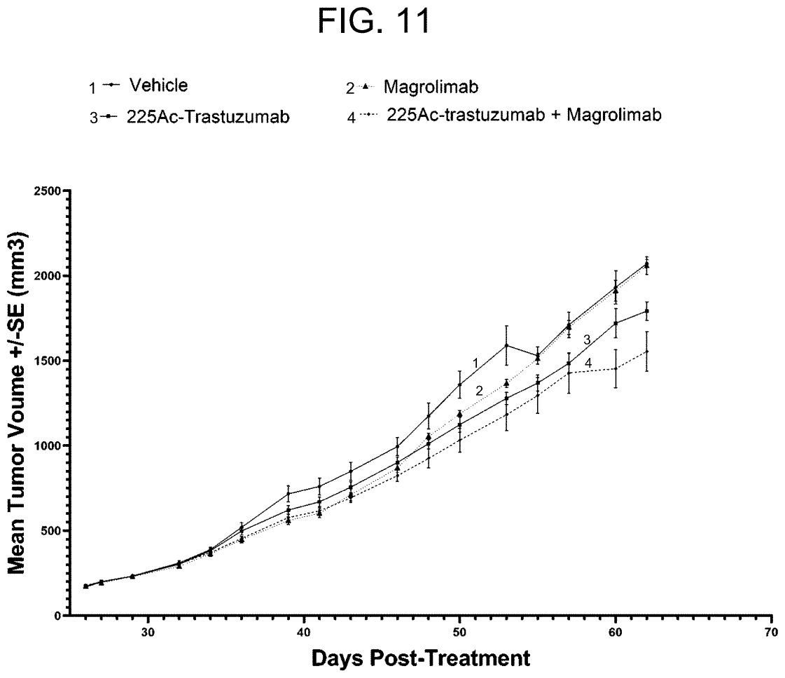

FIG. 11 is a graph showing the comparative effects on tumor growth of vehicle only (control), magrolimab alone (10 mg/kg), 225Ac-trastuzumab alone (0.025 μCi/animal), and the combination of magrolimab (10 mg/kg) and 225Ac-trastuzumab (0.025 μCi/animal), in an NGS mouse xenograft model using the HER2-positive SK-OV3 human ovarian cancer cell line. Each cohort consisted of eight animals.

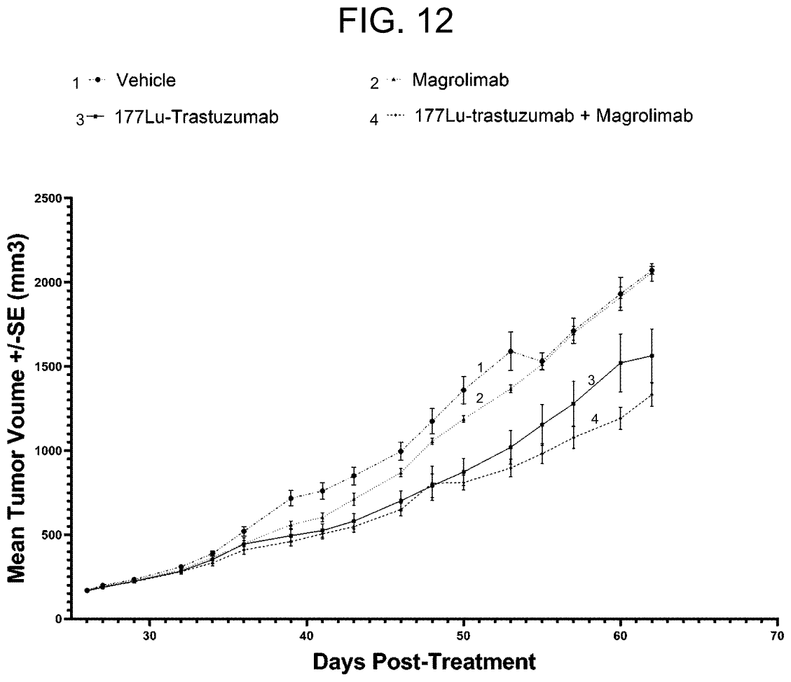

FIG. 12 is a graph showing the comparative effects on tumor growth of vehicle only (control), magrolimab alone (10 mg/kg), 177Lu-trastuzumab alone (25 μCi/animal), and the combination of magrolimab (10 mg/kg) and 177Lu-trastuzumab (25 μCi/animal), in an NGS mouse xenograft model using the HER2-positive SK-OV3 human ovarian cancer cell line. Each cohort consisted of eight animals.

### Aspects of the Invention

It is well documented in both preclinical and clinical studies that levels of HER3 can become downregulated following administration of a HER3-targeting antibody (Mishra, 2018).

In preclinical models with lumretuzumab, there was a dose-dependent (1-10 mg/kg) downregulation of HER3 as measured by both immunohistochemistry and Western blotting (Maneses-Lorenta, 2015; Mirshberger, 2013). The lowest dose of lumretuzumab (0.3 mg/kg) did not result in HER3 target downregulation (Maneses-Lorenta, 2015), and these low levels of lumretuzumab (0.1 mg/kg and 0.3 mg/kg) were ineffective at controlling HER3-expressing tumors (Mirshberger, 2013). In clinical studies with lumretuzumab, downregulation of surface HER3 was observed in serial tumor biopsies in 92% of patients across all dose levels tested (100-2000 mg; Meulendijks, 2016). Additionally, a decrease in total HER3 levels was observed in three out of five paired tumor biopsy samples in patients treated with the HER3-targeting antibody LJM716 at 40 mg/kg (Reynolds, 2017).

While the internalization and degradation of HER3 may be beneficial to reduce phosphorylation of HER3 and subsequent signaling activity, reduction of surface levels of HER3 may impede antibody targeting of tumors. Therefore, if repeat administration of a HER3-targeting antibody is desired or required for efficacy, the administration of a HER3-targeting antibody may result in downregulation of the target and preclude re-dosing. The present inventors have found use of antibody radioconjugates (ARCs) circumvent the problems associated with the dose-dependent downregulation of HER3 as the lower antibody doses useful in therapeutic methods may not cause HER3 downregulation. Accordingly, the present inventors have found that HER3 targeting agents including a radioisotope are effective as diagnostic and therapeutic agents for improved tumor targeting and killing of HER3-expressing cancer cells, such as certain solid tumors. In particular, therapeutic methods that may include multiple doses of a HER3-targeting agent may provide improved tumor targeting and killing without causing a detrimental level of HER3 downregulation.

Thus, according to certain aspects of the presently disclosed invention, therapeutic methods for treating HER3-positive cancers using a radiolabeled HER3 targeting agent are provided. The methods may also include diagnostic steps to determine if and/or to what extent a patient has a HER3-positive cancer and/or the localization of such cancer, for example, by identifying and/or quantifying HER3 positive cells within solid tumors or circulating in a blood sample from the patient.

According to certain aspects, the therapeutic methods include administration of a radiolabeled HER3 targeting agent, such as a radiolabeled antibody, peptide, or small molecule that targets HER3, either alone or in combination with one or more additional therapeutic agents or modalities. According to certain aspects, the additional agent or modality may be any one or more of administration of an immune checkpoint therapy, a DDRi, a CD47 blockade, a chemotherapeutic agent, a small molecule oncology drug, external beam radiation, and brachytherapy.

According to certain aspects, the radiolabeled HER3 targeting agent may be administered to the patient in a patient specific composition in one or more doses.

According to certain aspects, the patient may be monitored at intervals during the therapy for the presence of HER3-positive cells to evaluate the reduction in HER3-positive cells. Detecting a decreased number of the HER3-positive cells after treatment with the HER3 targeting agent, as compared to the number of HER3-positive cells prior to treatment may indicate effectiveness of the HER3 targeting agent in treating a HER3-positive cancer in the mammalian subject.

According to certain aspects, the method of treating cancer includes identifying a patient having a HER3-positive cancer by identifying HER3-positive cells and administering to the patient an effective amount of a HER3 targeting agent, either alone or in combination with an additional method of treatment. According to certain aspects, the additional method of treatment may be any one or more of administration of an immune checkpoint therapy, a DDRi, a CD47 blockade, a chemotherapeutic agent, a small molecule oncology drug, external beam radiation and brachytherapy.

According to certain aspects, the chemotherapeutic agent is a radiosensitizer.

According to certain aspects, the radiolabeled HER3 targeting agent can be administered to a patient that has undergone, such as recently undergone a treatment, such as surgery for treatment of the cancer, such as to remove all or a portion of a solid tumor. Thus, for example, the radiolabeled HER3 targeting agent may be administered perioperatively or post-operatively.

HER3 Targeting Agents

An object of the presently disclosed invention is to provide radiolabeled HER3-, such as human HER3-, targeting agents for diagnostic use and/or for therapeutic use, such as in the diagnosis and/or treatment of HER3-positive cancers. Radiolabeled HER3-targeting agents can effect a therapeutic response via the delivery of DNA-damaging ionizing radiation to cells, for example, alpha-particles that induce double strand DNA breaks and cell death.

Exemplary anti-HER3 antibodies (also referred to as “HER3 antibodies” herein), such as anti-human HER3 antibodies, that that may be radiolabeled and embodied in and/or used in the various aspect of the presently disclosed invention include, without limitation, the following antibodies, and antibodies such as but not limited to immunoglobulins, such as but not limited to IgG, that (i) include the heavy chain variable region of the HER3 antibody or heavy chain, (ii) include 1, 2 or 3 of the heavy chain CDRs (e.g., by the Kabat definition) of the HER3 antibody or heavy chain or those recited, (iii) include the light chain variable region of the HER3 antibody or light chain, and/or (iv) include 1, 2 or 3 of the light chain CDRs (e.g., by the Kabat definition) of the HER3 antibody or light chain or those recited. It should also be understood that where a HER3 antibody heavy chain or HER3 antibody light chain is disclosed that includes an N-terminal leader sequence, also intended to be disclosed for embodiment in and use in the various aspects of the invention are corresponding heavy chains and corresponding light chains that lack the leader sequence.

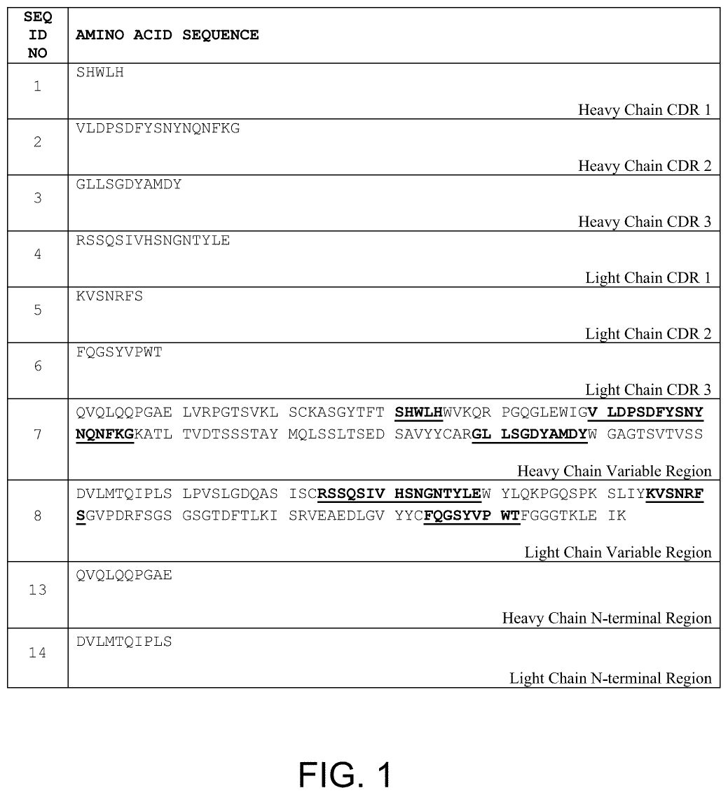

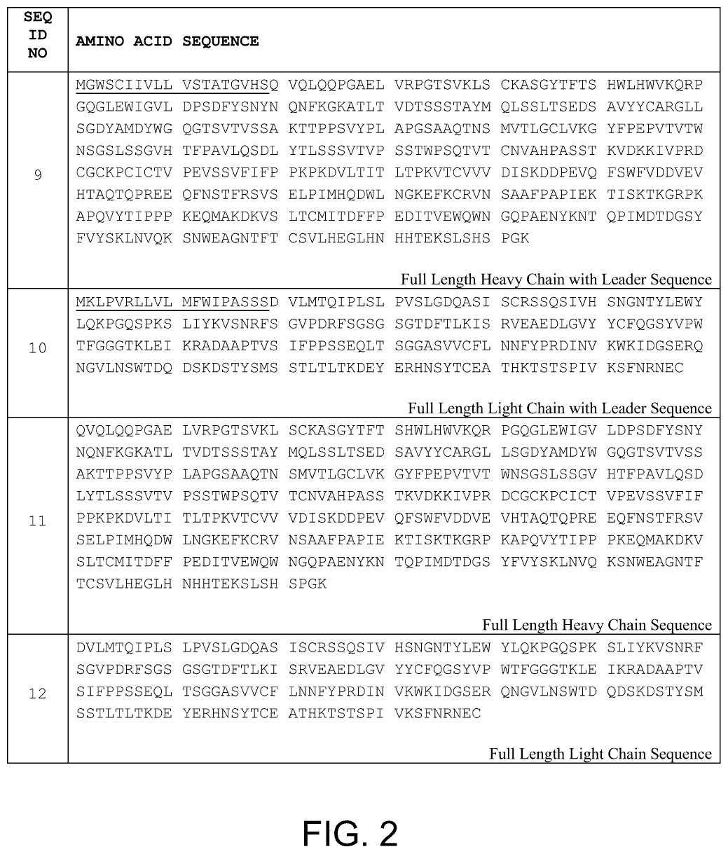

An exemplary HER3 antibody that may be radiolabeled and embodied in and/or used in the presently disclosed invention may, for example, include a murine monoclonal antibody against HER3 including a heavy chain having the amino acid sequence as set forth in SEQ ID NO:9 or 11 and/or a light chain having the amino acid sequence as set forth in SEQ ID NO:10 or 12, or an antibody such as a humanized antibody derived from one or more of said sequences. An exemplary HER3 antibody that may be radiolabeled and embodied in and/or used in the presently disclosed invention may include or a heavy chain with an N-terminal region having the sequence set forth in SEQ ID NO:13 and/or a light chain with an N-terminal region having the sequence as set forth in SEQ ID NO: 14. A HER3 antibody that may be similarly embodied or used in various aspect of the invention may, for example, include the heavy chain variable region having the amino acid sequence as set forth in SEQ ID NO:7, and/or a light chain variable region having an amino acid sequence as set forth in SEQ ID NO:8; and/or a heavy chain including one or more of CDR1, CDR2 and CDR3 having the amino acid sequences respectively set forth in SEQ ID NOS:1-3, and/or a light chain with one or more of the CDR1, CD2 and CDR3 having the amino acid sequences respectively set forth in SEQ ID NOS:4-6. See FIGS. 1 and 2 for a further description of these sequences. A HER3 antibody embodied in and/or used in any of the aspects of the invention may, for example, include any combination of the aforementioned light chain sequences and/or heavy chain sequences.

An exemplary HER3 antibody includes an immunoglobulin heavy chain variable region including a CDR-H1 including SEQ ID NO:15, a CDR-H2 including SEQ ID NO:16, and a CDR-H3 including SEQ ID NO: 17, and/or an immunoglobulin light chain variable region including a CDR-L1 including SEQ ID NO:18, a CDR-L2 including SEQ ID NO:19, and a CDR-L3 including SEQ ID NO:20. An exemplary An exemplary HER3 antibody includes an immunoglobulin heavy chain variable region including SEQ ID NO:21 and/or an immunoglobulin light chain variable region including SEQ ID NO:22. An exemplary HER3 antibody includes an immunoglobulin heavy chain amino acid sequence of SEQ ID NO:23 and/or an immunoglobulin light chain amino acid sequence of SEQ ID NO:24.

An exemplary HER3 antibody includes an immunoglobulin heavy chain variable region including a CDR-H1 including SEQ ID NO:25, a CDR-H2 including SEQ ID NO:26, and a CDR-H3 including SEQ ID NO:27; and/or an immunoglobulin light chain variable region including a CDR-L1 including SEQ ID NO:28, a CDR-L2 including SEQ ID NO:29, and a CDR-L3 including SEQ ID NO:30. An exemplary HER3 antibody includes an immunoglobulin heavy chain variable region including SEQ ID NO:31 and/or an immunoglobulin light chain variable region including SEQ ID NO:32. An exemplary HER3 antibody includes an immunoglobulin heavy chain amino acid sequence of SEQ ID NO:33 and/or an immunoglobulin light chain amino acid sequence of SEQ ID NO:34

An exemplary HER3 antibody includes an immunoglobulin heavy chain variable region including a CDR-H1 including SEQ ID NO:35, a CDR-H2 including SEQ ID NO:36, and a CDR-H3 including SEQ ID NO:37; and/or an immunoglobulin light chain variable region including a CDR-L1 including SEQ ID NO:38, a CDR-L2 including SEQ ID NO:39, and a CDR-L3 including SEQ ID NO:40. An exemplary HER3 antibody includes an immunoglobulin heavy chain variable region including SEQ ID NO:41, and/or an immunoglobulin light chain variable region SEQ ID NO:42. An exemplary HER3 antibody includes an immunoglobulin heavy chain amino acid sequence of SEQ ID NO:43 and an immunoglobulin light chain amino acid sequence of SEQ ID NO:44.

An exemplary HER3 antibody includes an immunoglobulin heavy chain variable region including a CDR-H1 including SEQ ID NO:45, a CDR-H2 including SEQ ID NO:46, and a CDR-H3 including SEQ ID NO:47; and/or an immunoglobulin light chain variable region including a CDR-L1 including SEQ ID NO 48, a CDR-L2 including SEQ ID NO:29, and a CDR-L3 including SEQ ID NO:49. An exemplary HER3 antibody includes an immunoglobulin heavy chain variable region including SEQ ID NO:50 and/or an immunoglobulin light chain variable region including SEQ ID NO:51. An exemplary HER3 antibody includes an immunoglobulin heavy chain amino acid sequence of SEQ ID NO:52 and/or an immunoglobulin light chain amino acid sequence of SEQ ID NO:53.

An exemplary HER3 antibody includes an immunoglobulin heavy chain variable region including a CDR-H1 including SEQ ID NO:54, a CDR-H2 including SEQ ID NO:55, and a CDR-H3 including SEQ ID NO:56; and/or an immunoglobulin light chain variable region including a CDR-L1 including SEQ ID NO:28, a CDR-L2 including SEQ ID NO:29, and a CDR-L3 including SEQ ID NO:30. An exemplary HER3 antibody includes an immunoglobulin heavy chain variable region including SEQ ID NO:57 and/or an immunoglobulin light chain variable region including SEQ ID NO:58. An exemplary HER3 antibody includes an immunoglobulin heavy chain amino acid sequence of SEQ ID NO:59 and/or an immunoglobulin light chain amino acid sequence of SEQ ID NO: 60.

An exemplary HER3 antibody includes an immunoglobulin heavy chain variable region including a CDR-H1 including SEQ ID NO:61, a CDR-H2 including SEQ ID NO:62, and a CDR-H3 including SEQ ID NO:63; and/or an immunoglobulin light chain variable region including a CDR-L1 including SEQ ID NO:64, a CDR-L2 including SEQ ID NO:65, and a CDR-L3 including SEQ ID NO:66. An exemplary HER3 antibody includes an immunoglobulin heavy chain variable region including SEQ ID NO:67, and/or an immunoglobulin light chain variable region including SEQ ID NO:68. An exemplary HER3 antibody includes an immunoglobulin heavy chain amino acid sequence of SEQ ID NO:69 and an immunoglobulin light chain amino acid sequence of SEQ ID NO:70.

An exemplary HER3 antibody includes an immunoglobulin heavy chain variable region including a CDR-H1 including SEQ ID NO:71, a CDR-H2 including SEQ ID NO:72, and a CDR-H3 including SEQ ID NO:66; and/or an immunoglobulin light chain variable region including a CDR-L1 including SEQ ID NO:28, a CDR-L2 including SEQ ID NO:29, and a CDR-L3 including SEQ ID NO:30. An exemplary HER3 antibody includes an immunoglobulin heavy chain variable region including SEQ ID NO:73, and/or an immunoglobulin light chain variable region including SEQ ID NO:74. An exemplary HER3 antibody includes an immunoglobulin heavy chain amino acid sequence of SEQ ID NO:75 and/or an immunoglobulin light chain amino acid sequence of SEQ ID NO:76.

An exemplary HER3 antibody includes an immunoglobulin heavy chain amino acid sequence of SEQ ID NO:77 and/or an immunoglobulin light chain amino acid sequence of SEQ ID NO:78.

An exemplary HER3 antibody includes an immunoglobulin light chain variable region including SEQ ID NOS:86, 87, 88, 89, 90 or 91 and/or a heavy chain variable region including SEQ ID NOS:79, 80, 81, 82, 83, 84 or 85.

An exemplary HER3 antibody includes an immunoglobulin heavy chain sequence including SEQ ID NO:92, 94, 95, 98 or 99 and/or an immunoglobulin light chain sequence including SEQ ID NO:93, 96, 97, 100 or 101.

Exemplary HER3 antibodies also include Barecetamab (ISU104) from Isu Abxis Co and any of the HER3 antibodies disclosed in U.S. Pat. No. 10,413,607.

Exemplary HER3 antibodies also include HMBD-001 (10D1F) from Hummingbird Bioscience Pte. and any of the HER3 antibodies disclosed in International Pub. Nos. WO 2019185164 and WO2019185878, U.S. Pat. No. 10,662,241; and U.S. Pub. Nos. 20190300624, 20210024651, and 20200308275.

Exemplary HER3 antibodies also include the HER2/HER3 bispecific antibody MCLA-128 (i.e., Zenocutuzumab) from Merus N.V.; and any of the HER3 antibodies, whether monospecific or multi-specific, disclosed in U.S. Pub. Nos. 20210206875, 20210155698, 20200102393, 20170058035, and 20170037145.

Exemplary HER3 antibodies also include the HER3 antibody Patritumab (U3-1287), an antibody including heavy chain sequence SEQ ID NO:106 and/or light chain sequence SEQ ID NO:7 which are reported chains of Patritumab, and any of the HER3 antibodies disclosed in U.S. Pat. Nos. 9,249,230 and 7,705,130 and International Pub. No. WO2007077028.

Exemplary HER3 antibodies also include the HER3 antibody MM-121 and any of the HER3 antibodies disclosed in U.S. Pat. No. 7,846,440 and International Pub. No. WO2008100624. Exemplary HER3 antibodies also include the EGFR/HER3 bispecific antibody DL1 and any of the HER3 antibodies, whether monospecific or multi-specific, disclosed in U.S. Pat. Nos. 9,327,035 and 8,597,652, U.S. Pub. No. 20140193414, and International Pub. No. WO2010108127.

Exemplary HER3 antibodies also include the HER2/HER3 bispecific antibody MM-111 and any of the HER3 antibodies, whether monospecific or multi-specific, disclosed in U.S. Pub. Nos. 20130183311 and 20090246206 and International Pub. Nos. WO2006091209 and WO2005117973.

According to certain aspects, the HER3 targeting agent includes an anti-HER3 antibody that binds to an epitope of HER3 recognized by Patritumab from Daiichi Sankyo, Seribantumab (MM-121) from Merrimack Pharmaceuticals, Lumretuzumab from Roche, Elgemtumab from Novartis, GSK2849330 from GlaxoSmithKline, CDX-3379 of Celldex Therapeutics, EV20 and MP-RM-1 from MediPharma, Barecetamab (ISU104) from Isu Abxis Co., HMBD-001 (10D1F) from Hummingbird Bioscience Pte., REGN1400 from Regeneron Pharmaceuticals, and/or AV-203 from AVEO Oncology. According to certain aspects, the anti-HER3 antibody is selected from one or more of Patritumab, Seribantumab or an antibody including heavy chain sequence SEQ ID NO:108 and/or light chain sequence SEQ ID NO:109 which are reported for Seribantumab, Lumretuzumab or an antibody including heavy chain sequence SEQ ID NO:110 and/or light chain sequence SEQ ID NO:111 which are reported for Lumretuzumab, Elgemtumab or an antibody including heavy chain sequence SEQ ID NO:112 and/or light chain sequence SEQ ID NO:113 which are reported for Elgemtumab, AV-203, CDX-3379, GSK2849330, EV20, MP-RM-1, ISU104, HMBD-001 (10D1F), and REGN1400. Exemplary antibodies along with exemplary treatment indications are also described in Table 1.

TABLE 1

| Company Name | | | Therapeutic | |
|---|---|---|---|---|
| (Originator) | Product Name | Targets | Modality | Exemplary Indications |
| Aveo Pharmaceuticals | CAN017, AV-203 | HER3 | Antibody | Esophageal cancer, solid |
| Inc. | | | | tumors |
| Celldex Therapeutics | CDX-3379, | HER3 | Antibody | Head and neck cancer, solid |
| Inc. | ktn3379 | | | tumors |
| Daiichi Sankyo Co. | patritumab | HER3 | Antibody | Non-small cell lung cancer |
| Ltd. | (AMG 888, U3- | | | (NSCLC), breast cancer, head |
| | 1287) | | | and neck cancer |
| Daiichi Sankyo Co. | U3-1402 | HER3 | Antibody- | NSCLC, breast cancer, colon |
| Ltd. | | | drug | cancer |
| | | | conjugate | |
| GSK | GSK2849330 | HER3 | Antibody | Solid tumors |
| Hummingbird | HMBD-001 | HER3 | Antibody | Gastric cancer |
| Bioscience Pte. Ltd. | (10D1F) | | | |
| Isu Abxis Co. Ltd. | ISU104 | HER3 | Antibody | Cancer (unspecified) |
| MediPharma | MP-RM-1 | HER3 | Antibody | Solid tumors |
| MediPharma | EV20 | HER3 | Antibody | Solid tumors |
| Merrimack | Seribantumab | HER3 | Antibody | NSCLC, breast cancer, |
| Pharmaceuticals Inc. | (MM-121, | | | ovarian cancer |
| | SAR256212) | | | |
| Novartis AG | elgemtumab | HER3 | Antibody | Esophageal cancer, Breast |
| | (LJM716) | | | cancer, solid tumors |
| Regeneron | REGN1400 | HER3 | Antibody | Cancer (unspecified) |
| Pharmaceuticals Inc. | | | | |
| Roche | Lumretuzumab | HER3 | Antibody | Breast cancer, solid tumors |
| | (RG7116 or | | | |
| | RO5479599) | | | |

It should be understood that wherever in this disclosure specific antibodies, specific antibody heavy chains and specific antibody light chains are disclosed, against HER3 or against any target, also intended to be disclosed for embodiment in or use in the various aspects of the invention are antibodies, such as but not limited to immunoglobulins, such as but not limited to IgG, that (i) include the heavy chain variable region of the disclosed antibody or heavy chain, (ii) include 1, 2 or 3 of the heavy chain CDRs (e.g., by Kabat definition) of the disclosed antibody or heavy chain, (iii) include the light chain variable region of the disclosed antibody or light chain, and/or (iv) include 1, 2 or 3 of the light chain CDRs (e.g., by Kabat definition) of the disclosed antibody or light chain. It should also be understood that wherever in this disclosure an antibody heavy chain or an antibody light chain is disclosed that includes an N-terminal leader sequence, also intended to be disclosed for embodiment in and use in the various aspects of the invention are corresponding heavy chains and corresponding light chains that lack the leader sequence.

Further, the invention provides modified versions of any of the recited amino acid sequences in which one or more isomeric amino acid replacements with exact mass, such as Leu for Ile or vice versa, are made (in, e.g., any of SEQ ID NOS:1-14 listed in FIGS. 1 and 2). Additionally, certain portions of these sequences may be substituted, such as by related portions from human immunoglobulins to form chimeric immunoglobulins (i.e., chimeric or humanized HER3). Exemplary substitutions include all or portions of the human leader sequence, and/or the conserved regions from human IgG1, IgG2, or IgG4 heavy chains and/or human Kappa light chain.

The sequence and structure of human HER3, human HER2, and human EGFR (HER1) are all known. An amino acid sequence of the human HER3 precursor protein (receptor tyrosine-protein kinase erbB-3 isoform 1 precursor NCBI Reference Sequence: NP_001973.2) is provided herein as SEQ ID NO:115. Those skilled in the art will readily appreciate that given known target protein amino acid sequences, various types of suitable antibodies and antibody mimetics specific for the extracellular domain of HER3, such as of human HER3, for use in the various aspects of the invention, may be produced using immunization and/or panning and/or antibody engineering techniques that are well established in the art.

A HER3 targeting agent that is radiolabeled for use in the various embodiments of the invention may, for example, include a HER3 binding peptide such as chelator-bearing HER3 binding peptide, such as a DOTA-bearing HER3 binding peptide, such as any of those disclosed in U.S. Pub. No. 20200121814.

According to certain aspects, the HER3 targeting agent includes/is a multi-specific targeting agent, such as a multi-specific antibody, against a first epitope of HER3 and at least a second epitope of HER3, or against HER3 and one or more different antigens such as one or more of EGFR (HER1), HER2, TROP2, and T-cell receptor gamma (TCRγ) chain alternate reading frame protein (TRAP). Exemplary multi-specific antibodies that may be used include bispecific antibodies against HER3/HER2 such as MM-111 from Merrimack Pharmaceuticals or MCLA-128 (i.e., Zenocutuzumab) from Merus N.V.; or against IGF-1R/HER3 such as MM-141 (i.e., Istiratumab) from Merrimack Pharmaceuticals; or against EGFR/IER3 such as MEHD7945A (i.e., Duligotumab) from Roche or any of the cetuximab-based bispecific or multi-specific zybodies from Zyngenia Inc.

According to certain aspects, a composition including a mixture of a HER3 targeting agent, such as an antibody against HER3, and one or more antibodies against one or more different antigens, in which one or more of the antibodies is radiolabeled, is provided and/or used. An exemplary antibody composition including an antibody mixture includes at least Sym013 from Symphogen having six monoclonal antibodies against EGFR (HER1), HER2, and HER3. In one aspect of the invention, one or more of the antibodies, such as an anti-HER3 antibody, of Sym013 may be radiolabeled. A related aspect of the invention provides a composition including targeting agents against EGFR (HER1), HER2, ad HER3, such as antibodies, in which one or more in any combination or all are radiolabeled

The present invention further provides multi-specific targeting agents, compositions and related methods of treating a proliferative disease or disorder which include administration of (i) a multi-specific antibody against two or more epitopes of HER3, or against an epitope of HER3 and an epitope of one or more additional different antigens, and/or (ii) administration of a HER3 targeting agent such as an antibody and one or more discrete targeting agents directed against one or more cancer associated antigens wherein one or more of the targeting agents, such as the HER3 targeting agent is radiolabeled. The additional different antigens may, for example, be antigens whose expression is upregulated on cells involved in various diseases or disorders, such as proliferative disorders, for example, solid tumor cancers, such as those in which HER3 is also or can also be upregulated. For example, the additional different antigens may be selected from the group including mesothelin, TSHR, CD19, CD123, CD22, CD30, CD45, CD171, CD138, CS-1, CLL-1, GD2, GD3, B-cell maturation antigen (BCMA), Tn Ag, prostate specific membrane antigen (PSMA), ROR1, FLT3, TROP2, T-cell receptor gamma (TCRγ) chain alternate reading frame protein (TRAP), fibroblast activation protein (FAP), calreticulin, phosphatidylserine, GRP78 (BiP), TAG72, CD38, CD44v6, CEA, EPCAM, B7H3, KIT, IL-13Ra2, interleukin-11 receptor a (IL-11Ra), PSCA, PRSS21, VEGFR2, LewisY, CD24, platelet-derived growth factor receptor-beta (PDGFR-beta), SSEA-4, CD20, Folate receptor alpha (FRa), ERBB2 (Her2/neu), MUCI, epidermal growth factor receptor (EGFR), EGFRvIII, NCAM, Prostase, PAP, ELF2M, Ephrin B2, IGF-I receptor, CAIX, LMP2, gplOO, bcr-abl, tyrosinase, EphA2, Fucosyl GM1, sLe, GM3, DR5, 5T4, TGS5, HMWMAA, o-acetyl-GD2, Folate receptor beta, TEM1/CD248, TEM7R, CLDN6, GPRC5D, CXORF61, CD97, CD 179a, ALK, Polysialic acid, PLAC1, GloboH, NY-BR-1, UPK2, HAVCR1, ADRB3, PANX3, GPR20, LY6K, OR51E2, TARP, WT1, NY-ESO-1, LAGE-1a, MAGE-A1, legumain, HPV E6,E7, MAGE Al, MAGEA3, MAGEA3/A6, ETV6-AML, sperm protein 17, XAGE1, Tie 2, MAD-CT-1, MAD-CT-2, Fos-related antigen 1, prostein, survivin and telomerase, PCTA-1/Galectin 8, KRAS, MelanA/MARTI, Ras mutant, hTERT, sarcoma translocation breakpoints, ML-IAP, ERG (TMPRSS2 ETS fusion gene), NA17, PAX3, Androgen receptor, Cyclin B 1, MYCN, RhoC, TRP-2, CYP1B 1, BORIS, SART3, PAX5, OY-TES 1, LCK, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, GPA7, and IGLL1.

Exemplary DR5 (death receptor 5) targeting agents that may be radiolabeled, unlabeled or drug-conjugated for use in the invention include the monoclonal anti-DR5 antibodies mapatumumab, conatumumab, lexatumumab, tigatuzumab, drozitumab, and LBY-135. Such DR5 targeting agents may, for example, be used in combination with a radiolabeled HER3 targeting agents for the treatment of ovarian, breast, cervical prostate, gastric, bladder, lung, melanoma, colorectal and squamous cell carcinoma cancers and any of the cancers disclosed herein.

Exemplary 5T4 (Trophoblast glycoprotein (TBPG)) targeting agents that may be radiolabeled, drug-conjugated, or unlabeled for use in the invention include the anti-5T4 monoclonal antibodies MED10641, ALG.APV-527, Tb535, H6-DM5, and ZV0508, as well as Naptumomab estafenatox or the Fab portion thereof. Such 5T4 targeting agents may, for example, be used in combination with a radiolabeled HER3 targeting agent for the treatment of ovarian, head and neck, breast, prostate, gastric, bladder, lung, melanoma, colorectal and squamous cell carcinoma cancers and any of the cancers disclosed herein.

Exemplary HER2 (ERBB2) targeting agents that may be radiolabeled, drug-conjugated, or unlabeled for use in the invention include the monoclonal antibodies trastuzumab and pertuzumab. Applicants have successfully conjugated Trastuzumab with p-SCN-DOTA and radiolabeled the composition with ²²⁵Ac or ¹⁷⁷Lu. Exemplary ADCs targeting HER2 that may be used include fam-trastuzumab deruxtecan-nxki (Enhertu®; AstraZeneca/Daiichi Sankyo) and Trastuzumab emtansine (Roche/Genentech). The anti-HER2 antibody may, for example, also be a multi-specific antibody, such as bispecific antibody, against any available epitope of HER3/IER2 such as MM-111 and MM-141/Istiratumab from Merrimack Pharmaceuticals, MCLA-128 from Merus NV, and MEHD7945A/Duligotumab from Genentech. HER2 targeting agents may, for example, be used in combination with a radiolabeled HER3 targeting agent in the treatment of HER2-expressing cancers such as ovarian, breast, metastatic breast, esophageal, lung, cervical, and endometrial cancers including but not limited to those that are both HER2- and HER3-positive.

The amino acid sequences of the heavy chain and the light chain of Trastuzumab reported by DrugBank Online are: heavy chain (SEQ ID NO:102) and light chain (SEQ ID NO:103) and a HER2 binding antibody including one or both of said chains may be embodied in or used in the various embodiments of the invention.

The amino acid sequences of the heavy chain and the light chain of Pertuzumab reported by DrugBank Online are: heavy chain (SEQ ID NO:104) and light chain (SEQ ID NO:105) and a HER2 binding antibody including one or both of said chains may be embodied in or used in the various embodiments of the invention.

Exemplary CD33 targeting agents that may be radiolabeled, drug-conjugated, or unlabeled for use in the invention include the monoclonal antibodies lintuzumab, gemtuzumab, and vadastuximab. In combination with a radiolabeled HER3 targeting agent as disclosed herein, a CD33 targeting therapeutic agent may, for example, be used to treat solid cancers, such as ovarian, breast, cervical prostate, gastric, bladder, lung, melanoma, colorectal and squamous cell carcinoma cancers and any of the cancers disclosed herein, for example, by depleting myeloid-derived suppressor cells (MDSCs). In one aspect, the CD33 targeting agent used in combination with a radiolabeled HER3 targeting agent is 225Ac-lintuzumab. In another aspect, the CD33 targeting agent used in combination with a radiolabeled HER3 targeting agent is the ADC gemtuzumab ozogamicin (Mylotarg®; Pfizer).

Exemplary CD38 targeting agents that may be radiolabeled, drug-conjugated, or unlabeled for use in the invention include anti-CD38 monoclonal antibodies such as daratumumab (Darzalex®; Johnson and Johnson) and isatuximab (Sarclisa®; Sanofi) or antigen-binding fragments thereof. Such CD38 targeting agents may, for example, be used in combination with the radiolabeled HER3 targeting agents in the treatment of solid tumors that may, for example, be infiltrated with CD38-positive suppressive immune cells, such as but not limited to ovarian, breast, cervical prostate, gastric, bladder, lung, melanoma, colorectal and squamous cell carcinoma cancers and any of the cancers disclosed herein.

Exemplary different antigens (over HER3) that may be targeted by a multi-specific antibody according to aspects of the present invention include at least HER1 (EGFR), HER2, and IGF-1R. Exemplary HER3 multi-specific targeting agents include multi-specific antibodies such as MM-111 from Merrimack Pharmaceuticals or MCLA-128 (i.e., Zenocutuzumab) from Merus N.V.; or against IGF-1R/HER3 such as MM-141 (i.e., Istiratumab) from Merrimack Pharmaceuticals; or against EGFR/IER3 such as MEHD7945A (i.e., Duligotumab) from Roche, the cetuximab-based bispecific zybody from Zyngenia Inc., and the multi-specific antibody composition Sym-013 from Symphogen. See also Table 2 for further description and exemplary indications.

TABLE 2

| Company Name | | | Therapeutic | Exemplary |
|---|---|---|---|---|
| (Originator) | Product Name | Targets | Modality | Indications |
| Merrimack | Istiratumab | IGF-1R; HER3 | Bispecific Antibody | Solid tumors |
| Pharmaceuticals | (MM-141) | | | |
| Inc. | | | | |
| Merrimack | MM-111 | HER2; HER3 | Bispecific Antibody | Breast cancer, solid |
| Pharmaceuticals | | | | tumors |
| Inc. | | | | |
| Merus N.V. | MCLA-128 | HER2; HER3 | Bispecific Antibody | NSCLC, breast |
| | | | | cancer, ovarian |
| | | | | cancer, colorectal |
| | | | | cancer, gastric |
| | | | | cancer, endometrial |
| | | | | cancer, solid tumors |
| Roche | Duligotuzmab | EGFR; HER3 | Antibody | Colorectal cancer, |
| | (MEHD7945A, | | | epithelial cancer, |
| | RG7597) | | | head and neck |
| | | | | cancer, solid tumors |
| Symphogen | Sym013 | HER1, HER2, | Antibody (mixture) | Solid tumors |
| | | HER3 | | |
| Zyngenia Inc. | Cetuximab-based | EGFR; HER3 | Antibody | Cancer (unspecified) |
| | bispecific zybody | | | |

The present invention also provides methods of treating a proliferative disease or disorder that includes administration of a first antibody against at least one epitope of HER3, and administration of a second antibody, wherein the second antibody is against a different epitope of HER3 than the first antibody, or is against an epitope of a different antigen, such as one or more antigens selected from the list of different antigens presented above. One or more of the HER3 antibodies may be radiolabeled. Antibodies against the different antigens may, for example, also be radiolabeled in any combination.

Such combinations, presented as a multi-specific antibody or more than one monoclonal antibody as indicated above, may deliver a synergistic therapeutic effect comparable to the effectiveness of a monotherapy with only an antibody against HER3, while reducing adverse side effects of the monotherapy. Moreover, the combination may deliver an improved effectiveness over the monotherapy, which may, for example, be measured by reduction in the total tumor cell number, increase in the length of time to relapse, and other indicia of patient health.

When the methods include administration of a multi-specific antibody, the first target recognition component may, for example, include one of: a first full-length heavy chain and a first full-length light chain, a first Fab fragment, a first single-chain variable fragment (scFvs), or other type of antibody. The second target recognition component may, for example, include one of: a second full length heavy chain and a second full length light chain, a second Fab fragment, or a second single-chain variable fragment (scFvs) or other type of antibody. Moreover, the second target recognition component may be derived from a different epitope of the HER3 antigen or may be derived from any of the antigens listed above.

A HER3 targeting agent may include a radioisotope, and any additional antibodies against other antigens may optionally include a radioisotope. According to certain aspects of the present invention, when the immunotherapy includes a bispecific antibody, either one or both of the first target recognition component and the second target recognition component, or any part of the bispecific targeting agent, may include a radioisotope.

According to certain aspects of the present invention, the radiolabeled targeting agent may exhibit essentially the same immunoreactivity to the antigen as a control targeting agent, wherein the control targeting agent includes the naked targeting agent or otherwise unlabeled targeting agent against the same epitope of the antigen (i.e., HER3) as the radiolabeled targeting agent.

According to certain aspects of the present invention, the targeting agent may be labeled with ²²⁵Ac, and may be at least 5-fold more effective at causing cell death of HER3-positive cells than a control monoclonal antibody, wherein the control monoclonal antibody includes a naked or unlabeled antibody against the same epitope of the antigen as the ²²⁵Ac labeled antibody. For example, a ²²⁵Ac labeled monoclonal antibody may be at least 10-fold more effective, at least 20-fold more effective, at least 50-fold more effective, or at least 100-fold more effective at causing cell death of HER3-positive cells than the control monoclonal antibody.

According to certain aspects of the present invention, the methods may include administration of labeled and unlabeled (e.g., “naked”) fractions of the HER3 targeting agent, such as an antibody, antibody fragment, etc. For example, the un-labeled fraction may include the same antibody against the same epitope as the labeled fraction. In this way, the total radioactivity of the antibody may be varied or may be held constant while the overall antibody protein concentration may be held constant or may be varied, respectively. For example, the total protein concentration of un-labeled antibody fraction administered may vary depending on the exact nature of the disease to be treated, age and weight of the patient, identity of the monoclonal antibody, and the label (e.g., radionuclide) selected for labeling of the monoclonal antibody.

According to certain aspects of the present invention, the effective amount of the anti-HER3 antibody is a maximum tolerated dose (MTD) of the anti-HER3 antibody.

According to certain method aspects of the present invention, when more than one antibody is administered, the antibodies may be administered at the same time. As such, according to certain aspects of the present invention, the antibodies may be provided in a single composition. Alternatively, the two antibodies may be administered sequentially. As such, the radiolabeled HER3 targeting agent may be administered before the second antibody, after the second antibody, or both before and after the second antibody. Moreover, the second antibody may be administered before the radiolabeled HER3 targeting agent, after the radiolabeled HER3 targeting agent, or both before and after the radiolabeled HER3 targeting agent.

According to certain aspects of the methods of the present invention, a radiolabeled HER3 targeting agent may be administered according to a dosing schedule selected from the group consisting of one every 7, 10, 12, 14, 20, 24, 28, 35, and 42 days throughout a treatment period, wherein the treatment period includes at least two doses.

According to certain aspects of the present invention, the radiolabeled HER3 targeting agent may be administered according to a dose schedule that includes 2 doses, such as on days 1 and 5, 6, 7, 8, 9, or 10 of a treatment period, or days 1 and 8 of a treatment period.

Administration of the radiolabeled HER3 targeting agents of the present invention, in addition to other therapeutic agents, may be provided in a number of ways depending upon whether local or systemic treatment is desired and upon the area to be treated. Administration may be intratracheal, intranasal, epidermal and transdermal, oral or parenteral. Parenteral administration includes intravenous, intra-arterial, subcutaneous, intraperitoneal or intramuscular injection or infusion; or intracranial, e.g., intrathecal or intraventricular, administration. In some embodiments a slow-release preparation including the targeting agents(s) and/or other therapeutic agents may be administered. The various agents may be administered as a single treatment or in a series of treatments that continue as needed and for a duration of time that causes one or more symptoms of the cancer to be reduced or ameliorated, or that achieves another desired effect.

The dose(s) may vary, for example, depending upon the identity, size, and condition of the subject, further depending upon the route by which the composition is to be administered and the desired effect. Appropriate doses of a therapeutic agent depend upon the potency with respect to the expression or activity to be modulated. The therapeutic agents can be administered to an animal (e.g., a human) at a relatively low dose at first, with the dose subsequently increased until an appropriate response is obtained.

The radiolabeled HER3 targeting agent may be administered simultaneously or sequentially with the one or more additional therapeutic agents. Moreover, when more than one additional therapeutic agent is included, the additional therapeutic agents may be administered simultaneously or sequentially with each other and/or with the radiolabeled HER3 targeting agent.

Radiolabeling the HER3 Targeting Agent

The HER3 targeting agent and other targeting agents disclosed herein may, for example, be labeled with a radioisotope, such as a beta emitter (e.g. ¹⁷⁷Lu) or an alpha emitter (e.g., ²²⁵Ac), through conjugation of a chelator molecule, and chelation of the radioisotope thereto. According to certain aspects, the targeting agent may be an antibody against that is deglycosylated in the constant region, such as at asparagine-297 (Asn-297, N297; Kabat number) in the heavy chain CH2 domain, for the purpose of uncovering a unique conjugation site, glutamine (i.e., Gln-295, Q295) so that it is available for conjugation with bifunctional chelator molecules.

According to certain aspects, the radiotherapeutic may be an antibody that may have reduced disulfide bonds such as by using reducing agents, which may then be converted to dehydroalanine for the purpose of conjugating with a bifunctional chelator molecule.

According to certain aspects, the radiotherapeutic may be an antibody for which the disulfide bonds have been reduced using reducing agents, which is then conjugated via aryl bridges with a bifunctional chelator molecule. For example, according to certain aspects a linker molecule such as 3,5-bis(bromomethyl)benzene may be used to bridge the free sulfhydryl groups on the antibody.

According to certain aspects, the radiotherapeutic may be an antibody that may have certain specific existing amino acids replaced with cysteine(s) that then can be used for site-specific labeling.

Exemplary chelators that may be linked to targeting agents in the various aspects of the invention include: 1,4,7,10-tetraazacyclododecane-1,4,7-triacetic acid (DO3A) or a derivative thereof; 1,4,7-triazacyclononane-1,4-diacetic acid (NODA) or a derivative thereof, 1,4,7-triazacyclononane-1,4,7-triacetic acid (NOTA) or a derivative thereof; 1,4,7,10-tetraazacyclododecane-1,4,7,10-tetraacetic acid (DOTA) or a derivative thereof; 1,4,7-triazacyclononane, 1-glutaric acid-4,7-diacetic acid (NODAGA) or a derivative thereof, 1,4,7,10-tetraazacyclodecane, 1-glutaric acid-4,7,10-triacetic acid (DOTAGA) or a derivative thereof; 1,4,8,11-tetraazacyclotetradecane-1,4,8,11-tetraacetic acid (TETA) or a derivative thereof, 1,4,8,11-tetraazabicyclo[6.6.2]hexadecane-4,11-diacetic acid (CB-TE2A) or a derivative thereof, diethylene triamine pentaacetic acid (DTPA), its diester, or a derivative thereof; 2-cyclohexyl diethylene triamine pentaacetic acid (CHX-A″-DTPA) or a derivative thereof; deforoxamine (DFO) or a derivative thereof, 1,2-[[6-carboxypyridin-2-yl]methylamino]ethane (H₂dedpa) or a derivative thereof, DADA or a derivative thereof; 1,4,7,10-Tetraazacyclododecane-1,4,7,10-tetra(methylene phosphonic acid) (DOTP) or a derivative thereof, 4-amino-6-[[16-[(6-carboxypyridin-2-yl)methyl]-1,4,10,13-tetraoxa-7,16-diazacyclooctadec-7-yl]methyl]pyridine-2-carboxylic acid (MACROPA-NH₂) or a derivative thereof, MACROPA or a derivative thereof, 1,4,7,10-tetrakis(carbamoylmethyl)-1,4,7,10-tetraazacyclododecane (TCMC) or a derivative thereof; {4-[2-(bis-carboxymethylamino)-ethyl]-7-carboxymethyl-[1,4,7]triazonan-1-yl}-acetic acid (NETA) or a derivative thereof; Diamsar or a derivative thereof; 1,4,7-triazacyclononane-1,4,7-tris[methyl(2-carboxyethyl)phosphinic acid (TRAP, PRP9, TRAP-Pr) or a derivative thereof; N,N′-bis(6-carboxy-2-pyridylmethyl)ethylenediamine-N,N′-diacetic acid (H4octapa) or a derivative thereof, N,N′-[1-benzyl-1,2,3-triazole-4-yl]methyl-N,N′-[6-(carboxy)pyridin-2-yl]-1,2-diaminoethane (H2azapa) or a derivative thereof, N,N″-[[6-(carboxy)pyridin-2-yl]methyl]diethylenetriamine-N,N′,N″-triacetic acid (H5decapa) or a derivative thereof; N,N′-bis(2-hydroxy-5-sulfobenzyl)ethylenediamine-N,N′-diacetic acid (SHBED) or a derivative thereof; N,N′-bis(2-hydroxybenzyl)ethylenediamine-N,N′-diacetic acid (HBED) or a derivative thereof; 3,6,9,15-tetraazabicyclo[9.3.1]pentadeca-1(15),11,13-triene-3,6,9,-triacetic acid (PCTA) or a derivative thereof, desferrioxamine B (DFO) or a derivative thereof; N,N′-(methylenephosphonate)-N,N′-[6-(methoxycarbonyl)pyridin-2-yl]methyl-1,2-diaminoethane (H6phospa) or a derivative thereof, 1,4,7,10,13,16-hexaazacyclohexadecane-N,N′,N″,N′″,N″″,N′″″-hexaacetic acid (HEHA) or a derivative thereof, 1,4,7,10,13-pentaazacyclopentadecane-N,N′,N″,N′″,N″″-pentaacetic acid (PEPA) or a derivative thereof, or 3,4,3-LI(1,2-HOPO) or a derivative thereof.

According to certain aspects, the targeting agent may be radiolabeled through chemical conjugation of suitable bifunctional chelators that can chelate one or more radionuclides. Exemplary chelator molecules that may be used include p-SCN-Bn-DOTA, NH₂-DOTA, NH₂—(CH₂)₁₋₂₀-DOTA, NH₂—(PEG)₁₋₂₀-DOTA, HS-DOTA, HS—(CH₂)₁₋₂₀-DOTA, HS—(PEG)₁₋₂₀-DOTA, dibromo-S—(CH₂)₁₋₂₀-DOTA, dibromo-S—(PEG)₁₋₂₀-DOTA, p-SCN-Bn-DOTP, NH₂-DOTP, NH₂—(CH₂)₁₋₂₀-DOTP, NH₂—(PEG)₁₋₂₀-DOTP, HS-DOTP, HS—(CH₂)₁₋₂₀-DOTP, HS—(PEG)₁₋₂₀-DOTP, dibromo-S—(CH₂)₁₋₂₀-DOTP, and dibromo-S—(PEG)₁₋₂₀-DOTP.

The chelator molecules may, for example, be attached to a targeting agent through a linker molecule. Exemplary linker molecules include:

—CH₂(C₆H₄)NH₂ or —CH₂(C₆H₄)NH—X—Y,
wherein X is
—R₂—CH₂CH₂O(CH₂CH₂O)ₙCH₂CH₂—,
—R₂—CH₂CH₂NHC(O)CH₂CH₂O(CH₂CH₂O)ₙCH₂CH₂—,
—R₂—(CH₂)ₙCH₂—,
—R₂—CH₂CH₂NHC(O)(CH₂)ₙCH₂—,
—R₂—CH(C(O)R₃)CH₂—, wherein R₃ is —OH or a short peptide (1-20 amino acids),
—R₂—CH₂CH₂O(CH₂CH₂O)ₙCH₂C(O)O—, or
—R₂—CH₂CH₂NHC(O)CH₂CH₂O(CH₂CH₂O)ₙCH₂CC(O)O—,
wherein n is 1-20, and

### R₂ is —C(O)— or —C(S)NH—; and

Y is —NH₂ or —SR₄—, wherein R₄ is —H or —CH₂-3,5-bis(bromomethyl)benzene.

Targeting agents, such as protein targeting agents, for example antibodies and antigen-binding antibody fragments, and peptide targeting agents may, for example, be conjugated with a chelator for radiolabeling the targeting agent via chelation of a radionuclide. Such protein or peptide targeting agents, for example, that include lysine(s), may conveniently be conjugated to a DOTA chelating moiety using the bifunctional agent S-2-(4-Isothiocyanatobenzyl)-1,4,7,10-tetraazacyclododecane tetraacetic acid a/k/a/ “p-SCN-Bn-DOTA” (Catalog #B205; Macrocyclics, Inc., Plano, Tex., USA). p-SCN-Bn-DOTA may be synthesized by a multi-step organic synthesis fully described in U.S. Pat. No. 4,923,985. Chelation of a radionuclide by the DOTA moiety may be performed prior to chemical conjugation of the antibody with p-SCN-Bn-DOTA and/or after said conjugation.

Methods for labeling a chelator-conjugated targeting agent with an exemplary radionuclide are described in in Example 1.

Diagnostic Aspects

The presently disclosed methods may include diagnosing the subject to ascertain if HER3-positive cells are present, to what extent they are, and/or their localization. HER3-positive cells may be present in a number of biological specimens, such as in circulating cells in a sample of blood from the subject or tumor cells in a biopsy of the subject. In one aspect, the diagnosing step may generally include obtaining a sample of blood or tissue from the subject and mounting the sample on a substrate. The presence or absence of the HER3 antigen may be detected using a diagnostic antibody, peptide, or small molecule, wherein the diagnostic antibody peptide, or small molecule is labeled with any of the standard imaging labels known in the art. Exemplary labeling agents include, for example, radiolabels such as ³H, ¹⁴C, ³²P ³⁵S, and ¹²⁵I; fluorescent or chemiluminescent compounds, such as fluorescein isothiocyanate, rhodamine, or luciferin; and enzymes, such as alkaline phosphatase, β-galactosidase, or horseradish peroxidase. An exemplary HER3 targeting agent used in such a diagnostic assay includes a human or humanized antibody against HER3.

In another aspect, the methods may include diagnosing the subject to ascertain if HER3-positive cells are present using a HER3 targeting agent labeled with a radionuclide such as any of ¹⁸F, ¹¹C, ⁶⁸Ga, ⁶⁴Cu, ⁸⁹Zr, or ¹²⁴I, for PET imaging, or ^{99m}Tc or ¹¹¹In, for SPECT imaging. Accordingly, the method may include administering to the subject a HER3 targeting agent labeled with one or more of ¹⁸F, ¹¹C, ⁶⁸Ga, 64Cu, ⁸⁹Zr, ¹²⁴I, ^{99m}Tc, or ¹¹¹In, and performing a non-invasive imaging technique on the subject, such as performing a PET or SPECT scan on the subject. The method may include administering the radiolabeled HER3 targeting agent for imaging to the subject and, after an amount of time sufficient for the targeting agent to bind to target in the subject's tissues, performing the imaging. The amount of time sufficient for the targeting agent to bind to target in the subject's tissues may, for example, be at least 20 minutes, at least 30 minutes, at least 60 minutes, or any number or subrange of minutes in the range 20 minutes to 360 minutes. According to certain one aspect of the method, the radiolabeled HER3 targeting agent may include ⁶⁸Ga, ⁸⁹Zr, or ¹¹¹In, and may be labeled using any of the methods disclosed herein (e.g., such as disclosed in Example 1).

If the subject has HER3-positive cancer cells, for example, beyond a predetermined or preselected threshold level, or other indications of a HER3-positive cancer/tumor, the therapeutic methods of the presently disclosed invention may be carried out, i.e., administration of a therapeutically effective amount of a radiolabeled HER3 targeting agent, alone or in combination with one or more additional therapeutic agents may be performed.

Additional Therapeutic Agents and Modalities

The methods of the present invention that include administration of a radiolabeled HER3 targeting agent therapeutic, alone or in combination with other targeting agents, may further include administration of an additional therapeutic agent or modality. According to certain aspects, the additional agent may be relevant for the disease or condition being treated by the radiolabeled HER3 targeting agent. Such administration may be simultaneous, separate or sequential with the administration of the effective amount of the HER3 targeting agent. For simultaneous administration, the agents may be administered as one composition, or as separate compositions, as appropriate.

Exemplary additional therapeutic agents and modalities that may be used in combination or conjunction with a radiolabeled HER3 targeting agent include at least chemotherapeutic agents, small molecule oncology drugs, anti-inflammatory agents, immunosuppressive agents, immunomodulatory agents, include immune checkpoint therapies, DDR inhibitors, CD47 blockades, external beam radiation, brachytherapy, or any combination thereof. Exemplary additional agents and treatment modalities that may be used in combination or conjunction with a radiolabeled HER3 targeting agent alone or in combination other targeting agents as disclosed herein are further described below.

A. Chemotherapeutic and Other Small Molecule Agents

Exemplary chemotherapeutic agents include, but are not limited to, anti-neoplastic agents including alkylating agents including: nitrogen mustards, such as mechlorethamine, cyclophosphamide, ifosfamide, melphalan and chlorambucil; nitrosoureas, such as carmustine (BCNU), lomustine (CCNU), and semustine (methyl-CCNU); Temodal™ (temozolomide), ethylenimines/methylmelamine such as thriethylenemelamine (TEM), triethylene, thiophosphoramide (thiotepa), hexamethylmelamine (HMM, altretamine); alkyl sulfonates such as busulfan; triazines such as dacarbazine (DTIC); antimetabolites including folic acid analogs such as methotrexate and trimetrexate, pyrimidine analogs such as 5-fluorouracil (5FU), fluorodeoxyuridine, gemcitabine, capecitabine (e.g. Xeloda®), cytosine arabinoside (AraC, cytarabine), 5-azacytidine, 2,2′-difluorodeoxycytidine, purine analogs such as 6-mercaptopurine, 6-thioguamne, azathioprine, T-deoxycoformycin (pentostatin), erythrohydroxynonyladenine (EHNA), fludarabine phosphate, and 2-chlorodeoxyadenosine (cladribine, 2-CdA); natural products including antimitotic drugs such as paclitaxel, vinca alkaloids including vinblastine (VLB), vincristine, and vinorelbine, taxotere, estramustine, and estramustine phosphate; pipodophylotoxins such as etoposide and teniposide; antibiotics such as actinomycin D, daunomycin (rubidomycin), doxorubicin, mitoxantrone, idarubicin, bleomycins, plicamycin (mithramycin), mitomycin C, and actinomycin; enzymes such as L-asparaginase; biological response modifiers such as interferon-alpha, IL-2, G-CSF and GM-CSF; miscellaneous agents including platinum coordination complexes such as oxaliplatin, cisplatin and carboplatin, anthracenediones such as mitoxantrone, substituted urea such as hydroxyurea, methylhydrazine derivatives including N-methylhydrazine (MIH) and procarbazine, adrenocortical suppressants such as mitotane (o, p-DDD) and aminoglutethimide; hormones and antagonists including adrenocorticosteroid antagonists such as prednisone and equivalents, dexamethasone and aminoglutethimide; Gemzar™ (gemcitabine), progestin such as hydroxyprogesterone caproate, medroxyprogesterone acetate and megestrol acetate; estrogen such as diethylstilbestrol and ethinyl estradiol equivalents; antiestrogen such as tamoxifen; androgens including testosterone propionate and fluoxymesterone/equivalents; antiandrogens such as flutamide, gonadotropin-releasing hormone analogs and leuprolide; non-steroidal antiandrogens such as flutamide and enzalutamide (e.g., Xtandi®), and FOLFOX-6 regimen (leucovorin calcium (folinic acid), fluorouracil, and oxaliplatin).

Therapies targeting epigenetic mechanisms including, but not limited to: (i) histone deacetylase (HDAC) inhibitors such as Vorinostat (suberoylanilide hydroxamic acid; SAHA), Romidepsin, Belinostat (PDX101), Panobinostat (LBH589) and Tucidinostat, demethylating agents (e.g., Vidaza); (ii) LSD1 inhibitors such as seclidemstat, TCP (tranylcypromine), ORY-1001 (iadademstat), GSK2879552 (GSK), INCB059872 (Imago BioSciences), IMG-7289 (Bomedemstat; Imago BioSciences), ORY-2001 (Vafidemstat), and CC-90011 (Celgene); and (iii) release of transcriptional repression (ATRA) therapies, may also be used in combination or conjunction with a radiolabeled HER3 targeting agent and/or other radiolabeled targeting agents and combinations thereof as disclosed herein.

According to certain aspects of the present invention, the chemotherapeutic agents include at least radiosensitizers, such as temozolomide, cisplatin, and/or fluorouracil.

The additional agents may, for example, include a bcl-2 inhibitor such as navitoclax or venetoclax (Venclexta®; Abbvie) and the combination may, for example, be used for the treatment of solid tumors such as breast cancers and lunger cancer such as small cell lung carcinoma (SCLC).

The additional agents may, for example, include a cyclin-dependent kinase CDK4 and CDK6 inhibitor such as palbociclib (Ibrance®; Pfizer) and the combination may, for example, be used for the treatment of solid cancers such as breast cancers such as HR-positive and HER2-negative breast cancer, with or without an aromatase inhibitor.

The additional agents may, for example, include erlotinib (Tarceva®; Roche) and the combination may, for example, be used for the treatment of solid tumor cancers such as non-small cell lung cancer (NSCLC), for example, with mutations in the epidermal growth factor receptor (EGFR) and pancreatic cancer.

The additional agents may, for example, include sirolimus, everolimus (Affinitor®; Novartis), or temsirolimus and the combination may, for example, be used for the treatment of solid tumor cancers such as melanoma and breast cancer.

The additional agents may, for example, include pemetrexed (Alimta®; Eli Lilly) and the combination may, for example, be used for the treatment of solid cancers such as mesothelioma such as pleural mesothelioma and lung cancer such as non-small cell lung cancer (NSCLC).

The additional therapeutic agents may, for example, be administered according to any standard dose regime known in the field. For example, therapeutic agents may be administered at concentrations in the range of 1 to 500 mg/m², the amounts being calculated as a function of patient surface area (m²). For example, exemplary doses of the chemotherapeutic paclitaxel may include 15 mg/m² to 275 mg/m², exemplary doses of docetaxel may include 60 mg/m² to 100 mg/m², exemplary doses of epithilone may include 10 mg/m² to 20 mg/m², and an exemplary dose of calicheamicin may include 1 mg/m² to 10 mg/m². While exemplary doses are listed herein, such are only provided for reference and are not intended to limit the dose ranges of the drug agents of the presently disclosed invention.

B. External Beam Radiation and/or Brachytherapy

The additional therapeutic modality administered in conjunction with the HER3 targeting agent, and optionally any other of the additional therapeutics disclosed herein, may be an ionizing radiation, such as administered via external beam radiation or brachytherapy. Such radiation generally refers to the use of X-rays, gamma rays, or charged particles (e.g., protons or electrons) to generate ionizing radiation, such as delivered by a machine placed outside the patient's body (external-beam radiation therapy) or by a source placed inside a patient's body (internal radiation therapy or brachytherapy).

The external beam radiation or brachytherapy may enhance the targeted radiation damage delivered by the radiolabeled HER3 targeting agent and may thus be delivered sequentially with the HER3 targeting agent, such as before and/or after the HER3 targeting agent, or simultaneous with the HER3 targeting agents.

The external beam radiation or brachytherapy may be planned and administered in conjunction with imaging-based techniques such as computed tomography (CT) and/or magnetic resonance imaging (MRI) to accurately determine the dose and location of radiation to be administered. For example, a patient treated with any of the radiolabeled HER3 targeting agents disclosed herein may be imaged using either of CT or MRI to determine the dose and location of radiation to be administered by the external beam radiation or brachytherapy.

In various embodiments, the radiation therapy may be selected from the group consisting of total all-body radiation therapy, conventional external beam radiation therapy, stereotactic radiosurgery, stereotactic body radiation therapy, 3-D conformal radiation therapy, intensity-modulated radiation therapy, image-guided radiation therapy, tomotherapy, and brachytherapy. According to certain aspects, the radiation therapy may be provided as a single dose or as fractionated doses, e.g., as 2 or more fractions. For example, the dose may be administered such that each fraction includes 2-20 Gy (e.g., a radiation dose of 50 Gy may be split up into 10 fractions, each including 5 Gy). The 2 or more fractions may be administered on consecutive or sequential days, such as once in 2 days, once in 3 days, once in 4 days, once in 5 days, once in 6 days, once in 7 days, or in a combination thereof.

C. Immune Checkpoint Therapies

The additional agent(s) administered in conjunction with the HER3 targeting agent may be an immune checkpoint therapy. Cancer cells have developed means to evade the standard checkpoints of the immune system. For example, cancer cells have been found to evade immunosurveillance through reduced expression of tumor antigens, downregulation of MHC class I and II molecules leading to reduced tumor antigen presentation, secretion of immunosuppressive cytokines such as TGFb, recruitment or induction of immunosuppressive cells such as regulatory T cells (Treg) or myeloid-derived suppressor cells (MDSC), and overexpression of certain ligands [e.g., programmed death ligand-1 (PD-L1)] that inhibit the host's existing antitumor immunity.

Another major mechanism of immune suppression by cancer cells is a process known as “T-cell exhaustion”, which results from chronic exposure to tumor antigens, and is characterized by the upregulation of inhibitory receptors. These inhibitory receptors serve as immune checkpoints in order to prevent uncontrolled immune reactions.

Various immune checkpoints acting at different levels of T cell immunity have been described in the literature, including PD-1 (i.e., programmed cell death protein 1) and its ligands PD-L1 and PD-L2, CTLA-4 (i.e., cytotoxic T-lymphocyte associated protein-4) and its ligands CD80 and CD86, LAG3 (i.e., Lymphocyte-activation gene 3), B and T lymphocyte attenuator, TIGIT (T-cell immunoreceptor with Ig and ITIM domains), TIM-3 (i.e., T-cell immunoglobulin and mucin-domain containing protein 3), and VISTA (V-domain immunoglobulin suppressor of T cell activation).

Enhancing the efficacy of the immune system by therapeutic intervention is a particularly exciting development in cancer treatment. As indicated, checkpoint inhibitors such as CTLA-4 and PD-1 prevent autoimmunity and generally protect tissues from immune collateral damage. In addition, stimulatory checkpoints, such as OX40 (i.e., tumor necrosis factor receptor superfamily, member 4; TNFR-SF4), CD137 (i.e., TNFR-SF9), GITR (i.e., Glucocorticoid-Induced TNFR), CD27 (i.e., TNFR-SF7), CD40 (i.e., cluster of differentiation 40), and CD28, activate and/or promote the expansion of T-cells. Regulation of the immune system by inhibition or overexpression of these proteins is an area of promising current research.

Thus, a promising therapeutic strategy is the use of immune checkpoint therapies that may remove certain blockades on the immune system that are utilized by cancer cells, in combination with the HER3 targeting agents disclosed herein. For example, antibodies against certain immune checkpoint inhibitors (ICI) may block interaction between checkpoint inhibitor proteins and their ligands, therefore preventing the signaling events that would otherwise have led to inhibition of an immune response against the tumor cell.

Moreover, there is a growing body of preclinical evidence supporting the ability of radiation to synergize with ICI antibodies, and this is also being explored in the clinic with increasing numbers of clinical trials evaluating the combination of external beam radiation with immune checkpoint therapies across various tumor types and ICI antibodies (Lamichhane, 2018). Clinical evidence supporting this combination has been generated in melanoma, with two studies demonstrating a clinical benefit using radiation in combination with the anti-cytotoxic T-lymphocyte-associated protein 4 (CTLA-4) ICI antibody, Ipilimumab (Twyman-Saint Vistor, 2015).

Accordingly, an object of the presently disclosed invention is to provide therapies for the treatment of cancer using a HER3 targeting agent in combination with one or more immune checkpoint therapies, such as an ICI antibody.

Immune checkpoint therapies of the present invention include molecules that totally or partially reduce, inhibit, interfere with or modulate one or more checkpoint proteins. Checkpoint proteins regulate T-cell activation or function. Immune checkpoint therapies may unblock an existing immune response inhibition by binding to or otherwise disabling checkpoint inhibition. The immune checkpoint therapies may include monoclonal antibodies, humanized antibodies, fully human antibodies, antibody fragments, small molecule therapeutics, or any combination thereof.

Exemplary immune checkpoint therapies may specifically bind to and inhibit a checkpoint protein, such as the inhibitory receptors CTLA-4, PD-1, TIM-3, VISTA, BTLA, LAG-3 and TIGIT, and/or the activating receptors CD28, OX40, CD40, GITR, CD137, CD27, and HVEM. Additionally, the immune checkpoint therapy may bind to a ligand of any of the aforementioned checkpoint proteins, such as PD-L1, PD-L2, PD-L3, and PD-L4 (ligands for PD-1); CD80 and CD86 (ligands for CTLA-4); CD137-L (ligand of CD137); and GITR-L (ligand of GITR). Other exemplary immune checkpoint therapies may bind to checkpoint proteins such as CD226, B7-H3, B7-H4, BTLA, TIGIT, GALS, KIR, 2B4 (belongs to the CD2 family of molecules and is expressed on all NK, γδ, and memory CD8+ (αβ) T cells), CD160 (also referred to as BY55), and CGEN-15049.

Central to the immune checkpoint process are the CD137, CTLA-4 and PD-1 immune checkpoint pathways.

The CTLA-4 and PD-1 pathways are thought to operate at different stages of an immune response. CTLA-4 is considered the “leader” of the immune checkpoint inhibitors (ICI), as it stops potentially autoreactive T cells at the initial stage of naive T-cell activation, typically in lymph nodes. The PD-1 pathway regulates previously activated T cells at the later stages of an immune response, primarily in peripheral tissues. Moreover, progressing cancer patients have been shown to lack upregulation of PD-L1 by either tumor cells or tumor-infiltrating immune cells. Immune checkpoint therapies targeting the PD-1 pathway might thus be especially effective in tumors where this immune suppressive axis is operational and reversing the balance towards an immune protective environment would rekindle and strengthen a pre-existing anti-tumor immune response. PD-1 blockade can be accomplished by a variety of mechanisms including antibodies that bind PD-1 or its ligand, PD-L1.

According to certain aspects of the presently disclosed invention, the immune checkpoint therapy may include an inhibitor of the PD-1 checkpoint, which may decrease, block, inhibit, abrogate, or interfere with signal transduction resulting from the interaction of PD-1 with one or more of its binding partners, such as PD-L1 and PD-L2. The inhibitor of the PD-1 checkpoint may be an anti-PD-1 antibody, antigen binding fragment, fusion proteins, oligopeptides, and other molecules that decrease, block, inhibit, abrogate or interfere with signal transduction resulting from the interaction of PD-1 with PD-L1 and/or PD-L2. In some embodiments, a PD-1 checkpoint inhibitor reduces the negative co-stimulatory signal mediated by or through cell surface proteins expressed on T lymphocytes so as render a dysfunctional T-cell less dysfunctional (e.g., enhancing effector responses to antigen recognition). In some embodiments, the PD-1 checkpoint therapy is an anti-PD-1 antibody.

Thus, according to certain aspects of the present invention, the immune checkpoint therapy may include a monoclonal antibody against an immune checkpoint inhibitor (ICI) such as against CTLA-4, PD-1, or PD-L1.

According to certain aspects, the ICI antibody may be an antibody against PD-1. The ICI antibody may be an anti-PD-1 antibody, such as nivolumab. For example, the inhibitors of PD-1 biological activity (or its ligands) disclosed in U.S. Pat. No. 7,029,674. Exemplary antibodies against PD-1 include: Anti-mouse PD-1 antibody Clone J43 (Cat #BE0033-2) from BioXcell; Anti-mouse PD-1 antibody Clone RMP1-14 (Cat #BE0146) from BioXcell; mouse anti-PD-1 antibody Clone EH12; Merck's MK-3475 anti-mouse PD-1 antibody (Keytruda©, pembrolizumab, lambrolizumab); and AnaptysBio's anti-PD-1 antibody, known as ANB011; antibody MDX-1 106 (ONO-4538); Bristol-Myers Squibb's human IgG4 monoclonal antibody nivolumab (Opdivo®, BMS-936558, MDX1106); AstraZeneca's AMP-514, and AMP-224; and Pidilizumab (CT-011), CureTech Ltd.

According to certain aspects, the immune checkpoint therapy is an inhibitor of PD-L1. Exemplary inhibitors of PD-L1 include antibodies (e.g., an anti-PD-L1 antibody, i.e., ICI antibody), RNAi molecules (e.g., anti-PD-L1 RNAi), antisense molecules (e.g., an anti-PD-L1 antisense RNA), dominant negative proteins (e.g., a dominant negative PD-L1 protein), and small molecule inhibitors. An exemplary anti-PD-L1 antibody includes clone EH12. Exemplary antibodies against PD-L1 that may be used include: Genentech's MPDL3280A (RG7446); anti-mouse PD-L1 antibody Clone 10F.9G2 (Cat #BE0101) from BioXcell; anti-PD-L1 monoclonal antibody MDX-1105 (BMS-936559) and BMS-935559 from Bristol-Meyer's Squibb; MSB0010718C; mouse anti-PD-L1 Clone 29E.2A3; AstraZeneca's MEDI4736 (Durvalumab; Imfinzi®); and Atezolizumab (Tecentriq®).

According to certain aspects, the immune checkpoint therapy is an inhibitor of PD-L2 or may reduce the interaction between PD-1 and PD-L2. Exemplary inhibitors of PD-L2 include antibodies (e.g., an anti-PD-L2 antibody, i.e., ICI antibody), RNAi molecules (e.g., an anti-PD-L2 RNAi), antisense molecules (e.g., an anti-PD-L2 antisense RNA), dominant negative proteins (e.g., a dominant negative PD-L2 protein), and small molecule inhibitors. Antibodies include monoclonal antibodies, humanized antibodies, deimmunized antibodies, and Ig fusion proteins.

According to certain aspects, the immune checkpoint therapy may be an inhibitor of CTLA-4, such as an anti-CTLA-4 antibody, i.e., ICI antibody. According to one aspect, the ICI antibody may be ipilimumab. The anti-CTLA-4 antibody may block the binding of CTLA-4 to CD80 (B7-1) and/or CD86 (B7-2) expressed on antigen presenting cells. Exemplary antibodies against CTLA-4 include: Bristol Meyers Squibb's anti-CTLA-4 antibody ipilimumab (also known as Yervoy®, MDX-010, BMS-734016 and MDX-101); anti-CTLA4 Antibody, clone 9H10 from Millipore; Pfizer's tremelimumab (CP-675,206, ticilimumab); and anti-CTLA-4 antibody clone BNI3 from Abcam. According to certain aspects, the immune checkpoint inhibitor may be a nucleic acid inhibitor of CTLA-4 expression.

CD137 (also known “TNF receptor superfamily member 9”) is a costimulatory receptor member of the tumor necrosis factor receptor superfamily, mediating CD28-dependent and independent T-cell co-stimulation (Bartkowiak, 2015). CD137 is inducibly expressed by T cells, natural killer (NK) cells, dendritic cells (DC), B cells, and other cells of the immune system. The protein is composed of a 255-amino acid protein having a short N-terminal cytoplasmic portion, a transmembrane region, and an extracellular domain that possesses 3 cysteine-rich motifs. Ligation of CD137 by its ligand CD137L (4-1BBL; TNFSF9), which is mainly, though not exclusively, expressed on Antigen-Presenting Cells (APCs), evokes various T cell responses such as cell expansion, increased cytokine secretion and the prevention of activation-induced cell death. Thus, such ligation serves to activate the immune system. However, cis-interactions between CD137 and CD137L also potently downregulate the expression of CD137L (Kwon, 2015). The CD137 ligand thus functions to control the extent and kinetics of CD137-mediated immune system activation (Kwon, 2015). Significantly, CD137 expressed on human NK cells becomes upregulated upon binding to anti-tumor antibodies that have become bound to tumor cells (Wei, 2014).

Thus, according to certain aspects of the presently disclosed invention, the immune checkpoint therapy may include an antibody against CD137, which could be used to activate the immune system and thereby provide a therapy for cancer in combination with the presently disclosed HER3 targeting agents. Exemplary anti-CD137 antibodies that may be used are disclosed in U.S. Publication Nos. 20140274909; 20130280265; 20130273078; 20130071403; 20120058047; 20110104049; 20110097313; 20080166336; 20080019905; 20060188439; 20060182744; 20060121030; and 20030223989.

According to certain aspects of the present invention, the immune checkpoint therapy may include more than one modulator of an immune checkpoint protein. As such, the immune checkpoint therapy may include a first antibody or inhibitor against a first immune checkpoint protein and a second antibody or inhibitor against a second immune checkpoint protein.

D. DNA Damage Response Inhibitors

The additional agents administered in conjunction with the HER3 targeting agent may be one or more DNA damage response inhibitors (DDRi). DNA damage can be due to endogenous factors, such as spontaneous or enzymatic reactions, chemical reactions, or errors in replication, or may be due to exogenous factors, such as UV or ionizing radiation or genotoxic chemicals. The repair pathways that overcome this damage are collectively referred to as the DNA damage response or DDR. This signaling network acts to detect and orchestrate a cell's response to certain forms of DNA damage, most notably double strand breaks and replication stress. Following treatment with many types of DNA damaging drugs and ionizing radiation, cells are reliant on the DDR for survival. It has been shown that disruption of the DDR can increase cancer cell sensitivity to these DNA damaging agents and thus may improve patient responses to such therapies.

Within the DDR, there are several DNA repair mechanisms, including base excision repair, nucleotide excision repair, mismatch repair, homologous recombinant repair, and non-homologous end joining. Approximately 450 human DDR genes code for proteins with roles in physiological processes. Dysregulation of DDR leads to a variety of disorders, including genetic, neurodegenerative, immune, cardiovascular, and metabolic diseases or disorders and cancers. For example, the genes OGG1 and XRCC1 are part of the base excision repair mechanism of DDR, and mutations in these genes are found in renal, breast, and lung cancers, while the genes BRCA1 and BRCA2 are involved in homologous recombination repair mechanisms and mutations in these genes leads to an increased risk of breast, ovarian, prostate, pancreatic, as well as gastrointestinal and hematological cancers, and melanoma. Exemplary DDR genes are provided in Table 3.

An object of the presently disclosed invention is to administer radiolabeled HER3 targeting agents that deliver ionizing radiation in combination with a DDRi. Thus, according to certain aspects, the additional agent(s) administered with the HER3 targeting agent may target proteins in the DDR, i.e., DDR inhibitors or DDRi, thus maximizing DNA damage or inhibiting the repair if the damage, such as in G1 and S-phase and/or preventing repair in G2, ensuring the maximum amount of DNA damage is taken into mitosis, leading to cell death.

TABLE 3

| DNA repair | Gene | |
|---|---|---|
| mechanism | examples | Cancer |
| Base Excision | OGG1 | Renal, breast and lung cancer |
| Repair | XRCC1 | Non-small cell lung cancer |
| Nucleotide | ERCC1 | Lung and skin cancer, and glioma |
| Excision Repair | XP | Xeroderma pigmentosum predisposing |
| | | to skin cancer. Also increased risk of |
| | | bladder and lung cancer |
| Mismatch Repair | MSH2, | Lynch syndrome predisposing to |
| | MLH1 | colorectal cancer as well as endometrial, |
| | | ovarian, stomach, small intestine, |
| | | hepatobiliary tract, upper urinary tract, |
| | | brain and skin cancer |
| Homologous | BRCA1, | Increased risk of breast, ovarian, prostate, |
| Recombinant | BRCA2 | pancreatic, as well as gastrointestinal and |
| Repair | | hematological cancer, and melanoma |
| Non-homologous | KU70 | Breast, colorectal and lung cancer |
| End Joining | KU80 | Lung cancer |
| Cell cycle | ATM | Ataxia-telangiectasia predisposing to |
| checkpoints | | leukemia, breast and pancreatic cancer |
| | ATR | Leukemia, lymphoma, gastric and |
| | | endometrial cancer |

Moreover, one or more DDR pathways may be targeted to ensure cell death, i.e., lethality to the targeted cancer cells. For example, mutations in the BRCA1 and 2 genes alone may not be sufficient to ensure cell death, as other pathways, such as the PARP1 base excision pathway, may act to repair the DNA damage. Thus, combinations of multiple DDRi inhibitors or combining DDRi with antiangiogenic agents or immune checkpoint inhibitors, such as listed hereinabove, are possible and an object of the presently disclosed invention.

Exemplary DDRi—ATM and ATR Inhibitors

Ataxia telangiectasia mutated (ATM) and Ataxia talangiectasia mutated and Rad-3 related (ATR) are members of the phosphatidylinositol 3-kinase-related kinase (PIKK) family of serine/threonine protein kinases.

ATM is a serine/threonine protein kinase that is recruited and activated by DNA double-strand breaks. The ATM phosphorylates several key proteins that initiate activation of a DNA damage checkpoint, leading to cell cycle arrest, DNA repair, or cellular apoptosis. Several of these targets, including p53, CHK2, and H2AX, are tumor suppressors. The protein is named for the disorder ataxia telangiectasia caused by mutations of the ATM. The ATM belongs to the superfamily of phosphatidylinositol 3-kinase-related kinases (PIKKs), which includes six serine/threonine protein kinases that show a sequence similarity to a phosphatidylinositol 3-kinase (PI3K).

Like ATM, ATR is one of the central kinases involved in the DDR. ATR is activated by single stranded DNA structures, which may for example arise at resected DNA DSBs or stalled replication forks. When DNA polymerases stall during DNA replication, the replicative helicases continue to unwind the DNA ahead of the replication fork, leading to the generation of long stretches of single stranded DNA (ssDNA).

ATM has been found to assist cancer cells by providing resistance against chemotherapeutic agents and thus favors tumor growth and survival. Inhibition of ATM and/or ATR may markedly increase cancer cell sensitivity to DNA damaging agents, such as the ionizing radiation provided by the radiolabeled HER3 targeting agent. Accordingly, an object of the presently disclosed invention includes administration of an inhibitor of ATM (ATMi) and/or ATR (ATRi), in combination with the HER3 targeting agents, to inhibit or kill cancer cells, such as those expressing tor overexpressing HER3.

The inhibitor of ATM (ATMi) or ATR (ATRi) may be an antibody, peptide, or small molecule that targets ATM or ATR, respectively. Alternatively, an ATMi or ATRi may reduce or eliminate activation of ATM or ATR by one or more signaling molecules, proteins, or other compounds, or can result in the reduction or elimination of ATM or ATR activation by all signaling molecules, proteins, or other compounds. ATMi and/or ATRi also include compounds that inhibit their expression (e.g., compounds that inhibit ATM or ATR transcription or translation). An exemplary ATMi KU-55933 suppresses cell proliferation and induces apoptosis. Other exemplary ATMi include at least KU-59403, wortmannin, CP466722, and KU-60019. Exemplary ATRi include at least Schisandrin B, NU6027, NVP-BEA235, VE-821, VE-822, AZ20, and AZD6738.

Exemplary DDRi—Wee1 Inhibitors

The checkpoint kinase Wee1 catalyzes an inhibitory phosphorylation of both CDK1 (CDC2) and CDK2 on tyrosine 15, thus arresting the cell cycle in response to extrinsically induced DNA damage. Deregulated Wee1 expression or activity is believed to be a hallmark of pathology in several types of cancer. For example, Wee1 is often overexpressed in glioblastomas, malignant melanoma, hepatocellular carcinoma, breast cancer, colon carcinoma, lung carcinoma, and head and neck squamous cell carcinoma. Advanced tumors with an increased level of genomic instability may require functional checkpoints to allow for repair of such lethal DNA damage. As such, the present inventors believe that Wee1 represents an attractive target in advanced tumors where its inhibition is believed to result in irreparable DNA damage. Accordingly, an object of the presently disclosed invention includes administration of an inhibitor of Wee1, in combination with the HER3 targeting agents, to inhibit or kill cancer cells, such as those expressing tor overexpressing HER3.

A Wee1 inhibitor may be an antibody, peptide, or small molecule that targets Wee1. Alternatively, a Wee1 inhibitor may reduce or eliminate Wee1 activation by one or more signaling molecules, proteins, or other compounds, or can result in the reduction or elimination of Wee1 activation by all signaling molecules, proteins, or other compounds. The term also includes compounds that decrease or eliminate the activation or deactivation of one or more proteins or cell signaling components by Wee1 (e.g., a Wee1 inhibitor can decrease or eliminate Wee1-dependent inactivation of cyclin and Cdk activity). Wee1 inhibitors also include compounds that inhibit Wee1 expression (e.g., compounds that inhibit Wee1 transcription or translation).

Exemplary Wee1 inhibitors include AZD-1775 (i.e., adavosertib), and inhibitors such as those described in, e.g., U.S. Pat. Nos. 7,834,019; 7,935,708; 8,288,396; 8,436,004; 8,710,065; 8,716,297; 8,791,125; 8,796,289; 9,051,327; 9,181,239; 9,714,244; 9,718,821; and 9,850,247; U.S. Pub. Nos. US 20100113445 and 20160222459; and International Pub. Nos. WO2002090360, 2015019037, 2017013436, 2017216559, 2018011569, and 2018011570.

Further Wee1 inhibitors include a pyrazolopyrimidine derivative, a pyridopyrimidine, 4-(2-chlorophenyl)-9-hydroxypyrrolo[3,4-c]carbazole-1,3-(2H, 6H)-dione (CAS No. 622855-37-2), 6-butyl-4-(2-chlorophenyl)-9-hydroxypyrrolo[3,4-c]carbazole-1,3-(2H,6H)-dione (CAS No. 62285550-9), 4-(2-phenyl)-9-hydroxypyrrolo[3,4-c]carbazole-1,3-(2H,6H)-dione (CAS No. 1177150-89-8), and an anti-Wee1 small interfering RNA (siRNA) molecule.

Exemplary DDRi—PARP Inhibitors

Another exemplary type of DDRi that may be used are inhibitors of poly(ADP-ribose) polymerase (“PARP”). Inhibitors of the DNA repair protein PARP, referred to individually and collectively as “PARPi”, have been approved for use in a range of solid tumors, such as breast and ovarian cancer, particularly in patients having BRCA1/2 mutations. BRCA1 and 2 function in homologous recombination repair (HRR). When mutated, they induce genomic instability by shifting the DNA repair process from conservative and precise HRR to non-fidelitous methods such as DNA endjoining, which can produce mutations via deletions and insertions.

PARPi have been shown to exhibit synthetic lethality, as exhibited by potent single agent activity, in BRCA1/2 mutant cells. This essentially blocks repair of single-strand DNA breaks. Since HRR is not functional in these tumor cells, cell death results. Because most tumors do not carry BRCA1 or BRCA2 mutations, the potency of PARPi in such tumors is far less pronounced.

To date, the FDA has approved four PARPi drugs (olaparib, niraparib, rucaparib and talazoparib) as monotherapy agents, specifically in patients with germline and somatic mutations in the BRCA1 and BRCA2 genes. Along with veliparib, olaparib, niraparib and rucaparib were among the first generation of PARPi that entered clinical trials. Their IC50 values were found to be in the nanomolar range. In contrast, second generation PARPi like talazoparib have IC50 values in the picomolar range.

These PARPi all bind to the binding site of the cofactor, b nicotinamide adenine dinucleotide (b-NAD+), in the catalytic domain of PARP1 and PARP2. The PARP family of enzymes use NAD+ to covalently add Poly(ADP-ribose) (PAR) chains onto target proteins, a process termed “PARylation.” PARP1 (which is the best-studied member) and PARP2, are important components of the DNA damage response (DDR) pathway. PARP1 is involved in the repair of single-stranded DNA breaks, and possibly other DNA lesions (Woodhouse, et al.; Krishnakumar, et al.). Through its zinc finger domains, PARP1 binds to damaged DNA and then PARylates a series of DNA repair effector proteins, releasing nicotinamide as a by-product (Krishnakumar, et al.). Subsequently, PARP1 auto-PARylation leads to release of the protein from the DNA. The available PARPi, however, differ in their capability to trap PARP1 on DNA, which seems to correlate with cytotoxicity and drug efficacy. Specifically, drugs like talazoparib and olaparib are more effective in trapping PARP1 than are veliparib (Murai, et al., 2012; Murai, et al., 2014).

The efficacy of PARPi in ovarian cancer and breast cancer patients who have loss-of-function mutations in BRCA1 or BRCA2 genes is largely attributed to the genetic concept of synthetic lethality: that proteins of BRCA 1 and 2 normally maintain the integrity of the genome by mediating a DNA repair process, known as homologous recombination repair (HRR); and PARPi causes a persistent DNA lesion that, normally, would otherwise be repaired by HR. In the presence of PARPi, PARP1 is trapped on DNA which stalls progression of the replication fork. This stalling is cytotoxic unless timely repaired by the HR system. In cells lacking effective HR, they are unable to effectively repair these DNA lesions, and thus die.

Again, mutations in BRCA genes and others in the HRR system are not prevalent in many cancer types. So, to better harness the therapeutic benefits of PARPi in such cancers, one can induce “artificial” synthetic lethality by pairing a PARPi with either chemotherapy or radiation therapy. Preclinical studies have demonstrated that combining radiation therapy and PARPi can increase the sensitivity of BRCA1/2 mutant tumor cells to PARP inhibition and extend the sensitivity of non-mutant BRCA tumors to PARP inhibition. Additional studies have shown that ionizing radiation (TR) itself can mediate PARPi synthetic lethality in tumor cells.

Accordingly, an object of the presently disclosed invention is to administer radiolabeled HER3 targeting agents that deliver ionizing radiation in combination with a PARPi.

In the various embodiments of this invention, the PARPi may be any known agent performing that function, and preferably, one approved by the FDA. Preferably, the PARPi is olaparib (Lynparza®), niraparib (Zejula®), rucaparib (Rubraca®) or talazoparib (Talzenna®).

Clinically, therapy with PARPi has resulted in sustained anti-tumor responses in a range of cancers including ovarian, prostate, pancreatic, and triple-negative breast cancers (TNBC). In one clinical trial, TNBC patients with germline BRCA1/2 mutations were treated with the PARPi, olaparib. While this therapy demonstrated a higher disease stabilization rate in BRCA1/2-mutant compared to non-mutant patients, there were no sustained responses achieved in either cohort (Gelmon, 2011).

The present inventors realized that the effect of PARPi may be improved through increases in dsDNA breaks induced by ionizing radiation provided by a HER3 targeting agent while these repair pathways are being blocked by the PARPi. Exemplary PARPi include olaparib, niraparib, rucaparib and talazoparib.

E. CD47 Blockades

The additional agents administered with the HER3 targeting agent may be a CD47 blockade, such as any agent that interferes with, or reduces the activity and/or signaling between CD47 (e.g., on a target cell) and SIRPα (e.g., on a phagocytic cell) through interaction with either CD47 or SIRPα. Non-limiting examples of suitable CD47 blockades include CD47 and/or SIRPα reagents, including without limitation SIRPα polypeptides, anti-SIRPα antibodies, soluble CD47 polypeptides, and anti-CD47 antibodies or antibody fragments.

Additional examples of a CD47 blockade include agents that modulate the expression of CD47 and/or SIRPα. For example, such agents may include nucleic acid approaches such as phosphorodiamidate morpholino oligomers (PMO) that block translation of CD47 or antibodies specific for human CD47 that modulate, e.g., block, inhibit, reduce, antagonize, neutralize or otherwise interfere with CD47 expression. The CD47 antibodies or anti-sense approaches may inhibit CD47 expression (e.g., inhibiting cell surface expression of CD47), activity, and/or signaling, or may interfere with the interaction between CD47 and SIRPα. The agents provided herein completely or partially reduce or otherwise modulate CD47 expression or activity upon binding to, or otherwise interacting with, CD47, e.g., a human CD47. The reduction or modulation of a biological function of CD47 is complete, significant, or partial upon interaction between the antibodies and the human CD47 polypeptide and/or peptide. The agents are considered to inhibit CD47 expression or activity when the level of CD47 expression or activity in the presence of the antibody is decreased by at least 50%, e.g., by 60%, 70%, 80%, 90%, 95%, 96%, 98%, 99%, or 100% as compared to the level of CD47 expression or activity in the absence of interaction, e.g., binding, with the antibody described herein.

According to certain aspects, an anti-CD47 agent is an antibody that specifically binds CD47 (i.e., an anti-CD47 antibody) and reduces the interaction between CD47 on one cell (e.g., an infected cell) and SIRPα on another cell (e.g., a phagocytic cell). Non-limiting examples of suitable antibodies include clones B6H12, 5F9, 8B6, and C3 and any of those described in International Pub. No. WO2011/143624 and U.S. Pub. 20210246206. Suitable anti-CD47 antibodies include fully human, humanized or chimeric versions of such antibodies.

Exemplary human or humanized antibodies especially useful for in vivo applications in humans due to their low antigenicity include at least monoclonal antibodies against CD47, such as Hu5F9-G4, a humanized monoclonal antibody available from Gilead as Magrolimab (Sikic, et al. (2019) Journal of Clinical Oncology 37:946); Lemzoparlimab and TJC4 from I-Mab Biopharma; AO-176 from Arch Oncology, Inc; AK 117 from Akesobio Australia Pty; IMC-002 from Innovent Biologics; ZL-1201 from Zia Lab; SHR-1603 from Jiangsu HengRui Medincine Co.; and SRF231 from Surface Oncology. Bispecific monoclonal antibodies are also available, such as IBI-322, targeting both CD47 and PD-L1 from Innovent Biologics. Antibodies against SIRPα are also possible, such as ALX148 from Alx Oncology; BI 765063 (OSE-172) from OSE; as well as small molecule inhibitors, such as RRx-001 (1-bromoacetyl-3,3 dinitroazetidine) from EpicentRx and Azelnidipine (CAS number 123524-52-7) or pharmaceutically acceptable salts thereof. See also Table 4 for further description of exemplar agents.

TABLE 4

| Company | Approach | Agent/Program |
|---|---|---|
| Akesobio Australia | CD47 mAh | AK117 |
| Pty Ltd | | |
| Arch Oncology | CD47 mAh | AO-176 |
| (Tioma | | |
| Therapeutics) | | |
| Elpiscience | CD47 | ES004 |
| Biopharma Inc. | | |
| EpicentRx | Small molecule | RRx-001 |
| | inhibitor of | (1-bromoacetyl-3,3 |
| | dinitroazetidine | dinitroazetidine) |
| | hypoxia sensor | |
| | to downregulate | |
| | CD47/SIRPα | |
| ImmuneOncia | CD47 mAb human | IMC-002 |
| Therapeutics | | |
| Innovent | CD47 mAb | IBI-188 (CD47 mAb) |
| Biologies | CD47/PD-L1 | IBI-322 (Bispecific) |
| | bispecific mAb | |
| OSE | SIRPα mAb | BI 765063 (OSE-172) |
| Zai Lab | CD47 mAb | ZL-1201 |
| Alx Oncology | High-affinity | ALX148 |
| | SIRPα-Fc | |
| Gilead/Forty Seven | CD47 mAb | Magrolimab FSI-189 |
| | SIRPα mAb | |
| I-Mab Biopharma | CD47 mAb | TJC4 |
| Jiangsu HengRui | CD47 mAb | SHR-1603 |
| Medicine Co., Ltd. | | |
| Surface Oncology | CD47 mAb | SRF231 |
| | human | |
| Morphiex | CD47 targeting | MBT-001 |
| | phosphoro- | |
| | diamidate | |
| | morpholino | |
| | oligomers | |

AO-176, in addition to inducing tumor phagocytosis through blocking the CD47-SIRPα interaction, is reported to preferentially bind tumor cells versus normal cells (particularly RBCs where binding is negligible) and directly kills tumor versus normal cells.

According to certain aspects, a SIRPα reagent may include the portion of SIRPα that is sufficient to bind CD47 at a recognizable affinity, which normally lies between the signal sequence and the transmembrane domain, or a fragment thereof that retains the binding activity. A suitable SIRPα reagent reduces (e.g., blocks, prevents, etc.) the interaction between the native proteins SIRPα and CD47. For example, the CD47 blocking agent used in various aspects of the invention may be any of those disclosed in U.S. Pat. No. 9,969,789 including but not limited to the SIRPα-IgG Fc fusion proteins disclosed therein, such as TTI-621 and TTI-622, both of which preferentially bind CD47 on tumor cells while also engaging activating Fc receptors. A SIRPα-IgG Fc fusion protein including the amino acid sequence SEQ TD NO: 116, SEQ TD NO: 117, or SEQ TD NO: 118 may, for example, be used.

Therapeutically effective doses of an anti-CD47 antibody or other protein CD47 inhibitor may be a dose that leads to sustained serum levels of the protein of about 40 μg/ml or more (e.g., about 50 μg/ml or more, about 60 μg/ml or more, about 75 μg/ml or more, about 100 μg/ml or more, about 125 μg/ml or more, or about 150 μg/ml or more). Therapeutically effective doses or administration of a CD47 blockade, such as an anti-CD47 antibody or SIRPα fusion protein or small molecule, include, for example, amounts of 0.05-10 mg/kg (agent weight/subject weight), such as at least 0.1 mg/kg, 0.5 mg/kg, 1.0 mg/kg, 1.5 mg/kg, 2.0 mg/kg, 2.5 mg/kg, 3.0 mg/kg, 3.5 mg/kg, 4.0 mg/kg, 4.5 mg/kg, 5.0 mg/kg, 5.5 mg/kg, 6.0 mg/kg, 6.5 mg/kg, 7.0 mg/kg, 7.5 mg/kg, 8.0 mg/kg, 8.5 mg/kg, 9.0 mg/kg; or not more than 10 mg/kg, 9.5 mg/kg, 9.0 mg/kg, 8.5 mg/kg, 8.0 mg/kg, 7.5 mg/kg, 7.0 mg/kg, 6.5 mg/kg, 6.0 mg/kg, 5.5 mg/kg, 5.0 mg/kg, 4.5 mg/kg, 4.0 mg/kg, 3.5 mg/kg, 3.0 mg/kg, 2.5 mg/kg, 2.0 mg/kg, 1.5 mg/kg, 1.0 mg/kg, or any combination of these upper and lower limits. Therapeutically effective doses of a small molecule CD47 blockade such as those disclosed herein also, for example, include 0.01 mg/kg to 1,000 mg/kg and any subrange or value of mg/kg therein such as 0.01 mg/kg to 500 mg/kg or 0.05 mg/kg to 500 mg/kg, or 0.5 mg/kg to 200 mg/kg, or 0.5 mg/kg to 150 mg/kg, or 1.0 mg/kg to 100 mg/kg, or 10 mg/kg to 50 mg/kg.

According to certain aspects, the anti-CD47 agent is a soluble CD47 polypeptide that specifically binds SIRPα and reduces the interaction between CD47 on one cell (e.g., an infected cell) and SIRPα on another cell (e.g., a phagocytic cell). A suitable soluble CD47 polypeptide can bind SIRPα without activating or stimulating signaling through SIRPα because activation of SIRPα would inhibit phagocytosis. Instead, suitable soluble CD47 polypeptides facilitate the preferential phagocytosis of infected cells over non-infected cells. Those cells that express higher levels of CD47 (e.g., infected cells) relative to normal, non-target cells (normal cells) will be preferentially phagocytosed. Thus, a suitable soluble CD47 polypeptide specifically binds SIRPα without activating/stimulating enough of a signaling response to inhibit phagocytosis. In some cases, a suitable soluble CD47 polypeptide can be a fusion protein (for example, as described in U.S. Pub. No. 20100239579).

Advantageously, CD47 blockade can enhance the cytotoxic and prophagocytotic effect of a radiolabeled targeting agent, such as a radiolabeled HER3 and/or HER2 targeting agent, while reducing the dose-limiting radiotoxicity of the targeting agent, thereby improving tolerability and permitting higher radiation doses of the targeting agent to be used/tolerated in the treatment of a subject.

F. VEGF Inhibitors and VEFGR Inhibitors

The additional agents administered in combination or conjunction with the radiolabeled HER3 targeting agent, and optionally other agents and/or treatments as described herein, may, for example, include on or more VEGF inhibitors or VEGF-Receptor (VEGFR) inhibitors.

Such agents may, for example, include one or more of pazopanib (such as Votrient®); sunitinib (such as Sutent®); bevacizumab (such as Avastin®); sorafenib (such as Nexavar®); regorafenib (such as Stivarga®); cabozantinib (such as Cabometyx®); lenvatinib (such as Lenvima®); ponatinib (such as Iclusig®); cabozantinib (such as Cometriq®); ziv-aflibercept (such as Zaltrap®); axitinib (such as Inlyta®); 1-{2-chloro-4-[(6,7-dimethoxyquinolin-4-yl)oxy]phenyl}-3-(5-methylisoxazol-3-yl)urea (tivozanib) such as 1-{2-chloro-4-[(6,7-dimethoxyquinolin-4-yl)oxy]phenyl}-3-(5-methylisoxazol-3-yl)urea hydrochloride hydrate (tivozanib hydrochloride, such as Fotivda®); ramucirumab (such as Cyramza®); vandetanib (such as Caprelsa®); erlotinib (such as Tarceva®), dovitinib; vorolanib; ranibizumab; tarcocimab; emvododstat; muparfostat sodium; nintedanib (CBT-001; Cloudbreak Therapeutics LLC); Sevacizumab; Cediranib; famitinib L-malate; zanzalintinib; ibcasertib; lucitanib (e.g., as hydrochloride); sitravatinib; Ilorasertib; Tinengotinib; Tesevatinib; Olinvacimab; Acrizanib; brivanib alaninate; telatinib; and altiratinib, or any pharmaceutically acceptable salt of any of said agents such as but not limited to hydrochloride salts. For example, tivozanib inhibits phosphorylation of vascular endothelial growth factor receptor (VEGFR)-1, VEGFR-2 and VEGFR-3 and inhibits other kinases including c-kit and PDGFR β at clinically relevant concentrations

Cancers that may be treated by administration of a radiolabeled HER3 targeting agent in combination or conjunction with a VEGF and/or VEGFR inhibitor such as one or more of those disclosed herein, and optionally other agents or treatments as described herein, include, for example renal cell carcinoma (RCC), non-small cell lung cancer (NSCLC), hepatocellular carcinoma (HCC), gastrointestinal cancers such as colorectal carcinoma (CRC) and GIST cancers, breast cancer, tamoxifen-resistant breast cancer, metastatic breast cancer, triple-negative breast cancer, ovarian cancer, fallopian tube cancer, primary peritoneal cancer, bladder cancer, prostate cancer, advanced prostate cancer, metastatic prostate cancer, castration resistant prostate cancer, metastatic castration resistant prostate cancer, glioblastoma, gall bladder duct cancer, bile duct cancer, osteosarcoma, soft tissue sarcomas, or any of the cancers, such as any of the solid tumor cancers, disclosed herein. The cancers treated may, for example be non-metastatic, locally advanced, or metastatic. The cancer treated may, for example be relapsed and/or refractory (R/R). For example, the cancer treated may be relapsed or refractory RCC, such as metastatic or non-metastatic, in a human patient following one or more or two or more or three or more prior systemic therapies. In another embodiment, the patient treated using a radiolabeled HER3 targeting agent and a VEGF inhibitor and/or VEGFR inhibitor, such as tivozanib, has not been treated previously for the cancer with a radiolabeled HER3 targeting agent. In a related embodiment, the patient treated for the cancer using a radiolabeled HER3 targeting agent and a VEGF inhibitor and/or VEGFR inhibitor, such as tivozanib, has been previously treated with a VEGF inhibitor or VEGFR inhibitor, such as tivozanib, for the cancer and is relapsed and/or refractory with respect to said prior treatment but has not been previously treated with a radiolabeled HER3 targeting agent.

Dosages and administration schedules of the VEGF and/or VEGFR inhibitors that are used in combination or conjunction with a radiolabeled HER3 targeting agent in the treatment of a cancer such as any of those disclosed herein may, for example, include those prescribed or recommended for approved agents and/or those used in clinical trials of the agents. For example, for tivozanib (such as Fotivda®) 1.34 mg may be taken orally once daily for 21 days on treatment followed by 7 days off treatment for a 28-day cycle, in the treatment of a cancer such as renal cell carcinoma, such as R/R renal cell carcinoma. For patients with moderate hepatic impairment, the dose of tivozanib may, for example, be reduced to 0.89 mg for 21 days on treatment followed by 7 days off treatment (28-day cycle). Bevacizumab (such as Avastin®) may, for example, be dosed 5 mg/kg (patient weight) intravenously every 1, 2, 3, or 4 weeks, in the treatment of a cancer such as colorectal cancer, metastatic colorectal cancer (mCRC), RCC, metastatic RCC, NSCLC, ovarian cancer, cervical cancer, advanced cervical cancer, hepatocellular carcinoma, prostate cancer, castration-resistant prostate cancer (CRPC), locally advanced prostate cancer, metastatic prostate cancer, or metastatic CRPC (mCRPC).

One embodiment of the invention provides a method for treating a solid cancer in a mammalian subject, such as a human patient, the method including:

administering to the subject a therapeutically effective amount of a radionuclide labeled HER3 targeting agent; and

administering to the subject a therapeutically effective amount of at least one VEGF inhibitor or VEGFR inhibitor or pharmaceutically acceptable salt thereof,

wherein the radionuclide labeled HER3 targeting agent includes a radionuclide labeled monoclonal antibody, for example, any of those disclosed herein, such as a radionuclide labeled antibody including:
(i) one or both of
   (a) an immunoglobulin heavy chain variable region including a CDR-H1 including SEQ ID NO:15, a CDR-H2 including SEQ ID NO:16, and/or a CDR-H3 including SEQ ID NO:17, and
   (b) an immunoglobulin light chain variable region including a CDR-L1 including SEQ ID NO:18, a CDR-L2 including SEQ ID NO:19, and/or a CDR-L3 including SEQ ID NO:20;
(ii) one or both of an immunoglobulin heavy chain variable region including SEQ ID NO:21 and an immunoglobulin light chain variable region including SEQ ID NO:22;
(iii) one or both of an immunoglobulin heavy chain amino acid sequence of SEQ ID NO:23 and an immunoglobulin light chain amino acid sequence of SEQ ID NO:24;
(iv) one or both of an immunoglobulin heavy chain sequence including SEQ ID NO:77 and an immunoglobulin light chain sequence including SEQ ID NO:78; or
(v) AV-203, and

wherein the at least one VEGF inhibitor or VEGFR inhibitor includes one or more of pazopanib; sunitinib; bevacizumab; sorafenib; regorafenib; cabozantinib; lenvatinib; ponatinib; cabozantinib; ziv-aflibercept; axitinib; tivozanib (such as tivozanib hydrochloride), ramucirumab; vandetanib; erlotinib, dovitinib; vorolanib; ranibizumab; tarcocimab; emvododstat; muparfostat (such as muparfostat sodium); nintedanib; Sevacizumab; Cediranib; famitinib (such as famitinib L-malate); zanzalintinib; ibcasertib; lucitanib (such as lucitanib hydrochloride); sitravatinib; Ilorasertib; Tinengotinib; Tesevatinib; Olinvacimab; Acrizanib; brivanib (such as brivanib alaninate); telatinib; and altiratinib.

The radionuclide labeled HER3 targeting agent in this embodiment may, for example, include a radionuclide selected from ⁶⁴Cu, ⁶⁷Cu, ²⁰¹T1, ⁴⁷Sc, ⁹⁰Y, ¹⁷⁷Lu, ¹⁸⁶Re, ¹⁸⁸Re, ¹⁵³Sm, ³²p ²²⁵Ac, ²¹³Bi, ²¹³Po, ²¹¹At, ²¹²Bi, ²¹³Bi, ²²³Ra, ²²⁷Th, ¹⁴⁹Tb, ¹³⁷Cs, ²¹²Pb or ¹⁰³Pd, or any of those disclosed herein, or any combination thereof.

The solid cancer in this embodiment may, for example, be renal cell carcinoma (RCC), metastatic RCC, non-small cell lung cancer (NSCLC), hepatocellular carcinoma (HCC), colorectal carcinoma (CRC), gastric cancer GIST cancer, breast cancer, tamoxifen-resistant breast cancer, metastatic breast cancer, triple-negative breast cancer, ovarian cancer, fallopian tube cancer, primary peritoneal cancer, bladder cancer, prostate cancer, advanced prostate cancer, metastatic prostate cancer, castration resistant prostate cancer, metastatic castration resistant prostate cancer, gall bladder duct cancer, bile duct cancer, osteosarcoma, soft tissue sarcoma, or pancreatic cancer, a non-metastatic form or metastatic form of any of the aforementioned cancers, or a relapsed and/or refractory form of any of the preceding.

### EXAMPLES

### Example 1: Production of Radiolabeled HER3 Targeting Agent

The HER3 targeting agent, such as a monoclonal antibody against HER3, may be labeled with Indium-111 (¹¹¹In) or Actinium-225 (²²⁵Ac) according to procedures detailed in International Publication No. WO 2017/155937 and U.S. Provisional Patent Application No. 63/042,651 filed Dec. 9, 2019 titled “Compositions and methods for preparation of site-specific radioconjugates.”

Radiolabeling: As example, the antibody may be conjugated to a chelator-bearing linker, for example, as described herein or in the preceding patent applications. An exemplary linker includes at least dodecane tetraacetic acid (DOTA), wherein a goal of the conjugation reaction is to achieve a DOTA-antibody ratio of 3:1 to 5:1. Chelation with the radionuclide ¹¹¹In or ²²⁵Ac may then be performed and efficiency and purity of the resulting ¹¹¹In- or ²²⁵Ac-labeled anti-HER3 antibody may be determined by HPLC and iTLC.

An exemplary labeling reaction for ²²⁵Ac is as follows: A reaction including 15 μl 0.15 M NH₄OAc buffer, pH=6.5 and 2 μL (10 μg) DOTA-anti-HER3 (5 mg/ml) may be mixed in an Eppendorf reaction tube, and 4 μL ²²⁵Ac (10 μCi) in 0.05 M HCl subsequently added. The contents of the tube may be mixed with a pipette tip and the reaction mixture incubated at 37° C. for 90 min with shaking at 100 rpm. At the end of the incubation period, 3 μL of a 1 mM DTPA solution may be added to the reaction mixture and incubated at room temperature for 20 min to bind the unreacted ²²⁵Ac into the ²²⁵Ac-DTPA complex. Instant thin layer chromatography with 10 cm silica gel strip and 10 mM EDTA/normal saline mobile phase may be used to determine the radiochemical purity of ²²⁵Ac-DOTA-anti-HER3 through separating ²²⁵Ac-labeled anti-HER3 (²²⁵Ac-DOTA-anti-HER3) from free ²²⁵Ac (²²⁵Ac-DTPA). In this system, the radiolabeled antibody stays at the point of application and ²²⁵Ac-DTPA moves with the solvent front. The strips may be cut in halves and counted in the gamma counter equipped with the multichannel analyzer using channels 72-110 for ²²⁵Ac to exclude its daughters.

Purification: An exemplary radiolabeled HER3 targeting agent, such as ²²⁵Ac-DOTA-anti-HER3, may be purified either on PD10 columns pre-blocked with 1% HSA or on Vivaspin centrifugal concentrators with a 50 kDa MW cut-off with 2×1.5 mL washes, 3 min per spin. HPLC analyses of the ²²⁵Ac-DOTA-anti-HER3 after purification may be conducted using a Waters HPLC system equipped with flow-through Waters UV and Bioscan Radiation detectors, using a TSK3000SW XL column eluted with PBS at pH=7.4 and a flow rate of 1 ml/min.

Stability determination: An exemplary radiolabeled HER3 targeting agent, such as 225Ac-DOTA-anti-HER3, may be used for stability determination, wherein the ²²⁵Ac-DOTA-anti-HER3 may be tested either in the original volume or diluted (2-10 fold) with the working buffer (0.15 M NH₄OAc) and incubated at room temperature (rt) for 48 hours or at 4° C. for 96 hours and tested by ITLC. Stability is determined by comparison of the intact radiolabeled anti-HER3 before and after incubation. Other antibodies labeled with ²²⁵Ac have been found to be stable at 4° C. for up to 96 hrs.

Immunoreactivity (IR) determination: An exemplary radiolabeled HER3 targeting agent, such as ²²⁵Ac-DOTA-anti-HER3, may be used in immunoreactivity experiments. HER3 positive cells and control HER3 negative cells may be used in the amounts of 1.0-7.5 million cells per sample to investigate the amount of binding (percent radioactivity binding to cells after several washes; or using an immunoreactive fraction (IRF) bead assay may be performed according to methods disclosed in as described by Sharma, 2019). Prior assays for other antibodies radiolabeled with ¹¹¹In or ²²⁵Ac demonstrated about 50-60% immunoreactivity.

### Example 2—Exemplary PARPi Administration and Dosing Regimes

(A) Olaparib (Lynparza®)—Normal and Reduced Dosing Regimens

Olaparib is sold by AstraZeneca under the brand name Lynparza®. Lynparza® is sold in tablet form at 100 mg and 150 mg. The dosage is 300 mg taken orally twice daily for a daily total of 600 mg. Dosing continues until disease progression or unacceptable toxicity. This dosing regimen is referred to herein as the “normal” human dosing regimen for Lynparza®, regardless of the disorder treated. Any dosing regimen having a shorter duration (e.g., 21 days) or involving the administration of less Lynparza® (e.g., 300 mg/day) is referred to herein as a “reduced” human dosing regimen. Examples of reduced human dosing regimens include the following: (i) 550 mg/day; (ii) 500 mg/day; (iii) 450 mg/day; (iv) 400 mg/day; (v) 350 mg/day; (vi) 300 mg/day; (vii) 250 mg/day; (viii) 200 mg/day; (ix) 150 mg/day; (x) 100 mg/day; or (xi) 50 mg/day.

(B) Niraparib (Zejula®)—Normal and Reduced Dosing Regimens

Niraparib is sold by Tesaro under the brand name Zejula®. Zejula® is sold in capsule form at 100 mg. The dosage is 300 mg taken orally once daily. Dosing continues until disease progression or unacceptable adverse reaction. This dosing regimen is referred to herein as the “normal” human dosing regimen for Zejula®, regardless of the disorder treated. Any dosing regimen having a shorter duration (e.g., 21 days) or involving the administration of less Zejula® (e.g., 150 mg/day) is referred to herein as a “reduced” human dosing regimen. Examples of reduced human dosing regimens include the following: (i) 250 mg/day; (ii) 200 mg/day; (iii) 150 mg/day; (iv) 100 mg/day; or (v) 50 mg/day.

(C) Rucaparib (Rubraca®)—Normal and Reduced Dosing Regimens

Rucaparib is sold by Clovis Oncology, Inc. under the brand name Rubraca™. Rubraca™ is sold in tablet form at 200 mg and 300 mg. The dosage is 600 mg taken orally twice daily for a daily total of 1,200 mg. Dosing continues until disease progression or unacceptable toxicity. This dosing regimen is referred to herein as the “normal” human dosing regimen for Rubraca™, regardless of the disorder treated. Any dosing regimen having a shorter duration (e.g., 21 days) or involving the administration of less Rubraca™ (e.g., 600 mg/day) is referred to herein as a “reduced” human dosing regimen. Examples of reduced human dosing regimens include the following: (i) 1,150 mg/day; (ii) 1,100 mg/day; (iii) 1,050 mg/day; (iv) 1,000 mg/day; (v) 950 mg/day; (vi) 900 mg/day; (vii) 850 mg/day; (viii) 800 mg/day; (ix) 750 mg/day; (x) 700 mg/day; (xi) 650 mg/day; (xii) 600 mg/day; (xiii) 550 mg/day; (xiv) 500 mg/day; (xv) 450 mg/day; (xvi) 400 mg/day; (xvii) 350 mg/day; (xviii) 300 mg/day; (xix) 250 mg/day; (xx) 200 mg/day; (xxi) 150 mg/day; or (xxii) 100 mg/day.

(D) Talazoparib (Talzenna™)—Normal and Reduced Dosing Regimens

Talazoparib is sold by Pfizer Labs under the brand name Talzenna™. Talzenna™ is sold in capsule form at 1 mg. The dosage is 1 mg taken orally. Dosing continues until disease progression or unacceptable toxicity. This dosing regimen is referred to herein as the “normal” human dosing regimen for Talzenna™, regardless of the disorder treated. Any dosing regimen having a shorter duration (e.g., 21 days) or involving the administration of less Talzenna™ (e.g., 0.5 mg/day) is referred to herein as a “reduced” human dosing regimen. Examples of reduced human dosing regimens include the following: (i) 0.9 mg/day; (ii) 0.8 mg/day; (iii) 0.7 mg/day; (iv) 0.6 mg/day; (v) 0.5 mg/day; (vi) 0.4 mg/day; (vii) 0.3 mg/day; (viii) 0.2 mg/day; or (ix) 0.1 mg/day.

### Example 3: Dosing Regimens for HER3 Targeting Agent and PARPi

A human patient may be treated according to the following regimen. One of olaparib, niraparib, rucaparib or talazoparib (PARPi) is orally administered according to one of the dosing regimens listed in Example 2, accompanied by intravenous administration of a radiolabeled HER3 targeting agent as detailed herein in either single or fractional administration. For example, the dosing regimens include, by way of example: (a) the PARPi and the HER3 targeting agent administered concurrently, wherein (i) each is administered beginning on the same day, (ii) the HER3 targeting agent is administered in a single dose or fractionated doses not less than one week apart, and (iii) the PARPi is administered daily or twice daily (as appropriate), and for a duration equal to or exceeding that of the HER3 targeting agent administration; or (b) the PARPi and HER3 targeting agent are administered concurrently, wherein (i) the PARPi administration precedes HER3 targeting agent administration by at least one week, (ii) the HER3 targeting agent is administered in a single dose or fractionated doses not less than one week apart, and (iii) the PARPi is administered daily or twice daily (as appropriate), and for a duration equal to or exceeding that of the HER3 targeting agent administration.

### Example 4: Dosing Regimens for HER3 Targeting Agent and a CD47 Blockade

According to certain aspects of the present invention, the CD47 blocking agent may, for example, be a monoclonal antibody that prevents CD47 binding to SIRPα. Exemplary protein CD47 blockades include magrolimab, lemzoparlimab, AO-176, TTI-621, TTI-622, or any combination thereof. The CD47 blockade may alternatively, or additionally, include agents that modulate the expression of CD47 and/or SIRPα, such as phosphorodiamidate morpholino oligomers (PMO) that block translation of CD47 such as MBT-001 (PMO, morpholino, Sequence: 5′-CGTCACAGGCAGGACCCACTGCCCA-3′) [SEQ ID NO:114]) or any of the PMO oligomer CD47 inhibitors disclosed in any of U.S. Pat. Nos. 8,557,788, 8,236,313, 10,370,439 and Int'l Pub. No. WO2008060785. Therapeutically effective doses of anti-CD47 antibodies include at least 0.05-10 mg/kg. Thus, methods of the present invention may include administering one or more of the anti-CD47 antibodies or other agents, accompanied by intravenous administration of a radiolabeled HER3 targeting agent as detailed herein in either single or fractional administration. For example, the dosing regimens include, by way of example: (a) the anti-CD47 antibody or agent and the HER3 targeting agent administered concurrently, wherein (i) each is administered beginning on the same day, (ii) the HER3 targeting agent is administered in a single dose or fractionated doses not less than one week apart, and (iii) the anti-CD47 antibody or agent is administered daily or twice daily (as appropriate), and for a duration equal to or exceeding that of the HER3 targeting agent administration; or (b) the anti-CD47 antibody or agent and HER3 targeting agent are administered concurrently, wherein (i) the anti-CD47 antibody or agent administration precedes HER3 targeting agent administration by at least one week, (ii) the HER3 targeting agent is administered in a single dose or fractionated doses not less than one week apart, and (iii) the anti-CD47 antibody or agent is administered daily or twice daily (as appropriate), and for a duration equal to or exceeding that of the HER3 targeting agent administration.

### Example 5: Dosing Regimens for HER3 Targeting Agent and an ICI

According to certain aspects of the present invention, the immune checkpoint inhibitor (ICI) may be a monoclonal antibody against any of PD-1, PD-L1, PD-L2, CTLA-4, CD137. Therapeutically effective doses of these antibodies include at least 0.05-10 mg/kg. Thus, method of the present invention include administering one or more ICI, accompanied by intravenous administration of a radiolabeled HER3 targeting agent as detailed herein in either single or fractional administration. For example, the dosing regimens include, by way of example: (a) the ICI and the HER3 targeting agent administered concurrently, wherein (i) each is administered beginning on the same day, (ii) the HER3 targeting agent is administered in a single dose or fractionated doses not less than one week apart, and (iii) the ICI is administered daily or twice daily (as appropriate), and for a duration equal to or exceeding that of the HER3 targeting agent administration; or (b) the ICI and HER3 targeting agent are administered concurrently, wherein (i) the anti-CD47 antibody administration precedes HER3 targeting agent administration by at least one week, (ii) the HER3 targeting agent is administered in a single dose or fractionated doses not less than one week apart, and (iii) the ICI is administered daily or twice daily (as appropriate), and for a duration equal to or exceeding that of the HER3 targeting agent administration.

Without limitation, the following aspects are also provided by this disclosure:

Aspect 1. A method for treating a solid cancer in a mammalian subject such as a human patient, the method including: administering to the subject a therapeutically effective amount of a radiolabeled HER3 targeting agent.

Aspect 2. The method according to any preceding aspect, wherein the solid cancer is a breast cancer, gastric cancer, bladder cancer, cervical cancer, endometrial cancer, skin cancer, stomach cancer, testicular cancer, esophageal cancer, bronchioloalveolar cancer, prostate cancer, colorectal cancer, ovarian cancer, cervical epidermoid cancer, pancreatic cancer, lung cancer, renal cancer, head and neck cancer, or any combination thereof.

Aspect 3. The method according to any preceding aspect, wherein the solid cancer is colorectal cancer, gastric cancer, ovarian cancer, non-small cell lung carcinoma, head and neck squamous cell cancer, pancreatic cancer, renal cancer, or any combination thereof.

Aspect 4. The method according to any preceding aspect, wherein the solid cancer is a HER3-positive cancer such as a HER3-positive solid tumor.

Aspect 5. The method according to any preceding aspect, wherein the radiolabeled HER3 targeting agent includes a radiolabel selected from ¹³¹I, ¹²⁵I, ¹²³I, ⁹⁰Y ¹⁷⁷Lu, ¹⁸⁶Re, ¹⁸⁸Re, ⁸⁹Sr, ¹⁵³Sm, ³²P, ²²⁵Ac, ²¹³Bi, ²¹³Po, ²¹¹At, ²¹²Bi, ²¹³Bi, ²²³Ra, ²²⁷Th, ¹⁴⁹Tb, ¹³⁷Cs, ²¹²Pb, ¹⁰³Pd, or any of those disclosed herein, or any combination thereof.

Aspect 6. The method according to any preceding aspect, wherein the radiolabeled HER3 targeting agent includes a radiolabel selected from ¹³¹I, ⁹⁰Y, ¹⁷⁷Lu, ²²⁵Ac, ²¹³Bi, ²¹¹At, ²¹³Bi, ²²⁷Th, ²¹²Pb, or any combination thereof.

Aspect 7. The method according to any preceding aspect, wherein the radiolabeled HER3 targeting agent includes an antibody against HER3.

Aspect 8. The method according to any preceding aspect, wherein the HER3 targeting agent includes an anti-HER3 monoclonal antibody such as any of those disclosed herein, such as a HER3 antibody selected from Patritumab, Seribantumab (MM-121), Lumretuzumab, Elgemtumab, GSK2849330, and AV-203 and any combination thereof.

Aspect 9. The method according to any preceding aspect, wherein the HER3 targeting agent includes a monoclonal antibody: (i) having a heavy chain sequence including SEQ ID NO:77 and/or a light chain sequence including SEQ ID NO:78; (ii) having an immunoglobulin heavy chain variable region including a CDR-H1 including SEQ ID NO:15, a CDR-H2 including SEQ ID NO:16, and/or a CDR-H3 including SEQ ID NO:17, and/or an immunoglobulin light chain variable region including a CDR-L1 including SEQ ID NO:18, a CDR-L2 including SEQ ID NO:19, and/or a CDR-L3 including SEQ ID NO:20; (iii) having an immunoglobulin heavy chain variable region including SEQ ID NO:21 and/or an immunoglobulin light chain variable region including SEQ ID NO:22; or (iv) having an immunoglobulin heavy chain amino acid sequence of SEQ ID NO:23 and/or an immunoglobulin light chain amino acid sequence of SEQ ID NO:24.

Aspect 10. The method according to any preceding aspect, wherein the HER3 targeting agent includes a monoclonal antibody including a heavy chain variable region having an amino acid sequence as set forth in SEQ. ID NO:7 and/or a light chain variable region having an amino acid sequence as set forth in SEQ. ID NO:8.

Aspect 11. The method according to any preceding aspect, wherein the HER3 targeting agent includes a monoclonal antibody including one or more of the heavy chain N-terminal region and complementarity determining regions (CDRs) having amino acid sequences as set forth in SEQ. ID NO:13 and/or 1-3, respectively; and/or including one or more of the light chain N-terminal region and CDRs having amino acid sequences as set forth in SEQ. ID NO:14 and/or 4-6, respectively.

Aspect 12. The method according to any preceding aspect, wherein the effective amount of the radiolabeled HER3 targeting agent is a maximum tolerated dose.

Aspect 13. The method according to any preceding aspect, wherein the radiolabeled HER3 targeting agent is ²²⁵Ac-, ¹⁷⁷Lu-, or ¹³¹I-labeled.

Aspect 14. The method according to any preceding aspect, wherein the therapeutically effective amount of the radiolabeled HER3 targeting agent includes a single dose that delivers less than 2Gy, or less than 8 Gy, such as doses of 2 Gy to 8 Gy, to the subject.

Aspect 15. The method according to any preceding aspect, wherein the radiolabeled HER3 targeting agent is ²²⁵Ac-labeled, and the effective amount of the ²²⁵Ac-labeled HER3 targeting agent includes a dose of 0.1 to 50 uCi/kg body weight of the subject, or 0.2 to 20 uCi/kg body weight of the subject, or 0.5 to 10 uCi/kg subject body weight.

Aspect 16. The method according to any preceding aspect, wherein the radiolabeled HER3 targeting agent is a full-length antibody against HER3 that is ²²⁵Ac-labeled, and the effective of the ²²⁵Ac-labeled HER3 targeting agent includes less than 5 uCi/kg body weight of the subject, such as 0.1 to 5 uCi/kg body weight of the subject.

Aspect 17. The method according to any one of aspects 1 to 6, wherein the radiolabeled HER3 targeting agent is an antibody fragment, such as a minibody or nanobody against HER3 that is ²²⁵Ac-labeled, and the effective of the ²²⁵Ac-labeled HER3 targeting agent includes greater than 5 uCi/kg body weight of the subject, such as 5 to 20 uCi/kg body weight of the subject.

Aspect 18. The method according to any one of aspects 1 to 14, wherein the radiolabeled HER3 targeting agent is ²²⁵Ac-labeled, and the effective amount of the ²²⁵Ac-labeled HER3 targeting agent includes 2 μCi to 2 mCi, or 2 μCi to 250 μCi, or 75 μCi to 400 μCi.

Aspect 19. The method according to any one of aspects 1 to 14, wherein the radioisotope labeled HER3 targeting agent is ¹⁷⁷Lu-labeled and the effective amount of the HER3 targeting agent includes a dose of less than 1000 uCi/kg body weight of the subject, such as a dose of 1 to 900 uCi/kg body weight of the subject, or 5 to 250 uCi/kg body weight of the subject or 50 to 450 uCi/kg body weight.

Aspect 20. The method according to any one of aspects 1 to 14, wherein the radioisotope labeled HER3 targeting agent is ¹⁷⁷Lu-labeled, and the effective amount of the ¹⁷⁷Lu-labeled HER3 targeting agent includes a dose of 10 mCi to at or below 30 mCi, or from at least 100 Ci to at or below 3 mCi, or from 3 mCi to at or below 30 mCi.

Aspect 21. The method according to any one of aspects 1 to 14, wherein the radiolabeled HER3 targeting agent is ¹³¹I-labeled, and the effective amount of the ¹³¹I-labeled HER3 targeting agent includes a dose of less than or at 1200 mCi, such as a dose of 25 to 1200 mCi, or 100 to 400 mCi, or 300 to 600 mCi, or 500 to 1000 mCi.

Aspect 22. The method according to any one of aspects 1 to 14, wherein the radiolabeled HER3 targeting agent is ¹³¹I-labeled, and the effective amount of the ¹³¹I-labeled HER3 targeting agent includes a dose of less than or at 200 mCi, such as a dose of 1 to 200 mCi, or 25 to 175 mCi, or 50 to 150 mCi.

Aspect 23. The method according to any preceding aspect, wherein the effective amount of the HER3 targeting agent includes a protein dose of less than 3 mg/kg body weight of the subject, such as from 0.001 mg/kg patient weight to 3.0 mg/kg patient weight, or from 0.005 mg/kg patient weight to 2.0 mg/kg patient weight, or from 0.01 mg/kg patient weight to 1 mg/kg patient weight, or from 0.1 mg/kg patient weight to 0.6 mg/kg patient weight, or 0.3 mg/kg patient weight, or 0.4 mg/kg patient weight, or 0.5 mg/kg patient weight, or 0.6 mg/kg patient weight.

Aspect 24. The method according to any preceding aspect, wherein the HER3 targeting agent is administered according to a dosing schedule selected from the group consisting of once every 7, 10, 12, 14, 20, 24, 28, 36, and 42 days throughout a treatment period, wherein the treatment period includes at least two doses.

Aspect 25. The method according to any one of aspects 1 to 6, wherein the HER3 targeting agent is a peptide or small molecule.

Aspect 26. The method according to any preceding aspect, further including administering to the subject a therapeutically effective amount of an immune checkpoint therapy, a chemotherapeutic agent, a DNA damage response inhibitor (DDRi), a CD47 blockade, a VEGF inhibitor, a VEGFR inhibitor, or any combination thereof.

Aspect 27. The method according to aspect 26, wherein the immune checkpoint therapy includes an antibody or other blocking agent against CTLA-4, PD-1, TIM-3, VISTA, BTLA, LAG-3, TIGIT, CD28, OX40, GITR, CD137, CD40, CD40L, CD27, HVEM, PD-L1, PD-L2, PD-L3, PD-L4, CD80, CD86, CD137-L, GITR-L, CD226, B7-H₃, B7-H4, BTLA, TIGIT, GALS, KIR, 2B4, CD160, or CGEN-15049, or any combination of such antibodies and blocking agents.

Aspect 28. The method according to aspect 27, wherein the immune checkpoint therapy includes one or more antibodies against PD-1, PD-L1, PD-L2, CTLA-4, CD137, or any combination thereof.

Aspect 29. The method according to aspect 26, wherein the DDRi includes a poly(ADP-ribose) polymerase inhibitor (PARPi), an ataxia telangiectasia mutated inhibitor (ATMi), an ataxia talangiectasia mutated and Rad-3 related inhibitor (ATRi), or a Wee1 inhibitor.

Aspect 30. The method according to aspect 29, wherein the PARPi includes one or more of olaparib, niraparib, rucaparib and talazoparib.

Aspect 31. The method according to aspect 29, wherein the ATMi includes one or more of KU-55933, KU-59403, wortmannin, CP466722, or KU-60019.

Aspect 32. The method according to aspect 29, wherein the ATRi includes one or more of Schisandrin B, NU6027, NVP-BEA235, VE-821, VE-822, AZ20, or AZD6738.

Aspect 33. The method according to aspect 29, wherein the Wee1 inhibitor includes AZD-1775 (i.e., adavosertib).

Aspect 34. The method according to aspect 26, wherein the CD47 blockade includes an agent, such as a monoclonal antibody that prevents CD47 binding to SIRPα and/or an agent that modulates CD47 expression.

Aspect 35: The method according to aspect 34, wherein the CD47 blockade includes one or more of magrolimab, lemzoparlimab, AO-176, TTI-621, TTI-622, or any combination thereof, and/or wherein the agent that modulates CD47 expression includes phosphorodiamidate morpholino oligomers (PMO) that reduce expression of CD47 (e.g., MBT-001).

Aspect 36: The method according to aspect 34, wherein the therapeutically effective amount of the CD47 blockade includes 0.05 to 5 mg/Kg patient weight.

Aspect 37. The method according to aspect 26, wherein the HER3 targeting agent is administered at least one week before the immune checkpoint therapy and/or the DDRi and/or the CD47 blockade; or wherein the immune checkpoint therapy and/or the DDRi and/or CD47 blockade is administered at least one week before the HER3 targeting agent.

Aspect 38. The method according to aspect 26, wherein the HER3 targeting agent is administered with one of the immune checkpoint therapy or the DDRi or the CD47 blockade, and the other of the immune checkpoint therapy or the DDRi or the CD47 blockade is administered either before or after the HER3 targeting agent.

Aspect 39. The method according to aspect 26, wherein the HER3 targeting agent is administered simultaneously with the immune checkpoint therapy and/or the DDRi and/or the CD47 blockade.

Aspect 40. The method according to any preceding aspect, wherein the HER3 targeting agent is a multi-specific antibody, wherein the multi-specific antibody includes: a first target recognition component which specifically binds to an epitope of HER3, and a second target recognition component which specifically binds to a different epitope of HER3 than the first target recognition component, or an epitope of a different antigen.

Aspect 41. The method according to aspect 40, wherein the HER3 targeting agent includes a bispecific antibody against HER3/HER2 such as MM-111 or MCLA0-128, or against IGF-1R/IER3 such as MM-141 (i.e., Istiratumab), and/or against HER1/HER3 such as MEHD7945A (i.e., Duligotumab).

Aspect 42. A method for treating a proliferative disease or disorder, the method including: diagnosing the subject with HER3-positive cells; and if the subject has HER3-positive cells, administering to the subject a therapeutically effective amount of an HER3 targeting agent according to any of the methods of aspects 1 to 41.

Aspect 43. The method according to aspect 42, wherein the diagnosing includes obtaining a sample of blood or tissue from the subject; mounting the sample on a substrate; and detecting the presence or absence of HER3 antigen using a diagnostic antibody, wherein the diagnostic antibody includes an antibody against HER3 labeled with a radiolabel such as ³H, ¹⁴C, ³²p ³⁵S and ¹²⁵⁷I; fluorescent or chemiluminescent compounds, such as fluorescein isothiocyanate, rhodamine, or luciferin; or an enzyme, such as alkaline phosphatase, β-galactosidase, or horseradish peroxidase.

Aspect 44. The method according to aspect 42, wherein the diagnosing includes administering a HER3 targeting agent to the subject, wherein the HER3 targeting agent includes a radiolabel selected from the group including ¹⁸F, ¹¹C, ⁶⁸Ga, ⁶⁴Cu, ⁸⁹Zr, ¹²⁴, ^{99m}Tc, or ¹¹¹In; waiting a time sufficient to allow the HER3 targeting agent to accumulate at a tissue site; and imaging the tissues with a non-invasive imaging technique to detect presence or absence of HER3-positive cells.

Aspect 45. The method according to aspect 44, wherein the non-invasive imaging technique includes positron emission tomography (PET imaging) for ¹⁸F, ¹¹C, ⁶⁸Ga, ⁶⁴Cu, ⁸⁹Zr, or ¹²⁴I labeled HER3 targeting agents or single photon emission computed tomography (SPECT imaging) for ^{99m}Tc or ¹¹¹In labeled HER3 targeting agents.

While various specific embodiments have been illustrated and described herein, it will be appreciated that various changes can be made without departing from the spirit and scope of the invention(s). Moreover, features described in connection with one aspect of the invention may be used in conjunction with other aspects of the invention, even if not explicitly exemplified in combination within.

### REFERENCES

Mishra R, Patel H, Alanazi S, Yuan L, Garrett J T. HER3 signaling and targeted therapy in cancer. Oncol Rev. 2018; 12(1).
Meneses-Lorente G, Friess T, Kolm I, et al. Preclinical pharmacokinetics, pharmacodynamics, and efficacy of RG7116: a novel humanized, glycoengineered anti-HER3 antibody. Cancer Chemother Pharmacol. 2015; 75(4):837-850.
Mirschberger C, Schiller C B, Schraml M, et al. RG7116, a Therapeutic Antibody That Binds the Inactive HER3 Receptor and Is Optimized for Immune Effector Activation. Cancer Res. 2013; 73(16):5183-5194.
Meulendijks D, Jacob W, Martinez-Garcia M, et al. First-in-Human Phase I Study of Lumretuzumab, a Glycoengineered Humanized Anti-HER3 Monoclonal Antibody, in Patients with Metastatic or Advanced HER3-Positive Solid Tumors. Clin Cancer Res. 2016; 22(4):877-885.
Reynolds K L, Bedard P L, Lee S-H, et al. A phase I open-label dose-escalation study of the anti-HER3 monoclonal antibody LJM716 in patients with advanced squamous cell carcinoma of the esophagus or head and neck and HER2-overexpressing breast or gastric cancer. BMC Cancer. 2017; 17(1):646.

What is claimed is:

1. A method for treating a solid cancer in a mammalian subject, the method comprising:
administering to the subject a therapeutically effective amount of a radionuclide labeled HER3 targeting agent; and
administering to the subject a therapeutically effective amount of at least one VEGF inhibitor or VEGFR inhibitor or pharmaceutically acceptable salt thereof,
wherein the radionuclide labeled HER3 targeting agent comprises a radiolabeled monoclonal antibody comprising:
(i) one or both of
(a) an immunoglobulin heavy chain variable region including a CDR-H1 including SEQ ID NO:15, a CDR-H2 including SEQ ID NO:16, and/or a CDR-H3 including SEQ ID NO:17, and
(b) an immunoglobulin light chain variable region including a CDR-L1 including SEQ ID NO:18, a CDR-L2 including SEQ ID NO:19, and/or a CDR-L3 including SEQ ID NO:20;
(ii) one or both of an immunoglobulin heavy chain variable region including SEQ ID NO:21 and an immunoglobulin light chain variable region including SEQ ID NO:22;
(iii) one or both of an immunoglobulin heavy chain amino acid sequence of SEQ ID NO:23 and an immunoglobulin light chain amino acid sequence of SEQ ID NO:24;
(iv) one or both of an immunoglobulin heavy chain sequence comprising SEQ ID NO:77 and an immunoglobulin light chain sequence comprising SEQ ID NO:78; or
(v) AV-203, and
wherein the at least one VEGF inhibitor or VEGFR inhibitor comprises one or more of pazopanib; sunitinib; bevacizumab; sorafenib; regorafenib; cabozantinib; lenvatinib; ponatinib; cabozantinib; ziv-aflibercept; axitinib; tivozanib; ramucirumab; vandetanib; erlotinib, dovitinib; vorolanib; ranibizumab; tarcocimab; emvododstat; muparfostat; nintedanib; Sevacizumab; Cediranib; famitinib; zanzalintinib; ibcasertib; lucitanib; sitravatinib; Ilorasertib; Tinengotinib; Tesevatinib; Olinvacimab; Acrizanib; brivanib; telatinib; and altiratinib.

2. The method of claim 1, wherein the radionuclide labeled HER3 targeting agent comprises a radionuclide selected from ⁶⁴Cu, 67Cu, ²⁰¹Tl, ⁴⁷Sc, ⁹⁰Y, ¹⁷⁷Lu, ¹⁸⁶Re, ¹⁸⁸Re, ¹⁵³Sm, ³²P, ²²⁵Ac, ²¹³Bi, ²¹³Po, ²¹¹At, ²¹²Bi, ²¹³Bi, ²²³Ra, ²²⁷Th, ¹⁴⁹Tb, ¹³⁷Cs, ²¹²Pb or ¹⁰³Pd, or any combination thereof.

3. The method of claim 1, wherein the mammalian subject is human.

4. The method of claim 2, wherein the mammalian subject is human.

5. The method of claim 1, wherein the solid cancer is renal cell carcinoma (RCC), metastatic RCC, non-small cell lung cancer (NSCLC), hepatocellular carcinoma (HCC), colorectal carcinoma (CRC), gastric cancer GIST cancer, breast cancer, tamoxifen-resistant breast cancer, metastatic breast cancer, triple-negative breast cancer, ovarian cancer, fallopian tube cancer, primary peritoneal cancer, bladder cancer, prostate cancer, advanced prostate cancer, metastatic prostate cancer, castration resistant prostate cancer, metastatic castration resistant prostate cancer, gall bladder duct cancer, bile duct cancer, osteosarcoma, soft tissue sarcoma, or pancreatic cancer.

6. The method of claim 2, wherein the solid cancer is renal cell carcinoma (RCC), metastatic RCC, non-small cell lung cancer (NSCLC), hepatocellular carcinoma (HCC), colorectal carcinoma (CRC), gastric cancer GIST cancer, breast cancer, tamoxifen-resistant breast cancer, metastatic breast cancer, triple-negative breast cancer, ovarian cancer, fallopian tube cancer, primary peritoneal cancer, bladder cancer, prostate cancer, advanced prostate cancer, metastatic prostate cancer, castration resistant prostate cancer, metastatic castration resistant prostate cancer, gall bladder duct cancer, bile duct cancer, osteosarcoma, soft tissue sarcoma, or pancreatic cancer.

7. The method of claim 3, wherein the solid cancer is renal cell carcinoma (RCC), metastatic RCC, non-small cell lung cancer (NSCLC), hepatocellular carcinoma (HCC), colorectal carcinoma (CRC), gastric cancer GIST cancer, breast cancer, tamoxifen-resistant breast cancer, metastatic breast cancer, triple-negative breast cancer, ovarian cancer, fallopian tube cancer, primary peritoneal cancer, bladder cancer, prostate cancer, advanced prostate cancer, metastatic prostate cancer, castration resistant prostate cancer, metastatic castration resistant prostate cancer, gall bladder duct cancer, bile duct cancer, osteosarcoma, soft tissue sarcoma, or pancreatic cancer.

8. The method of claim 4, wherein the solid cancer is renal cell carcinoma (RCC), metastatic RCC, non-small cell lung cancer (NSCLC), hepatocellular carcinoma (HCC), colorectal carcinoma (CRC), gastric cancer GIST cancer, breast cancer, tamoxifen-resistant breast cancer, metastatic breast cancer, triple-negative breast cancer, ovarian cancer, fallopian tube cancer, primary peritoneal cancer, bladder cancer, prostate cancer, advanced prostate cancer, metastatic prostate cancer, castration resistant prostate cancer, metastatic castration resistant prostate cancer, gall bladder duct cancer, bile duct cancer, osteosarcoma, soft tissue sarcoma, or pancreatic cancer.

9. The method of claim 1, wherein the radionuclide labeled HER3 targeting agent is ²²⁵Ac-labeled, and the effective amount of the ²²⁵Ac-labeled HER3 targeting agent comprises a dose of 0.1 to 50 μCi/kg body weight of the subject, or 0.1 to 5 μCi/kg body weight of the subject, or 5 to 20 μCi/kg subject body weight.

10. The method of claim 1, wherein the radionuclide labeled HER3 targeting agent is ²²⁵Ac-labeled, and the effective amount of the ²²⁵Ac-labeled HER3 targeting agent comprises a dose of 2 μCi to 2 mCi, or 2 μCi to 250 μCi, or 75 μCi to 400 μCi.

11. The method of claim 1, wherein the effective amount of the radionuclide labeled HER3 targeting agent comprises a protein dose of less than 3 mg/kg body weight of the subject, such as from 0.001 mg/kg patient weight to 3.0 mg/kg patient weight, or from 0.005 mg/kg patient weight to 2.0 mg/kg patient weight, or from 0.01 mg/kg patient weight to 1 mg/kg patient weight, or from 0.1 mg/kg patient weight to 0.6 mg/kg patient weight, or 0.3 mg/kg patient weight, or 0.4 mg/kg patient weight, or 0.5 mg/kg patient weight, or 0.6 mg/kg patient weight.

12. The method of claim 1, wherein the radionuclide labeled HER3 targeting agent is administered according to a dosing schedule selected from the group consisting of once every 7, 10, 12, 14, 20, 24, 28, 36, and 42 days throughout a treatment period, wherein the treatment period includes at least two doses.

13. The method of claim 1, further comprising:
administering to the subject a therapeutically effective amount of an immune checkpoint therapy, a DNA damage response inhibitor (DDRi), a CD47 blockade, a chemotherapeutic agent, or any combination thereof.

14. The method of claim 17, wherein
the DDRi comprises a poly(ADP-ribose) polymerase inhibitor (PARPi),
the immune checkpoint therapy comprises an antibody(ies) against PD-1, PD-L1, PD-L2, CTLA-4, or CD137, or any combination of such antibodies, and
the CD47 blockade comprises one or more of magrolimab, lemzoparlimab, AO-176, TTI-621, TTI-622, and a CD47 expression-modulating agent.

15. The method of claim 1 wherein the radionuclide labeled HER3 targeting agent comprises a chemically conjugated chelator group that chelates a radionuclide.

16. The method of claim 1, wherein the administering step comprises:
administering to the subject a therapeutically effective amount of a therapeutic composition comprising a radiolabeled fraction of the HER3 targeting agent and a non-radiolabeled fraction of the HER3 targeting agent.

17. The method of claim 16, wherein the therapeutic composition further comprises at least one pharmaceutically acceptable excipient.

18. The method of claim 1, further comprising the step of: diagnosing the subject with a HER3-positive cancer prior to administering the radiolabeled HER3 targeting agent.

19. The method of claim 3, wherein the step of administering to the subject a therapeutically effective amount of at least one VEGF inhibitor or VEGFR inhibitor comprises administering a therapeutically effective amount of tivozanib or a pharmaceutically acceptable salt thereof to the subject.

20. The method of claim 19, wherein administering a therapeutically effective amount of tivozanib or a pharmaceutically acceptable salt thereof to the subject comprises administering a therapeutically effective amount of 1-{2-chloro-4-[(6,7-dimethoxyquinolin-4-yl)oxy]phenyl}-3-(5-methylisoxazol-3-yl)urea hydrochloride hydrate to the subject.

21. The method of claim 19, wherein the solid cancer is renal cell carcinoma.

22. The method of claim 21, wherein the solid cancer is relapsed and/or refractory renal cell carcinoma.

23. The method of claim 21, wherein the radionuclide labeled HER3 targeting agent comprises a radionuclide selected from ⁶⁴Cu, ⁶⁷Cu, ²⁰¹Tl, ⁴⁷Sc, ⁹⁰Y, ¹⁷⁷Lu, ¹⁸⁶Re, ¹⁸⁸Re, ¹⁵³Sm, ³²p ²²⁵Ac, ²¹³Bi, ²¹³Po, ²¹¹At, ²¹²Bi, ²¹³Bi, ²²³Ra, ²²⁷Th, ¹⁴⁹Tb, ¹³⁷Cs, ²¹²Pb or ¹⁰³Pd or any combination thereof.


SEQUENCE LISTING (118 sequences)

SEQ ID NO: 1 - protein, 5 aa, Mus musculus
SHWLH

SEQ ID NO: 2 - protein, 17 aa, Mus musculus
VLDPSDFYSNYNQNFKG

SEQ ID NO: 3 - protein, 11 aa, Mus musculus
GLLSGDYAMDY

SEQ ID NO: 4 - protein, 16 aa, Mus musculus
RSSQSIVHSNGNTYLE

SEQ ID NO: 5 - protein, 7 aa, Mus musculus
KVSNRFS

SEQ ID NO: 6 - protein, 9 aa, Mus musculus
FQGSYVPWT

SEQ ID NO: 7 - protein, 120 aa, Mus musculus
QVQLQQPGAELVRPGTSVKLSCKASGYTFTSHWLHWVKQRPGQGLEWIGVLDPSDFYSNYNQNFKGKATLTVDTSSSTAYMQLSSLTSEDSAVYYCARGLLSGDYAMDYWGAGTSVTVSS

SEQ ID NO: 8 - protein, 112 aa, Mus musculus
DVLMTQIPLSLPVSLGDQASISCRSSQSIVHSNGNTYLEWYLQKPGQSPKSLIYKVSNRFSGVPDRFSGSGSGTDFTLKISRVEAEDLGVYYCFQGSYVPWTFGGGTKLEIK

SEQ ID NO: 9 - protein, 463 aa, Mus musculus
MGWSCIIVLLVSTATGVHSQVQLQQPGAELVRPGTSVKLSCKASGYTFTSHWLHWVKQRPGQGLEWIGVLDPSDFYSNYNQNFKGKATLTVDTSSSTAYMQLSSLTSEDSAVYYCARGLLSGDYAMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 10 - protein, 238 aa, Mus musculus
MKLPVRLLVLMFWIPASSSDVLMTQIPLSLPVSLGDQASISCRSSQSIVHSNGNTYLEWYLQKPGQSPKSLIYKVSNRFSGVPDRFSGSGSGTDFTLKISRVEAEDLGVYYCFQGSYVPWTFGGGTKLEIKRADAAPTVSIFPPSSEQLTSGGASVVCFLNNFYPRDINVKWKIDGSERQNGVLNSWTDQDSKDSTYSMSSTLTLTKDEYERHNSYTCEATHKTSTSPIVKSFNRNEC

SEQ ID NO: 11 - protein, 444 aa, Mus musculus
QVQLQQPGAELVRPGTSVKLSCKASGYTFTSHWLHWVKQRPGQGLEWIGVLDPSDFYSNYNQNFKGKATLTVDTSSSTAYMQLSSLTSEDSAVYYCARGLLSGDYAMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 12 - protein, 219 aa, Mus musculus
DVLMTQIPLSLPVSLGDQASISCRSSQSIVHSNGNTYLEWYLQKPGQSPKSLIYKVSNRFSGVPDRFSGSGSGTDFTLKISRVEAEDLGVYYCFQGSYVPWTFGGGTKLEIKRADAAPTVSIFPPSSEQLTSGGASVVCFLNNFYPRDINVKWKIDGSERQNGVLNSWTDQDSKDSTYSMSSTLTLTKDEYERHNSYTCEATHKTSTSPIVKSFNRNEC

SEQ ID NO: 13 - protein, 10 aa, Mus musculus
QVQLQQPGAE

SEQ ID NO: 14 - protein, 10 aa, Mus musculus
DVLMTQIPLS

SEQ ID NO: 15 - protein, 5 aa, synthetic construct
  REGION 1..5: note = Description of Artificial Sequence: Synthetic peptide
DYAMS

SEQ ID NO: 16 - protein, 17 aa, synthetic construct
  REGION 1..17: note = Description of Artificial Sequence: Synthetic peptide
TISDGGTYTYYPDSVKG

SEQ ID NO: 17 - protein, 10 aa, synthetic construct
  REGION 1..10: note = Description of Artificial Sequence: Synthetic peptide
EWGDYDGFDY

SEQ ID NO: 18 - protein, 11 aa, synthetic construct
  REGION 1..11: note = Description of Artificial Sequence: Synthetic peptide
RASQEISGYLS

SEQ ID NO: 19 - protein, 7 aa, synthetic construct
  REGION 1..7: note = Description of Artificial Sequence: Synthetic peptide
AASTLDS

SEQ ID NO: 20 - protein, 9 aa, synthetic construct
  REGION 1..9: note = Description of Artificial Sequence: Synthetic peptide
LQYDSYPYT

SEQ ID NO: 21 - protein, 119 aa, synthetic construct
  REGION 1..119: note = Description of Artificial Sequence: Synthetic polypeptide
QVQLVESGGGLVKPGGSLRLSCAASGFTFSDYAMSWIRQAPGKGLEWVSTISDGGTYTYYPDSVKGRFTISRDNAKNSLYLQMNSLRAEDTAVYYCAREWGDYDGFDYWGQGTLVTVSS

SEQ ID NO: 22 - protein, 107 aa, synthetic construct
  REGION 1..107: note = Description of Artificial Sequence: Synthetic polypeptide
DIQMTQSPSSLSASVGDRVTITCRASQEISGYLSWYQQKPGKAPKRLIYAASTLDSGVPSRFSGSGSGTEFTLTISSLQPEDFATYYCLQYDSYPYTFGQGTKLEIK

SEQ ID NO: 23 - protein, 471 aa, synthetic construct
  REGION 1..471: note = Description of Artificial Sequence: Synthetic polypeptide
MDMRVPAQLLGLLLLWLRGARCQVQLVESGGGLVKPGGSLRLSCAASGFTFSDYAMSWIRQAPGKGLEWVSTISDGGTYTYYPDSVKGRFTISRDNAKNSLYLQMNSLRAEDTAVYYCAREWGDYDGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 236 aa, synthetic construct
  REGION 1..236: note = Description of Artificial Sequence: Synthetic polypeptide
MDMRVPAQLLGLLLLWLRGARCDIQMTQSPSSLSASVGDRVTITCRASQEISGYLSWYQQKPGKAPKRLIYAASTLDSGVPSRFSGSGSGTEFTLTISSLQPEDFATYYCLQYDSYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 25 - protein, 5 aa, synthetic construct
  REGION 1..5: note = Description of Artificial Sequence: Synthetic peptide
SHWLH

SEQ ID NO: 26 - protein, 17 aa, synthetic construct
  REGION 1..17: note = Description of Artificial Sequence: Synthetic peptide
VLDPSDFYSNYNQNFKG

SEQ ID NO: 27 - protein, 11 aa, synthetic construct
  REGION 1..11: note = Description of Artificial Sequence: Synthetic peptide
GLLSGDYAMDY

SEQ ID NO: 28 - protein, 16 aa, synthetic construct
  REGION 1..16: note = Description of Artificial Sequence: Synthetic peptide
RSSQSIVHSNGNTYLE

SEQ ID NO: 29 - protein, 7 aa, synthetic construct
  REGION 1..7: note = Description of Artificial Sequence: Synthetic peptide
KVSNRFS

SEQ ID NO: 30 - protein, 9 aa, synthetic construct
  REGION 1..9: note = Description of Artificial Sequence: Synthetic peptide
FQGSYVPWT

SEQ ID NO: 31 - protein, 120 aa, synthetic construct
  REGION 1..120: note = Description of Artificial Sequence: Synthetic polypeptide
QVQLQQPGAELVRPGTSVKLSCKASGYTFTSHWLHWVKQRPGQGLEWIGVLDPSDFYSNYNQNFKGKATLTVDTSSSTAYMQLSSLTSEDSAVYYCARGLLSGDYAMDYWGQGTSVTVSS

SEQ ID NO: 32 - protein, 112 aa, synthetic construct; identical to SEQ ID NO: 8
  REGION 1..112: note = Description of Artificial Sequence: Synthetic polypeptide

SEQ ID NO: 33 - protein, 463 aa, synthetic construct; identical to SEQ ID NO: 9
  REGION 1..463: note = Description of Artificial Sequence: Synthetic polypeptide

SEQ ID NO: 34 - protein, 238 aa, synthetic construct; identical to SEQ ID NO: 10
  REGION 1..238: note = Description of Artificial Sequence: Synthetic polypeptide

SEQ ID NO: 35 - protein, 7 aa, synthetic construct
  REGION 1..7: note = Description of Artificial Sequence: Synthetic peptide
TFGLSVG

SEQ ID NO: 36 - protein, 16 aa, synthetic construct
  REGION 1..16: note = Description of Artificial Sequence: Synthetic peptide
HIWWDDDKYYNPALKS

SEQ ID NO: 37 - protein, 10 aa, synthetic construct
  REGION 1..10: note = Description of Artificial Sequence: Synthetic peptide
IGADALPFDY

SEQ ID NO: 38 - protein, 16 aa, synthetic construct
  REGION 1..16: note = Description of Artificial Sequence: Synthetic peptide
RSSKSLLHSNGNTYLY

SEQ ID NO: 39 - protein, 7 aa, synthetic construct
  REGION 1..7: note = Description of Artificial Sequence: Synthetic peptide
RMSNLAS

SEQ ID NO: 40 - protein, 9 aa, synthetic construct
  REGION 1..9: note = Description of Artificial Sequence: Synthetic peptide
MQHLEYPFT

SEQ ID NO: 41 - protein, 120 aa, synthetic construct
  REGION 1..120: note = Description of Artificial Sequence: Synthetic polypeptide
QVTLKESGPGILRPSQTLSLTCSFSGFSLSTFGLSVGWIRQPSGKGLEWLAHIWWDDDKYYNPALKSRLTISKDTSKNQVFLKIANVDTADTATYYCARIGADALPFDYWGQGTTLTVSS

SEQ ID NO: 42 - protein, 112 aa, synthetic construct
  REGION 1..112: note = Description of Artificial Sequence: Synthetic polypeptide
DIVLTQTAPSVPVTPGESVSISCRSSKSLLHSNGNTYLYWFLQRPGQSPQLLIYRMSNLASGVPDRFSGSGSGTAFTLRISRVEAEDVGVYYCMQHLEYPFTFGSGTKLEIK

SEQ ID NO: 43 - protein, 475 aa, synthetic construct
  REGION 1..475: note = Description of Artificial Sequence: Synthetic polypeptide
MGRLTSSFLLLIVPAYVLSQVTLKESGPGILRPSQTLSLTCSFSGFSLSTFGLSVGWIRQPSGKGLEWLAHIWWDDDKYYNPALKSRLTISKDTSKNQVFLKIANVDTADTATYYCARIGADALPFDYWGQGTTLTVSSAKTTPPSVYPLAPGCGDTTGSSVTSGCLVKGYFPEPVTVTWNSGSLSSSVHTFPALLQSGLYTMSSSVTVPSSTWPSQTVTCSVAHPASSTTVDKKLEPSGPISTINPCPPCKECHKCPAPNLEGGPSVFIFPPNIKDVLMISLTPKVTCVVVDVSEDDPDVQISWFVNNVEVHTAQTQTHREDYNSTIRVVSTLPIQHQDWMSGKEFKCKVNNKDLPSPIERTISKIKGLVRAPQVYTLPPPAEQLSRKDVSLTCLVVGFNPGDISVEWTSNGHTEENYKDTAPVLDSDGSYFIYSKLNMKTSKWEKTDSFSCNVRHEGLKNYYLKKTISRSPGK

SEQ ID NO: 44 - protein, 239 aa, synthetic construct
  REGION 1..239: note = Description of Artificial Sequence: Synthetic polypeptide
MRCLAEFLGLLVLWIPGAIGDIVLTQTAPSVPVTPGESVSISCRSSKSLLHSNGNTYLYWFLQRPGQSPQLLIYRMSNLASGVPDRFSGSGSGTAFTLRISRVEAEDVGVYYCMQHLEYPFTFGSGTKLEIKRADAAPTVSIFPPSSEQLTSGGASVVCFLNNFYPRDINVKWKIDGSERQNGVLNSWTDQDSKDSTYSMSSTLTLTKDEYERHNSYTCEATHKTSTSPIVKSFNRNEC

SEQ ID NO: 45 - protein, 5 aa, synthetic construct
  REGION 1..5: note = Description of Artificial Sequence: Synthetic peptide
DHIIH

SEQ ID NO: 46 - protein, 17 aa, synthetic construct
  REGION 1..17: note = Description of Artificial Sequence: Synthetic peptide
YIYPRDGYIKYNEKFKG

SEQ ID NO: 47 - protein, 8 aa, synthetic construct
  REGION 1..8: note = Description of Artificial Sequence: Synthetic peptide
GYYYAMDY

SEQ ID NO: 48 - protein, 16 aa, synthetic construct
  REGION 1..16: note = Description of Artificial Sequence: Synthetic peptide
RSSQSIVHSIGNTYLE

SEQ ID NO: 49 - protein, 9 aa, synthetic construct
  REGION 1..9: note = Description of Artificial Sequence: Synthetic peptide
FQGSHVPFT

SEQ ID NO: 50 - protein, 117 aa, synthetic construct
  REGION 1..117: note = Description of Artificial Sequence: Synthetic polypeptide
QVQLQQSDAELVKPGASVKISCKVSGYTFTDHIIHWMKQRPEQGLEWIGYIYPRDGYIKYNEKFKGKATLTADKSSSTAYMQVNSLTSEDSAVYFCARGYYYAMDYWGQGTSVTVSS

SEQ ID NO: 51 - protein, 112 aa, synthetic construct
  REGION 1..112: note = Description of Artificial Sequence: Synthetic polypeptide
DVLMTQTPLSLPVSLGDQASISCRSSQSIVHSIGNTYLEWYLQKPGQSPKLLIYKVSNRFSGVPERFSGSGSGTDFTLKISRVEAEDLGVYYCFQGSHVPFTFGSGTKLEIK

SEQ ID NO: 52 - protein, 460 aa, synthetic construct
  REGION 1..460: note = Description of Artificial Sequence: Synthetic polypeptide
MEWSWVSLFFLSVTTGVHSQVQLQQSDAELVKPGASVKISCKVSGYTFTDHIIHWMKQRPEQGLEWIGYIYPRDGYIKYNEKFKGKATLTADKSSSTAYMQVNSLTSEDSAVYFCARGYYYAMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 53 - protein, 238 aa, synthetic construct
  REGION 1..238: note = Description of Artificial Sequence: Synthetic polypeptide
MKLPVRLLVLMFWIPASRSDVLMTQTPLSLPVSLGDQASISCRSSQSIVHSIGNTYLEWYLQKPGQSPKLLIYKVSNRFSGVPERFSGSGSGTDFTLKISRVEAEDLGVYYCFQGSHVPFTFGSGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 54 - protein, 5 aa, synthetic construct
  REGION 1..5: note = Description of Artificial Sequence: Synthetic peptide
SYWMH

SEQ ID NO: 55 - protein, 17 aa, synthetic construct
  REGION 1..17: note = Description of Artificial Sequence: Synthetic peptide
MIDPSDVYTNYNPKFKG

SEQ ID NO: 56 - protein, 6 aa, synthetic construct
  REGION 1..6: note = Description of Artificial Sequence: Synthetic peptide
NYSGDY

SEQ ID NO: 57 - protein, 115 aa, synthetic construct
  REGION 1..115: note = Description of Artificial Sequence: Synthetic polypeptide
QVQLLQPGAELVRPGTSVKLSCKTSGYTFSSYWMHWVKQRPGQGLEWIGMIDPSDVYTNYNPKFKGKATLTVDTSSSTAYMQLSSLTSEDSAVYYCARNYSGDYWGQGTTLTVSS

SEQ ID NO: 58 - protein, 112 aa, synthetic construct
  REGION 1..112: note = Description of Artificial Sequence: Synthetic polypeptide
DVLMTQIPLSLPVSLGDQASISCRSSQSIVHSNGNTYLEWYLQKPGQSPKLLIYKVSNRFSGVPDRFSGSGSGTDFTLKISRVEAEDLGVYYCFQGSYVPWTFGGGTKLEIK

SEQ ID NO: 59 - protein, 458 aa, synthetic construct
  REGION 1..458: note = Description of Artificial Sequence: Synthetic polypeptide
MGWSCIIVLLVSTATCVHSQVQLLQPGAELVRPGTSVKLSCKTSGYTFSSYWMHWVKQRPGQGLEWIGMIDPSDVYTNYNPKFKGKATLTVDTSSSTAYMQLSSLTSEDSAVYYCARNYSGDYWGQGTTLTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 60 - protein, 238 aa, synthetic construct
  REGION 1..238: note = Description of Artificial Sequence: Synthetic polypeptide
MKLPVRLLVLMFWIPASSSDVLMTQIPLSLPVSLGDQASISCRSSQSIVHSNGNTYLEWYLQKPGQSPKLLIYKVSNRFSGVPDRFSGSGSGTDFTLKISRVEAEDLGVYYCFQGSYVPWTFGGGTKLEIKRADAAPTVSIFPPSSEQLTSGGASVVCFLNNFYPRDINVKWKIDGSERQNGVLNSWTDQDSKDSTYSMSSTLTLTKDEYERHNSYTCEATHKTSTSPIVKSFNRNEC

SEQ ID NO: 61 - protein, 5 aa, synthetic construct
  REGION 1..5: note = Description of Artificial Sequence: Synthetic peptide
TYGMS

SEQ ID NO: 62 - protein, 17 aa, synthetic construct
  REGION 1..17: note = Description of Artificial Sequence: Synthetic peptide
WINTYSGVPTYADDFKG

SEQ ID NO: 63 - protein, 12 aa, synthetic construct
  REGION 1..12: note = Description of Artificial Sequence: Synthetic peptide
GRDGYQVAWFAY

SEQ ID NO: 64 - protein, 11 aa, synthetic construct
  REGION 1..11: note = Description of Artificial Sequence: Synthetic peptide
ITSTDIDDDMN

SEQ ID NO: 65 - protein, 7 aa, synthetic construct
  REGION 1..7: note = Description of Artificial Sequence: Synthetic peptide
EGNTLRP

SEQ ID NO: 66 - protein, 9 aa, synthetic construct
  REGION 1..9: note = Description of Artificial Sequence: Synthetic peptide
LQSDNLPYT

SEQ ID NO: 67 - protein, 121 aa, synthetic construct
  REGION 1..121: note = Description of Artificial Sequence: Synthetic polypeptide
QIQLVQSGPELKKPGEAVKISCKSSGYTFTTYGMSWVKQAPGRALKWMGWINTYSGVPTYADDFKGRFAFSLESSASTAYLQINNLKNEDTATYFCARGRDGYQVAWFAYWGQGTLVTVSA

SEQ ID NO: 68 - protein, 107 aa, synthetic construct
  REGION 1..107: note = Description of Artificial Sequence: Synthetic polypeptide
ETTVTQSPASLSMAIGDKVTIRCITSTDIDDDMNWFQQKPGEPPKLLISEGNTLRPGVPSRFSGSGYGTDFIFTIENMLSEDVADYYCLQSDNLPYTFGGGTKLEIK

SEQ ID NO: 69 - protein, 464 aa, synthetic construct
  REGION 1..464: note = Description of Artificial Sequence: Synthetic polypeptide
MGWLWNLLFLMAAAQSAQAQIQLVQSGPELKKPGEAVKISCKSSGYTFTTYGMSWVKQAPGRALKWMGWINTYSGVPTYADDFKGRFAFSLESSASTAYLQINNLKNEDTATYFCARGRDGYQVAWFAYWGQGTLVTVSAAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 70 - protein, 234 aa, synthetic construct
  REGION 1..234: note = Description of Artificial Sequence: Synthetic polypeptide
MFSLALLLSLLLLCVSDSRAETTVTQSPASLSMAIGDKVTIRCITSTDIDDDMNWFQQKPGEPPKLLISEGNTLRPGVPSRFSGSGYGTDFIFTIENMLSEDVADYYCLQSDNLPYTFGGGTKLEIKRADAAPTVSIFPPSSEQLTSGGASVVCFLNNFYPRDINVKWKIDGSERQNGVLNSWTDQDSKDSTYSMSSTLTLTKDEYERHNSYTCEATHKTSTSPIVKSFNRNEC

SEQ ID NO: 71 - protein, 5 aa, synthetic construct
  REGION 1..5: note = Description of Artificial Sequence: Synthetic peptide
NYWMH

SEQ ID NO: 72 - protein, 17 aa, synthetic construct
  REGION 1..17: note = Description of Artificial Sequence: Synthetic peptide
MIDPSDSYTNYNPKFKG

SEQ ID NO: 73 - protein, 115 aa, synthetic construct
  REGION 1..115: note = Description of Artificial Sequence: Synthetic polypeptide
QVQLQQPGAELVRPGTSVKLSCKASGYTFTNYWMHWVKQRPGQGLEWIGMIDPSDSYTNYNPKFKGKATLTVDTSSSTAYMQLSSLTSEDSAVYYCARNYSGDYWGQGTTLTVSS

SEQ ID NO: 74 - protein, 112 aa, synthetic construct
  REGION 1..112: note = Description of Artificial Sequence: Synthetic polypeptide
DVLMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSYVPWTFGGGTKLEIK

SEQ ID NO: 75 - protein, 458 aa, synthetic construct
  REGION 1..458: note = Description of Artificial Sequence: Synthetic polypeptide
MGWSCIIVLLVSTATGVHSQVQLQQPGAELVRPGTSVKLSCKASGYTFTNYWMHWVKQRPGQGLEWIGMIDPSDSYTNYNPKFKGKATLTVDTSSSTAYMQLSSLTSEDSAVYYCARNYSGDYWGQGTTLTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 76 - protein, 238 aa, synthetic construct
  REGION 1..238: note = Description of Artificial Sequence: Synthetic polypeptide
MKLPVRLLVLMFWIPASSSDVLMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSYVPWTFGGGTKLEIKRADAAPTVSIFPPSSEQLTSGGASVVCFLNNFYPRDINVKWKIDGSERQNGVLNSWTDQDSKDSTYSMSSTLTLTKDEYERHNSYTCEATHKTSTSPIVKSFNRNEC

SEQ ID NO: 77 - protein, 449 aa, synthetic construct
  REGION 1..449: note = Antibody heavy chain
QVQLVESGGGLVKPGGSLRLSCAASGFTFSDYAMSWIRQAPGKGLEWVSTISDGGTYTYYPDSVKGRFTISRDNAKNSLYLQMNSLRAEDTAVYYCAREWGDYDGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 78 - protein, 214 aa, synthetic construct
  REGION 1..214: note = Antibody light chain
DIQMTQSPSSLSASVGDRVTITCRASQEISGYLSWYQQKPGKAPKRLIYAASTLDSGVPSRFSGSGSGTEFTLTISSLQPEDFATYYCLQYDSYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 79 - protein, 119 aa, synthetic construct
  REGION 1..119: note = Description of Artificial Sequence: Synthetic polypeptide
EVQLVESGGGLVQPGGSLRLSCAASGFTFSDYAMSWVRQAPGKGLEWVATISDGGTYTYYPDNVKGRFTISRDNAKNSLYLQMNSLRAEDTAVYYCAREWGDYDGFDYWGQGTLVTVSS

SEQ ID NO: 80 - protein, 119 aa, synthetic construct
  REGION 1..119: note = Description of Artificial Sequence: Synthetic polypeptide
QVQLVESGGGLVKPGGSLRLSCAASGFTFSDYAMSWIRQAPGKGLEWVSTISDGGTYTYYPDNVKGRFTISRDNAKNSLYLQMNSLRAEDTAVYYCAREWGDYDGFDYWGQGTLVTVSS

SEQ ID NO: 81 - protein, 119 aa, synthetic construct; identical to SEQ ID NO: 21
  REGION 1..119: note = Description of Artificial Sequence: Synthetic polypeptide

SEQ ID NO: 82 - protein, 119 aa, synthetic construct
  REGION 1..119: note = Description of Artificial Sequence: Synthetic polypeptide
EVQLVESGGGLVKPGGSLRLSCAASGFTFSDYAMSWVRQAPGKGLEWVSTISDGGTYTYYPDNVKGRFTISRDNAKNSLYLQMNSLRAEDTAVYYCAREWGDYDGFDYWGQGTLVTVSS

SEQ ID NO: 83 - protein, 119 aa, synthetic construct
  REGION 1..119: note = Description of Artificial Sequence: Synthetic polypeptide
EVQLLESGGGLVQPGGSLRLSCAASGFTFSDYAMSWVRQAPGKGLEWVSTISDGGTYTYYPDNVKGRFTISRDNSKNTLYLQMNSLRAEDTAVYYCAREWGDYDGFDYWGQGTLVTVSS

SEQ ID NO: 84 - protein, 119 aa, synthetic construct
  REGION 1..119: note = Description of Artificial Sequence: Synthetic polypeptide
QVQLVESGGGVVQPGRSLRLSCAASGFTFSDYAMSWVRQAPGKGLEWVATISDGGTYTYYPDNVKGRFTISRDNSKNTLYLQMSSLRAEDTAVYYCAREWGDYDGFDYWGQGTLVTVSS

SEQ ID NO: 85 - protein, 119 aa, synthetic construct
  REGION 1..119: note = Description of Artificial Sequence: Synthetic polypeptide
EVQLVESGGGLVKPGGSLRLSCAASGFTFSDYAMSWVRQAPGKGLEWVATISDGGTYTYYPDNVKGRFTISRDNAKNSLYLQMNSLRAEDTAVYYCAREWGDYDGFDYWGQGTLVTVSS

SEQ ID NO: 86 - protein, 107 aa, synthetic construct
  REGION 1..107: note = Description of Artificial Sequence: Synthetic polypeptide
DIQLTQSPSFLSASVGDRVTITCRASQEISGYLSWYQQKPGKAPKLLIYAASTLDSGVPSRFSGSGSGTEFTLTISSLQPEDFATYYCLQYDSYPYTFGQGTKLEIK

SEQ ID NO: 87 - protein, 107 aa, synthetic construct
  REGION 1..107: note = Description of Artificial Sequence: Synthetic polypeptide
DIQMTQSPSSLSASVGDRVTITCRASQEISGYLSWFQQKPGKAPKSLIYAASTLDSGVPSRFSGSGSGTDFTLTISSLQPEDFATYYCLQYDSYPYTFGQGTKLEIK

SEQ ID NO: 88 - protein, 107 aa, synthetic construct; identical to SEQ ID NO: 22
  REGION 1..107: note = Description of Artificial Sequence: Synthetic polypeptide

SEQ ID NO: 89 - protein, 107 aa, synthetic construct
  REGION 1..107: note = Description of Artificial Sequence: Synthetic polypeptide
DIQMTQSPSSLSASVGDRVTITCRASQEISGYLSWYQQKPGKAPKLLIYAASTLDSGVPSRFSGSGSGTDFTFTISSLQPEDIATYYCLQYDSYPYTFGQGTKLEIK

SEQ ID NO: 90 - protein, 107 aa, synthetic construct
  REGION 1..107: note = Description of Artificial Sequence: Synthetic polypeptide
DIQMTQSPSSLSASVGDRVTITCRASQEISGYLSWYQQKPGKAPKLLIYAASTLDSGVPSRFSGSGSGTDFTLTISSLQPEDFATYYCLQYDSYPYTFGQGTKLEIK

SEQ ID NO: 91 - protein, 107 aa, synthetic construct
  REGION 1..107: note = Description of Artificial Sequence: Synthetic polypeptide
DIQMTQSPSSLSASVGDRVTITCRASQEISGYLSWLQQKPGGAIKRLIYAASTLDSGVPSRFSGSGSGSDYTLTISSLQPEDFATYYCLQYDSYPYTFGQGTKLEIK

SEQ ID NO: 92 - protein, 468 aa, synthetic construct
  REGION 1..468: note = Description of Artificial Sequence: Synthetic polypeptide
MNFGLSLMFLVLVLKGVQCEVQLVESGGGLVKPGGSLKLSCAASGFTFSDYAMSWVRQTPEKRLEWVATISDGGTYTYYPDNVKGRFTISRDNAKNNLYLQMSHLKSEDTAMYYCAREWGDYDGFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 93 - protein, 236 aa, synthetic construct
  REGION 1..236: note = Description of Artificial Sequence: Synthetic polypeptide
MDMRVPAHVFGFLLLWFPGTRCDIQMTQSPSSLSASLGERVSLTCRASQEISGYLSWLQQKPDGTIKRLIYAASTLDSGVPKRFSGSRSGSDYSLTIGSLESEDLADYYCLQYDSYPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 94 - protein, 471 aa, synthetic construct; identical to SEQ ID NO: 23
  REGION 1..471: note = Description of Artificial Sequence: Synthetic polypeptide

SEQ ID NO: 95 - protein, 467 aa, synthetic construct
  REGION 1..467: note = Description of Artificial Sequence: Synthetic polypeptide
MDMRVPAQLLGLLLLWLRGARCQVQLVESGGGLVKPGGSLRLSCAASGFTFSDYAMSWIRQAPGKGLEWVSTISDGGTYTYYPDSVKGRFTISRDNAKNSLYLQMNSLRAEDTAVYYCAREWGDYDGFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 96 - protein, 236 aa, synthetic construct
  REGION 1..236: note = Description of Artificial Sequence: Synthetic polypeptide
MDMRVPAQLLGLLLLWLRGARCDIQMTQSPSSLSASVGDRVTITCRASQEISGYLSWFQQKPGKAPKSLIYAASTLDSGVPSRFSGSGSGTDFTLTISSLQPEDFATYYCLQYDSYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 97 - protein, 236 aa, synthetic construct; identical to SEQ ID NO: 24
  REGION 1..236: note = Description of Artificial Sequence: Synthetic polypeptide

SEQ ID NO: 98 - protein, 449 aa, synthetic construct
  REGION 1..449: note = Antibody heavy chain no leader sequence
QVQLVESGGGLVKPGGSLRLSCAASGFTFSDYAMSWIRQAPGKGLEWVSTISDGGTYTYYPDSVKGRFTISRDNAKNSLYLQMNSLRAEDTAVYYCAREWGDYDGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 99 - protein, 445 aa, synthetic construct
  REGION 1..445: note = Antibody heavy chain no leader sequence
QVQLVESGGGLVKPGGSLRLSCAASGFTFSDYAMSWIRQAPGKGLEWVSTISDGGTYTYYPDSVKGRFTISRDNAKNSLYLQMNSLRAEDTAVYYCAREWGDYDGFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 100 - protein, 214 aa, synthetic construct
  REGION 1..214: note = Antibody light chain no leader sequence
DIQMTQSPSSLSASVGDRVTITCRASQEISGYLSWFQQKPGKAPKSLIYAASTLDSGVPSRFSGSGSGTDFTLTISSLQPEDFATYYCLQYDSYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 101 - protein, 214 aa, synthetic construct; identical to SEQ ID NO: 78
  REGION 1..214: note = Antibody light chain no leader sequence

SEQ ID NO: 102 - protein, 450 aa, synthetic construct
  REGION 1..450: note = Antibody heavy chain
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 103 - protein, 214 aa, synthetic construct
  REGION 1..214: note = Antibody light chain
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 104 - protein, 448 aa, synthetic construct
  REGION 1..448: note = Antibody heavy chain
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 105 - protein, 214 aa, synthetic construct
  REGION 1..214: note = Antibody light chain
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 106 - protein, 447 aa, synthetic construct
  REGION 1..447: note = Antibody heavy chain
QVQLQQWGAGLLKPSETLSLTCAVYGGSFSGYYWSWIRQPPGKGLEWIGEINHSGSTNYNPSLKSRVTISVETSKNQFSLKLSSVTAADTAVYYCARDKWTWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 107 - protein, 220 aa, synthetic construct
  REGION 1..220: note = Antibody light chain
DIEMTQSPDSLAVSLGERATINCRSSQSVLYSSSNRNYLAWYQQNPGQPPKLLIYWASTRESGVPDRFSGSGSGTDFTLTISSLQAEDVAVYYCQQYYSTPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 108 - protein, 445 aa, synthetic construct
  REGION 1..445: note = Antibody heavy chain
EVQLLESGGGLVQPGGSLRLSCAASGFTFSHYVMAWVRQAPGKGLEWVSSISSSGGWTLYADSVKGRFTISRDNSKNTLYLQMNSLRAEDTAVYYCTRGLKMATIFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 109 - protein, 217 aa, synthetic construct
  REGION 1..217: note = Antibody light chain
QSALTQPASVSGSPGQSITISCTGTSSDVGSYNVVSWYQQHPGKAPKLIIYEVSQRPSGVSNRFSGSKSGNTASLTISGLQTEDEADYYCCSYAGSSIFVIFGGGTKVTVLGQPKAAPSVTLFPPSSEELQANKATLVCLVSDFYPGAVTVAWKADGSPVKVGVETTKPSKQSNNKYAASSYLSLTPEQWKSHRSYSCRVTHEGSTVEKTVAPAECS

SEQ ID NO: 110 - protein, 449 aa, synthetic construct
  REGION 1..449: note = Antibody heavy chain
QVQLVQSGAEVKKPGASVKVSCKASGYTFRSSYISWVRQAPGQGLEWMGWIYAGTGSPSYNQKLQGRVTMTTDTSTSTAYMELRSLRSDDTAVYYCARHRDYYSNSLTYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 111 - protein, 220 aa, synthetic construct
  REGION 1..220: note = Antibody light chain
DIVMTQSPDSLAVSLGERATINCKSSQSVLNSGNQKNYLTWYQQKPGQPPKLLIYWASTRESGVPDRFSGSGSGTDFTLTISSLQAEDVAVYYCQSDYSYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 112 - protein, 446 aa, synthetic construct
  REGION 1..446: note = Antibody heavy chain
EVQLLESGGGLVQPGGSLRLSCAASGFTFSSYAMSWVRQAPGKGLEWVSAINSQGKSTYYADSVKGRFTISRDNSKNTLYLQMNSLRAEDTAVYYCARWGDEGFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 113 - protein, 214 aa, synthetic construct
  REGION 1..214: note = Antibody light chain
DIQMTQSPSSLSASVGDRVTITCRASQGISNWLAWYQQKPGKAPKLLIYGASSLQSGVPSRFSGSGSGTDFTLTISSLQPEDFATYYCQQYSSFPTTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 114 - DNA, 25 bp, synthetic construct
  misc_feature 1..25: note = Morpholino
  source 1..25: mol_type = other DNA
cgtcacaggcaggacccactgccca

SEQ ID NO: 115 - protein, 1342 aa, Homo sapiens
MRANDALQVLGLLFSLARGSEVGNSQAVCPGTLNGLSVTGDAENQYQTLYKLYERCEVVMGNLEIVLTGHNADLSFLQWIREVTGYVLVAMNEFSTLPLPNLRVVRGTQVYDGKFAIFVMLNYNTNSSHALRQLRLTQLTEILSGGVYIEKNDKLCHMDTIDWRDIVRDRDAEIVVKDNGRSCPPCHEVCKGRCWGPGSEDCQTLTKTICAPQCNGHCFGPNPNQCCHDECAGGCSGPQDTDCFACRHFNDSGACVPRCPQPLVYNKLTFQLEPNPHTKYQYGGVCVASCPHNFVVDQTSCVRACPPDKMEVDKNGLKMCEPCGGLCPKACEGTGSGSRFQTVDSSNIDGFVNCTKILGNLDFLITGLNGDPWHKIPALDPEKLNVFRTVREITGYLNIQSWPPHMHNFSVFSNLTTIGGRSLYNRGFSLLIMKNLNVTSLGFRSLKEISAGRIYISANRQLCYHHSLNWTKVLRGPTEERLDIKHNRPRRDCVAEGKVCDPLCSSGGCWGPGPGQCLSCRNYSRGGVCVTHCNFLNGEPREFAHEAECFSCHPECQPMEGTATCNGSGSDTCAQCAHFRDGPHCVSSCPHGVLGAKGPIYKYPDVQNECRPCHENCTQGCKGPELQDCLGQTLVLIGKTHLTMALTVIAGLVVIFMMLGGTFLYWRGRRIQNKRAMRRYLERGESIEPLDPSEKANKVLARIFKETELRKLKVLGSGVFGTVHKGVWIPEGESIKIPVCIKVIEDKSGRQSFQAVTDHMLAIGSLDHAHIVRLLGLCPGSSLQLVTQYLPLGSLLDHVRQHRGALGPQLLLNWGVQIAKGMYYLEEHGMVHRNLAARNVLLKSPSQVQVADFGVADLLPPDDKQLLYSEAKTPIKWMALESIHFGKYTHQSDVWSYGVTVWELMTFGAEPYAGLRLAEVPDLLEKGERLAQPQICTIDVYMVMVKCWMIDENIRPTFKELANEFTRMARDPPRYLVIKRESGPGIAPGPEPHGLTNKKLEEVELEPELDLDLDLEAEEDNLATTTLGSALSLPVGTLNRPRGSQSLLSPSSGYMPMNQGNLGESCQESAVSGSSERCPRPVSLHPMPRGCLASESSEGHVTGSEAELQEKVSMCRSRSRSRSPRPRGDSAYHSQRHSLLTPVTPLSPPGLEEEDVNGYVMPDTHLKGTPSSREGTLSSVGLSSVLGTEEEDEDEEYEYMNRRRRHSPPHPPRPSSLEELGYEYMDVGSDLSASLGSTQSCPLHPVPIMPTAGTTPDEDYEYMNRQRDGGGPGGDYAAMGACPASEQGYEEMRAFQGPGHQAPHVHYARLKTLRSLEATDSAFDNPDYWHSRLFPKANAQRT

SEQ ID NO: 116 - protein, 345 aa, synthetic construct
  REGION 1..345: note = Fusion protein
EEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKPSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 117 - protein, 345 aa, synthetic construct; identical to SEQ ID NO: 116
  REGION 1..345: note = Fusion protein

SEQ ID NO: 118 - protein, 347 aa, synthetic construct
  REGION 1..347: note = Fusion protein
EEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKPSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK